(19) United States
(12) Patent Application Publication
(10) Pub. No.: US 2026/0232640 A1
(43) Pub. Date: Aug. 13, 2026

(54) COMPOSITIONS AND METHODS FOR TREATING SPINAL MUSCULAR ATROPHY

(71) Applicant: NMD Pharma A/S, Aarhus N (DK)

(72) Inventors: Jorge Armando Quiroz BASTIAS, Aarhus N (DK); Thomas Holm PEDERSEN, Aarhus N (DK); Thomas Skjærlund GRØNNEBÆK, Aarhus N (DK); Eva Rose CHIN, Palm Desert, CA (US); Jane Mary BOLD, Buckinghamshire (GB); Martin Brandhøj SKOV, Aarhus N (DK)

(73) Assignee: NMD Pharma A/S, Aarhus N (DK)

(21) Appl. No.: 19/160,063

(22) PCT Filed: Mar. 1, 2024
(86) PCT No.: PCT/EP2024/055308

§ 371 Date: Aug. 27, 2025

Related U.S. Application Data
(60) Provisional application No. 63/488,055, filed on Mar. 2, 2023.

(30) Foreign Application Priority Data
Mar. 16, 2023 (EP) 23162356.2
Nov. 8, 2023 (EP) 23208554.8

(51) Int. Cl. A61K 31/42; A61K 31/501; A61K 31/519; A61K 31/712; A61K 31/7125; A61P 21/00; A61P 25/28

(52) U.S. Cl. CPC A61K 31/42; A61K 31/501; A61K 31/519; A61K 31/712; A61K 31/7125; A61P 21/00; A61P 25/28

(57) ABSTRACT

Disclosed herein are methods of treatment for spinal muscular atrophy (SMA) that involve administering (2S)-2-[4-bromo-2-(1,2-oxazol-3-yl)phenoxy]propanoic acid, or a pharmaceutical composition thereof, to a patient suffering from symptoms of SMA. Pharmaceutical compositions and kits-to-parts including (2S)-2-[4-bromo-2-(1,2-oxazol-3-yl)phenoxy]propanoic acid are also disclosed.

### TECHNICAL FIELD

The present disclosure relates to (2S)-2-[4-bromo-2-(1,2-oxazol-3-yl)phenoxy]propanoic acid for use in the treatment of spinal muscular atrophy (SMA), its pharmaceutical composition for use in the treatment of SMA and methods of treatment thereof.

### BACKGROUND

Spinal muscular atrophy (SMA) is a rare neuromuscular disorder that results in the loss of motor neurons and progressive muscle wasting. It is usually diagnosed in infancy or early childhood and if left untreated it is the most common genetic cause of infant death. It may also appear later in life and then has a milder course of disease. The common feature is progressive weakness of voluntary muscles, with arm, leg and respiratory muscles being affected first. Associated problems may include poor head control, difficulties swallowing, scoliosis, and joint contractures.

Spinal muscular atrophy is due to a mutation in the SMN1 gene which encodes SMN, a protein necessary for survival of motor neurons. Loss of motor neurons results in failure to transmit electrical signals that must propagate from the brain to skeletal muscle fibres in order to activate their contractions. SMN2 is another gene that has evolved in human. While it is a duplicate gene it is not identical to SMN1. SMN2 gene has a single nucleotide difference in exon 7 meaning that the protein product is different from SMN1 and SMN2. Thus, around 90% of the SMN protein product from SMN2 is truncated and non-functional, while the remaining SMN protein product from SMN2 is intact and fully functional. Given that different people have different number of gene copies of SMN2, it is considered a disease modifying gene in SMA. Individuals with more SMN2 copies have more intact SMN protein from SMN2, and the more SMN2 gene copies a SMA patient has the better protected the patient will be from the loss of SMN1 function, and the milder the disease can be expected to be.

There are 5 types of SMA; type 0, type 1 (also called Werdnig-Hoffmann disease), type 2 (also called Dubowitz disease), type 3 (also called Kugelberg-Welander disease) and type 4. SMA Type 0 is usually prenatal, and if untreated children usually survive only a few weeks even with 24/7 respiratory support. SMA Type 1 is diagnosed in ca. 50% of patients and age of onset is usually 0-6 months. Untreated babies diagnosed with SMA Type 1 do not generally survive past two years of age. SMA Type 2 is diagnosed in ca. 20% of patients and age of onset is usually 6-18 months. Patients can typically maintain a sitting position but will never learn to walk unaided. Diseases progression in patients with SMA Type 2 varies but typically patients live well into adulthood although body muscles are weakened. The respiratory system is a major concern as are muscle contractures and spinal curvature. SMA Type 3 is diagnosed in ca. 30% of patients and age of onset is usually >12 months. The disease progresses slowly, and most people with SMA Type 3 lose walking ability sometime in their lives, requiring mobility support. SMA Type 4 is diagnosed in ca. 5% of patients and usually manifests in the third or fourth decade of life. The symptoms consist of gradual weakening of leg muscles, which frequently makes it necessary for the patient to use walking aids.

Recently approved therapies include: 1) an antisense oligonucleotide (ASO) to enhance correct splicing of SMN2 (nusinersen), 2) gene therapy to provide an alternative source of SMN1 gene to produce intact SMN protein (onasemnogene abeparvovec-xioi) used in patients up to the age of 2 years old, and 3) a small molecule to enhance SMN2 splicing (risdiplam).

Patients with SMA show fatigability when tested in a continuous, repetitive task (Stam et al 2018a), a symptom that has been linked to failing neuromuscular junction (NMJ) function (Bartels et al 2019). Although current standard-of-care approaches that target upregulation of SMN have shown promise (Maggi et al 2020), recent neurophysiological data suggest that many adult SMA patients continue to exhibit evidence of NMJ deficits despite up to 14 months of ASO therapy (Arnold et al 2021). Furthermore, patients receiving ASO therapy showed persistent deficits in clinical outcome measures, including the 6-minute walk test, that correlated with measures of NMJ function such as compound muscle action potential (CMAP) decrements (Arnold et al 2021). The presence of such neuromuscular junction deficits in SMA patients and their persistence even after receiving SMN upregulating therapy suggest potential benefits of administering therapies targeting NMJ activity as both a mono therapy and as an add-on therapy to SMN upregulating therapy.

Skeletal muscle specific CIC-1 chloride ion channels carry inhibitory currents at the NMJ that counteract neuromuscular transmission. Inhibition of CIC-1 reduces the inhibitory current and thereby increases muscle membrane excitability and enhances neuromuscular transmission. This was shown to lead to recovery of muscle function under conditions mimicking neuromuscular disorders (Pedersen et al 2021).

(2S)-2-[4-Bromo-2-(1,2-oxazol-3-yl)phenoxy]propanoic acid, hereinafter NMD670, is an inhibitor of the skeletal muscle-specific CIC-1 channel. The chemical structure of NMD670 is provided below.

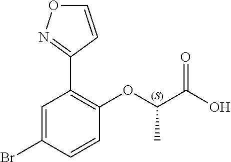

### (2S)-2-[4-bromo-2-(1,2-oxazol-3-yl)phenoxy]propanoic acid (NMD670)

NMD670 alters the voltage sensitivity of the CIC-1 channel resulting in decreased membrane conductance for Cl⁻ in muscle fibres and increased muscle fibre excitability. CIC-1 inhibition with NMD670 restores muscle activation under conditions of failing neuromuscular transmission or compromised muscle fibre excitability and under these conditions, NMD670 can restore force production of skeletal muscle.

Accordingly, the CIC-1 channel is emerging as a target for potential drugs, although its potential has been largely unrealized. U.S. Pat. No. 10,385,028 discloses the synthesis of compounds which have been designed to inhibit the action of the CIC-1 channel to treat neuromuscular disorders. One of the compounds discussed in U.S. Pat. No. 10,385,028 is NMD670.

Though U.S. Pat. No. 10,385,028 discloses a set of compounds which can inhibit the CIC-1 channel to treat neuromuscular disorders, there is no discussion into how to design treatment methods to where these compounds can effectively alleviate the wide range of symptoms associated with SMA. WO2020/254554, herein incorporated by reference, discloses methods for manufacturing NMD670.

Accordingly, there is a need for safe and efficacious therapies to improve muscle function in patients with all forms of SMA, including those patients who have undergone a SMN1 gene therapy and/or are treated with SMN2 upregulating therapies.

### SUMMARY

The present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein the composition is for administration at a therapeutic dose of 100 to 1500 mg of (2S)-2-[4-bromo-2-(1,2-oxazol-3-yl)phenoxy]propanoic acid. The present disclosure further relates to a kit-of-parts comprising (2S)-2-[4-bromo-2-(1,2-oxazol-3-yl)phenoxy]propanoic acid, or a pharmaceutically acceptable salt, hydrate, polymorph, tautomer, or solvate thereof, and a compound that increases the amount of functional SMN protein produced by SMN2.

### DESCRIPTION OF DRAWINGS

FIG. 1

FIG. 1 depicts recordings of Compound Muscle Action Potentials (CMAP) from 67-day old SMA (delta-7 mouse) animals. The mice had been treated with morpholino ASOs directed against ISS-N1 to increase full-length SMN protein production from SMN2 on post-natal day 2 to ensure survival to around 90 days. CMAP recordings were obtained from triceps surae muscle and elicited by electrical stimulation of the sciatic nerve at 50 Hz for 10 stimuli. Evidence of NMJ defects before treatment (left (grey) trace, pre-treatment) can be seen from the successive decline in amplitude of CMAP with repeated stimulation of the suppling nerve (see methods). After treatment with CIC-1 inhibitor the amplitude is similar on all 10 stimulations with only minimal decrement visible (right (black) trace) showing enhanced neuromuscular transmission.

FIG. 2

FIG. 2 depicts the average levels of decrement in CMAP amplitude from 1^{st} to 10^{th} nerve-stimulation when elicited at a range of frequencies from 10 to 50 Hz (as in FIG. 1). Pre-treatment (grey squares) and post treatment with CIC-1 inhibitor (black triangles) are shown. The relative amplitude from the 10th to the 1st stimulation is denoted as decrement, and a larger negative number designates a larger deficit/decrement, indicating a larger neuromuscular transmission failure from nerve to muscle. The decrement was less pronounced in all animals at all frequencies but was only statistically significantly different between pre-treated and treated animals from 20 to 50 Hz, and not at 10 Hz.

FIG. 3

FIG. 3 depicts the latency to fall in seconds from 9-week-old SMA (delta-7 mouse) animals, treated with morpholino ASOs directed against ISS-N1 to increase full-length SMN protein production from SMN2 on post-natal day 2, before (grey bar) and after treatment with CIC-1 inhibitor (black bar). All animals were able to stay on the rotating rod longer after treatment.

FIG. 4

FIG. 4 depicts the study design of a double-blinded, placebo-controlled, two-way cross-over study of male and female ambulatory participants diagnosed with Type 3 SMA with neuromuscular junction (NMJ) deficits. Abbreviations: BL=baseline; EOS=end-of-study; EOT=end-of-treatment; R=Randomisation; V=Visit.

FIGS. 5A and 5B

FIGS. 5 and 5B depict the schedule of activities during the clinical trial. Abbreviations: 6MWT=6-minute walk test; AE=adverse event; bid=twice daily dosing; CMAP=compound muscle action potential; C-SSRS=Columbia-Suicide Severity Rating Scale; D=Day; ECG=electrocardiogram; EOS=end-of-study; EOT=end of treatment; ESNHPT=Endurance Shuttle Nine Hole Peg Test: FSS=fatigue severity scale; HIV=human immunodeficiency virus; INQoL=Individualised Neuromuscular Quality of Life; MFM-32=Motor Function Measure 32 item; PK=pharmacokinetic(s); qd=once daily dosing; RNS=repeated nerve stimulation; SAE=serious adverse event; sfEMG=single fibre electromyography; SMA=spinal muscular atrophy; V=Visit; WOCBP=woman of childbearing potential.

### NOTES

If possible, assessments will be performed in the order presented in the table, unless specified otherwise. If possible, 6MWT will be performed 2 hours after the afternoon dosing.
Visit 6 will occur after a washout period of 6 (±1) days after Visit 5, the day before the start of Treatment Period 2, so the inter-treatment period between the last day of Treatment Period 1 and the first day of Treatment Period 2 is 7 (±1) days.
During each treatment period, the participants will be contacted by phone at 7 and 14 days of treatment.
If possible, RNS assessments will be conducted after confirming other eligibility criteria are fulfilled to avoid unnecessary testing.

### Definitions

All publications, patent applications, patents and other references mentioned herein are incorporated by reference in their entirety.

The nomenclature used in the present application is based on IUPAC systematic nomenclature, unless indicated otherwise.

The term “patient” or “subject” refers to a human (such as a male or female human) who has been diagnosed with spinal muscular atrophy (SMA). A consensus document on the diagnosis of children with SMA was initially developed (Wang et al 2007) and was later updated (Mercuri et al 2018). Confirmation of diagnosis can be performed by genetic testing (i.e., homozygous deletion of survival of motor neuron 1 gene [SMN1]).

The term “improvement” refers to a lessening of a patient's spinal muscular atrophy (SMA) symptoms when the patient is administered a composition as described herein. The improvement may be lessening of a patient's SMA symptoms after the patient has been administered a composition as described herein as compared to before the administration of said composition. The term “improvement” may also refer to lessening of a group of patients' SMA symptoms after the group of patients has been administered a composition as described herein, e.g. as evaluated based on comparative test scores between the patient group being administered the composition as described herein with a control group receiving e.g. placebo. An improvement in SMA symptoms can be determined for example as an increase in the total distance walked during a 6-minute walk test; an increase in muscle strength determined by measuring grip strength, elbow flexor/extensor strength, knee flexor strength, and/or shoulder abduction strength using a handheld dynamometer; an increase in the revised Hammersmith scale score; an improvement in endurance in the endurance shuttle nine hole peg test; a reduction in drop-out rate in the endurance shuttle nine hole peg test; a reduction in fatigue when determined using a fatigue index calculated from the 6-minute walk test; an increase in the motor function measure 32-item score; a decrease in the Fatigue Severity Scale score; a decrease in the Individualised Neuromuscular Quality of Life score; a reduction in jitter; a reduction in blocking; an increase in the Children's Hospital of Philadelphia Infant Test of Neuromuscular Disorders score; an increase in the Hammersmith Functional Motor Scale score; an increase in the expanded Hammersmith Functional Motor Scale score; an increase in the Hammersmith Infant Neurological Examination score; an increase in the PedsQL Neuromuscular Model score; an increase in the Patient Reported Outcomes Measurement Information System score; an increase in the Revised Upper Limb Module score; and/or an increase in the WHO Multicentre Growth Reference Study score. In an exemplary embodiment, an improvement in SMA symptoms includes an increase in the total distance walked during a 6-minute walk test.

The term “jitter” refers to the variability in the arrival time of muscle fibre action potentials to the recording electrode between consecutive electrical discharges when measuring neuromuscular function using single fiber electromyography (sfEMG).

The term “blocking” refers to complete NMJ transmission failure of muscle fibre action potentials to the recording electrode between consecutive electrical discharges when measuring neuromuscular function using sfEMG.

The term “placebo” refers to a dosage form possessing no therapeutic activity.

The term “active pharmaceutical ingredient” (or “API”) denotes the compound or molecule in a pharmaceutical composition that has a particular biological activity.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “pharmaceutical composition” refers to a preparation which is in such form as to permit the biological activity of an active ingredient contained therein to be effective, and which contains no additional components which are unacceptably toxic to a subject to which the composition would be administered.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

A “pharmaceutically acceptable carrier” refers to an ingredient in a pharmaceutical composition, other than an active ingredient, which is nontoxic to a subject. A pharmaceutically acceptable carrier includes, but is not limited to, a buffer or acidifier, excipient, stabilizer, or preservative.

The term “solid dosage form releases” means the amount of compound that is released or dissolved into solution after a specified period of time when using a United States Pharmacopeia (USP) type 2 dissolution apparatus, paddle speed of 75 rpm, at a temperature of 37° C.±0.5° C. in 900 mL of pH 6.8 phosphate/citric acid buffer as described in Example 10.

The term “Cₘₐₓ” (expressed in units of ng/mL) means maximum observed plasma concentration of NMD670. The term “mean Cₘₐₓ” means the arithmetic mean of the individual Cₘₐₓ values.

The term “Tₘₐₓ” (expressed in units of hours, or as a median number of hours for Tₘₐₓ in the study population) means the observed time to reach Cₘₐₓ following drug administration; if it occurs at more than one time point Tₘₐₓ is defined as the first time point with this value.

The term “dose” means the dose of NMD670 as free acid that was given to the subject. In addition, the term “dose’ may be inclusive of NMD670 in combination with a pharmaceutically acceptable salt.

The term “therapeutically effective dose” as used herein refers to the amount of NMD670 required to cause a therapeutic response in a subject. The terms “therapeutically effective dose” and “therapeutic dose” are used interchangeably herein.

The composition comprising the (therapeutic) dose may be administered in one or more unit dosage forms. As used herein, “unit dosage forms” refers to physically discrete units suitable for human and animal subjects. Each unit dosage includes a predetermined quantity of the therapeutically active compound, in association with, when required, a pharmaceutical carrier, vehicle or diluent. Examples of unit dosage forms include tablets, capsules, pills, powders, granules, sterile parenteral solutions or suspensions, ampoules and syringes, and oral solutions or suspensions, and oil-water emulsions. Unit dosage forms can be individually packaged as is known in the art, such as in blister packs. Unit dosage forms can be administered in fractions or multiples thereof.

The term “T_{1/2}” (expressed in units of hours) means the terminal elimination half-life of NMD670 in plasma.

The term “AUC_{0-infinity}” (expressed in units of h·ng/mL) means the cumulative area under the plasma time concentration curve (AUC) calculated using the trapezoidal method from time 0 to infinity after a single dose of NMD670. The term “mean AUC_{0-infinity}” means the arithmetic mean of the individual AUC_{0-infinity} values.

The term “AUC₀₋₂₄ hours” (expressed in units of h·ng/mL) means the cumulative area under the plasma time concentration curve (AUC) calculated using the trapezoidal method from time 0 to 24 hours after a single dose of NMD670. The term “mean AUC₀₋₂₄ₕₒᵤᵣₛ” means the arithmetic mean of the individual AUC₀₋₂₄ₕₒᵤᵣₛ values.

As used within the following disclosure, the term “NMD670” refers to (2S)-2-[4-bromo-2-(1,2-oxazol-3-yl)phenoxy]propanoic acid, in addition to any pharmaceutically acceptable salt, hydrate, polymorph, tautomer, or solvate thereof.

(2S)-2-[4-bromo-2-(1,2-oxazol-3-yl)phenoxy]propanoic acid, according to the present disclosure, refers to a compound of formula (I) below, CAS Number 2354321-33-6.

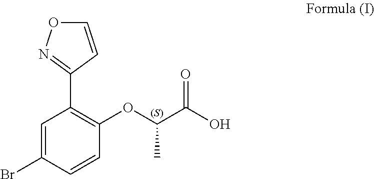

All publications, patent applications, patents and other references mentioned herein are incorporated by reference in their entirety.

### DETAILED DESCRIPTION

Prior preclinical and clinical studies have shown that NMJ transmission defects are evident in animal models of SMA (Kong et al, 2009; Foust et al, 2010) and in patients affected by SMA (Wadman et al, 2012; Pera et al, 2017; Arnold et al, 2021). In particular, it has been shown that the 6-minute walk test (6MWT) can identify fatigue in ambulant Type 3 patients who have a concurrent neuromuscular junction dysfunction (Pera et al, 2017) and that this disfunction is still apparent even after patients have been treated with nusinersen, a SMN upregulating therapy (Arnold et al, 2021).

Herein, we demonstrate that after administration of a CIC-1 inhibitor in a mouse model of SMA (Foust et al, 2010), decrement was surprisingly improved (75% improvement at 50 Hz), and this improvement in decrement was associated with improvement in running distance.

The inventors were able to develop the compositions for use in SMA treatment methods and SMA treatment methods described herein. Exemplified embodiments of these compositions and methods are provided below. The compositions and methods described herein are not intended to be limited to the following exemplary embodiments.

### Compositions for Use

One aspect of the present disclosure relates to compositions for use in a method of treatment of spinal muscular atrophy in a subject. Said method comprises administering a therapeutically effective dose of (2S)-2-[4-bromo-2-(1,2-oxazol-3-yl)phenoxy]propanoic acid (NMD670) to the patient, wherein the therapeutically effective dose for the treatment of spinal muscular atrophy is within the range of 100 mg to 1500 mg.

Thus, one aspect of the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein the composition is for administration at a therapeutic dose of 100 to 1500 mg of (2S)-2-[4-bromo-2-(1,2-oxazol-3-yl)phenoxy]propanoic acid.

In exemplary embodiments, the therapeutically effective dose of NMD670 that is administered to the patient is less than 1500 mg, less than 1450 mg, less than 1300 mg, less than 1250 mg, less than 1200 mg, less than 1150 mg, less than 1100 mg, less than 1050 mg, less than 1000 mg, less than 950 mg, less than 900 mg, less than 850 mg, less than 800 mg, less than 750 mg, less than 700 mg, less than 650 mg, less than 600 mg, less than 550 mg, less than 500 mg, less than 450 mg, less than 400 mg, less than 350 mg, less than 300 mg, or less than 250 mg.

In exemplary embodiments, the therapeutically effective dose of NMD670 that is administered to the patient is at least 100 mg, at least 150 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1000 mg, at least 1050 mg, at least 1100 mg, at least 1150 mg, at least 1200 mg, at least 1250 mg, at least 1300 mg, at least 1350 mg, at least 1400 mg, or at least 1450 mg.

In exemplary embodiments, the therapeutically effective dose of NMD670 that is administered to the patient is from 100 to 600 mg, from 200 to 600 mg, from 250 to 550 mg, from 300 to 500 mg, from 350 to 450 mg, from 375 to 425 mg, such as about 400 mg. In other exemplary embodiments, the therapeutically effective dose of NMD670 that is administered to the patient is from 700 to 1400 mg, from 800 to 1350 mg, from 900 to 1300 mg, from 1000 to 1250 mg, from 1100 to 1250 mg, such as about 1200 mg. In exemplary embodiments, the therapeutically effective dose of NMD670 that is administered to the patient is about 100 mg. In exemplary embodiments, the therapeutically effective dose of NMD670 that is administered to the patient is about 150 mg. In exemplary embodiments, the therapeutically effective dose of NMD670 that is administered to the patient is about 200 mg. In exemplary embodiments, the therapeutically effective dose of NMD670 that is administered to the patient is about 250 mg. In exemplary embodiments, the therapeutically effective dose of NMD670 that is administered to the patient is about 300 mg. In exemplary embodiments, the therapeutically effective dose of NMD670 that is administered to the patient is about 350 mg. In exemplary embodiments, the therapeutically effective dose of NMD670 that is administered to the patient is about 400 mg. In exemplary embodiments, the therapeutically effective dose of NMD670 that is administered to the patient is about 500 mg. In exemplary embodiments, the therapeutically effective dose of NMD670 that is administered to the patient is about 600 mg.

In exemplary embodiments, the therapeutic dose is to be administered at least one time daily. In exemplary embodiments, the therapeutic dose is to be administered one time daily. In exemplary embodiments, the therapeutic dose is to be administered two times daily. In exemplary embodiments, the therapeutic dose is to be administered three times daily. In exemplary embodiments, the therapeutic dose is to be administered four times daily.

In exemplary embodiments, the therapeutic dose is administered one time daily, i.e. the therapeutic dose is the total daily dosage. In exemplary embodiments, the therapeutic dose is 100 to 600 mg and is administered one time daily. In exemplary embodiments, the therapeutic dose is 200 to 600 mg and is administered one time daily. In exemplary embodiments, the therapeutic dose is 300 to 500 mg and is administered one time daily. In exemplary embodiments, the therapeutic dose is about 100 mg and is administered one time daily. In exemplary embodiments, the therapeutic dose is about 150 mg and is administered one time daily. In exemplary embodiments, the therapeutic dose is about 200 mg and is administered one time daily. In exemplary embodiments, the therapeutic dose is about 250 mg and is administered one time daily. In exemplary embodiments, the therapeutic dose is about 300 mg and is administered one time daily. In exemplary embodiments, the therapeutic dose is about 350 mg and is administered one time daily. In exemplary embodiments, the therapeutic dose is about 400 mg and is administered one time daily. In exemplary embodiments, the therapeutic dose is about 500 mg and is administered one time daily. In exemplary embodiments, the therapeutic dose is about 600 mg and is administered one time daily.

In exemplary embodiments, the therapeutic dose is administered two times daily, i.e. the total daily dosage is twice the therapeutic dose. In exemplary embodiments, the therapeutic dose is 100 to 600 mg and is administered two times daily. In exemplary embodiments, the therapeutic dose is 200 to 600 mg and is administered two times daily. In exemplary embodiments, the therapeutic dose is 300 to 500 mg and is administered two times daily. In exemplary embodiments, the therapeutic dose is about 100 mg and is administered two times daily. In exemplary embodiments, the therapeutic dose is about 150 mg and is administered two times daily. In exemplary embodiments, the therapeutic dose is about 200 mg and is administered two times daily. In exemplary embodiments, the therapeutic dose is about 250 mg and is administered two times daily. In exemplary embodiments, the therapeutic dose is about 300 mg and is administered two times daily. In exemplary embodiments, the therapeutic dose is about 350 mg and is administered two times daily. In exemplary embodiments, the therapeutic dose is about 400 mg and is administered two times daily. In exemplary embodiments, the therapeutic dose is about 500 mg and is administered two times daily. In exemplary embodiments, the therapeutic dose is about 600 mg and is administered two times daily.

In exemplary embodiments, the therapeutic dose is administered three times daily, i.e. the total daily dosage is three times the therapeutic dose. In exemplary embodiments, the therapeutic dose is 100 to 600 mg and is administered three times daily. In exemplary embodiments, the therapeutic dose is 200 to 600 mg and is administered three times daily. In exemplary embodiments, the therapeutic dose is 300 to 500 mg and is administered three times daily. In exemplary embodiments, the therapeutic dose is about 100 mg and is administered three times daily. In exemplary embodiments, the therapeutic dose is about 150 mg and is administered three times daily. In exemplary embodiments, the therapeutic dose is about 200 mg and is administered three times daily. In exemplary embodiments, the therapeutic dose is about 250 mg and is administered three times daily. In exemplary embodiments, the therapeutic dose is about 300 mg and is administered three times daily. In exemplary embodiments, the therapeutic dose is about 350 mg and is administered three times daily. In exemplary embodiments, the therapeutic dose is about 400 mg and is administered three times daily. In exemplary embodiments, the therapeutic dose is about 500 mg and is administered three times daily. In exemplary embodiments, the therapeutic dose is about 600 mg and is administered three times daily.

The composition comprising the therapeutic dose may be administered in one or more unit dosage forms. A therapeutic dose of 400 mg may for example be administered as one unit dosage form comprising 400 mg, or two unit dosage forms comprising 200 mg, or four unit dosage forms comprising 100 mg.

In exemplary embodiments, the therapeutic dose is the total daily dosage.

In exemplary embodiments, the composition for use is administered orally. In exemplary embodiments, the composition for use is a solid dosage form. In exemplary embodiments, the solid dosage form is administered orally. In exemplary embodiments, the solid dosage form is selected from the group consisting of capsule (such as sprinkle capsule and gelatine capsule), tablet (such as uncoated tablet, coated tablet, slow-release tablet) and sprinkle. In exemplary embodiments, the composition is in the form of a liquid, liquid suspension, oil, emulsion, or syrup. In exemplary embodiments, the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.

In exemplary embodiments, the composition for use is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 12,760 to 27,440 ng/mL, such as 15,000 to 25,000 ng/mL, such as 16,000 to 24,000 ng/mL, such as 16,080 to 25,125 ng/mL, such as 17,000 to 23,000 ng/mL, such as 18,000 to 22,000 ng/mL, such as 19,000 to 21,000 ng/mL, such as about 20,100 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, mean Cₘₐₓ is 20,100 ng/mL and the standard deviation is 7,340 ng/mL.

In exemplary embodiments, the composition for use is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is about 80% to about 125%, such as 80.00% to 125.00%, of 20,100 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is 66,300 to 109,100 h·ng/mL, such as 70,000 to 105,000 h·ng/mL, such as 70,160 to 109,625 h·ng/mL, such as 75,000 to 100,000 h·ng/mL, such as 80,000 to 95,000 h·ng/mL, such as 85,000 to 90,000 h·ng/mL, such as about 87,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, mean AUC_{0-infinity} is 87,700 ng/mL and the standard deviation is 21,400 ng/mL.

In exemplary embodiments, the composition for use is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is about 80% to about 125%, such as 80.00% to 125.00%, of 87,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition or use is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 1 to 6 hours after administration, such as after about 2 hours after administration.

In exemplary embodiments, the composition or use is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 3 to 7 hours after administration.

In exemplary embodiments, the AUC₀₋₂₄, AUC_{0-infinity}, Cₘₐₓ or Tₘₐₓ is measured after administration of a single dose to a human subject suffering from spinal muscular atrophy.

The compositions for use described herein can be formulated for administrating either orally, parenterally, intravenously, through inhalation, topically, enterally, rectally, buccally or as an aerosol.

In exemplary embodiments, the composition for use further comprises at least one pharmaceutically acceptable adjuvant and/or excipient. In exemplary embodiments, the composition for use comprises at least one pharmaceutically acceptable adjuvant and/or excipient selected from the group consisting of filler, binder, lubricant and disintegrant. In exemplary embodiments, the composition for use comprises at least one pharmaceutically acceptable adjuvant and/or excipient selected from the group consisting of silicified microcrystalline cellulose, microcrystalline cellulose, maltodextrin, magnesium stearate and croscarmellose sodium.

In exemplary embodiments, the composition for use comprises 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use comprises 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use is in the form of one or more solid dosage forms comprising:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant; and
e. 0.25 to 5 wt %, such as 0.3 to 2.5 wt % disintegrant;
with the proviso that the sum of the wt % of the components does not exceed 100 wt %.

In exemplary embodiments, the composition for use is in the form of one or more solid dosage forms comprising:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant;
e. 0.25 to 5 wt %, such as 0.3 to 2.5 wt % disintegrant; and
f. 1 to 10 wt % film coating;
with the proviso that the sum of the wt % of the components does not exceed 100 wt %.

In exemplary embodiments, the composition for use is in the form of one or more solid dosage forms comprising:
a. 10 to 80 wt %, such as 40 to 65 wt %, such as 50 to 60 wt %, such as 50 to 55 wt %, such as 55 to 60 wt % such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
with the proviso that the sum of the wt % of the components does not exceed 100 wt %.

In exemplary embodiments, the composition for use is in the form of a solid dosage form and comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
with the proviso that the sum of the wt % of the components does not exceed 100 wt %.

In exemplary embodiments, the composition for use is in the form of one or more solid dosage forms comprising or consisting of:
a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
g. 1 to 10 wt % film coating composition such as Opadry white,
with the proviso that the sum of the wt % of the components does not exceed 100 wt %.

In exemplary embodiments, the composition for use is in the form of a solid dosage form and comprises or consists of:
a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate;
f. 0.25 to 5 wt %, such as 0.3 to 2.5 wt % Croscarmellose sodium; and
g. 1 to 10 wt % film coating composition such as Opadry white,
with the proviso that the sum of the wt % of the components does not exceed 100 wt %.

In exemplary embodiments, the subject has a level of serum uric acid below 6.5 mg/dL.

In exemplary embodiments, the subject has been diagnosed with SMA. In exemplary embodiments, the subject has a deletion or mutation in each survival motor neuron 1 (SMN1) allele. In exemplary embodiments, the subject is homozygous for a SMN1 gene mutation.

In exemplary embodiments, the subject has been diagnosed with SMA type 0. In exemplary embodiments, the subject has been diagnosed with SMA type 0 and has undergone a gene therapy (such as onasemnogene abeparvovec-xioi) to provide an alternative source the SMN1 gene leading to increased SMN protein production. In exemplary embodiments, the subject has been diagnosed with SMA type 0, has undergone a gene therapy (such as onasemnogene abeparvovec-xioi) to provide an alternative source the SMN1 gene leading to increased SMN protein production and is treated with a therapy that increases the amount of functional SMN protein produced by SMN2 (such as nusinersen and/or risdiplam). In exemplary embodiments, the subject has been diagnosed with SMA type 0 and is treated with a therapy that increases the amount of functional SMN protein produced by SMN2 (such as nusinersen and/or risdiplam).

In exemplary embodiments, the subject has been diagnosed with SMA type 1. In exemplary embodiments, the subject has been diagnosed with SMA type 1 and has undergone a gene therapy (such as onasemnogene abeparvovec-xioi) to provide an alternative source the SMN1 gene leading to increased SMN protein. In exemplary embodiments, the subject has been diagnosed with SMA type 1, has undergone a gene therapy (such as onasemnogene abeparvovec-xioi) to provide an alternative source the SMN1 gene leading to increased SMN protein production and is treated with a therapy that increases the amount of functional SMN protein produced by SMN2 (such as nusinersen and/or risdiplam). In exemplary embodiments, the subject has been diagnosed with SMA type 1 and is treated with a therapy that increases the amount of functional SMN protein produced by SMN2 (such as nusinersen and/or risdiplam).

In exemplary embodiments, the subject has been diagnosed with SMA type 2. In exemplary embodiments, the subject has been diagnosed with SMA type 2 and has undergone a gene therapy (such as onasemnogene abeparvovec-xioi) to provide an alternative source the SMN1 gene leading to increased SMN protein production. In exemplary embodiments, the subject has been diagnosed with SMA type 2, has undergone a gene therapy (such as onasemnogene abeparvovec-xioi) to provide an alternative source the SMN1 gene leading to increased SMN protein production and is treated with a therapy that increases the amount of functional SMN protein produced by SMN2 (such as nusinersen and/or risdiplam). In exemplary embodiments, the subject has been diagnosed with SMA type 2 and is treated with a therapy that increases the amount of functional SMN protein produced by SMN2 (such as nusinersen and/or risdiplam).

In exemplary embodiments, the subject has been diagnosed with SMA type 3. In exemplary embodiments, the subject has been diagnosed with SMA type 3 and has undergone a gene therapy (such as onasemnogene abeparvovec-xioi) to provide an alternative source the SMN1 gene leading to increased SMN protein production. In exemplary embodiments, the subject has been diagnosed with SMA type 3, has undergone a gene therapy (such as onasemnogene abeparvovec-xioi) to provide an alternative source the SMN1 gene leading to increased SMN protein production and is treated with a therapy that increases the amount of functional SMN protein produced by SMN2 (such as nusinersen and/or risdiplam). In exemplary embodiments, the subject has been diagnosed with SMA type 3 and is treated with a therapy that increases the amount of functional SMN protein produced by SMN2 (such as nusinersen and/or risdiplam).

In exemplary embodiments, the subject has been diagnosed with SMA Type 4. In exemplary embodiments, the subject has been diagnosed with SMA Type 4 and is treated with a therapy that increases the amount of functional SMN protein produced by SMN2 (such as nusinersen and/or risdiplam).

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered one time daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered one time daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered two times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered two times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered three times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered three times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered four times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered four times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered one time daily; and
the composition is in the form of a solid dosage form and is to be administered orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily; and
the composition is in the form of a solid dosage form and is to be administered orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered three times daily; and
the composition is in the form of a solid dosage form and is to be administered orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered four times daily; and
the composition is in the form of a solid dosage form and is to be administered orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered one time daily; and
the composition is in the form of a solid dosage form and is to be administered orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily; and
the composition is in the form of a solid dosage form and is to be administered orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered three times daily; and
the composition is in the form of a solid dosage form and is to be administered orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered four times daily; and
the composition is in the form of a solid dosage form and is to be administered orally.

In exemplary embodiments, the subject experiences a lessening of spinal muscular atrophy symptoms.

In exemplary embodiments, the subject or group of subjects experience an increase in the total distance walked after treatment with NMD670. An increase in the total distance walked can be determined using a 6-minute walk test (ATS, 2002). In exemplary embodiments, an increase in the total distance walked can be determined by comparing the change from baseline in the total distance walked after a defined period of time (e.g., 21 days) of NMD670 treatment with the change from baseline in the total distance walked after a defined period of time (e.g., 21 days) of placebo treatment. In exemplary embodiments, the subject or group of subjects experience an increase in the total distance walked when determined using the 6-minute walk test after treatment with NMD670. In exemplary embodiments, the subject or group of subjects experience an increase in the total distance walked when determined using the 6-minute walk test after treatment with NMD670, wherein the total distance walked has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 400%, such as between 5% and 200%, such as between 10% and 200%. In exemplary embodiments, the subject or group of subjects experience an increase in the total distance walked when determined using the 6-minute walk test after treatment with NMD670, wherein the total distance walked has increased by at least 20 metres, such as at least 30 metres, such as at least 40 metres, such as at least 50 metres, such as at least 60 metres, such as at least 80 metres, such as at least 100 metres, such as at least 150 metres, such as at least 200 metres, such as at least 250 metres, such as at least 300 metres, such as between 20 and 400 metres, such as between 30 and 300 metres, such as between 40 and 200 metres.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the total distance walked when determined using the 6-minute walk test.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the total distance walked when determined using the 6-minute walk test.

In exemplary embodiments, the subject or group of subjects experience an increase in muscle strength after treatment with NMD670. Grip strength is one measure of muscular strength and can be used to determine the maximum force/tension generated by one's forearm muscles. Grip strength can be used as a screening tool for the measurement of upper body strength and overall strength. In exemplary embodiments, increase in muscle strength can also be determined by measuring the strength of the thigh (knee flexors), the upper arm (elbow flexor and extension) and/or the shoulder (shoulder abduction). In exemplary embodiments, the subject or group of subjects experience an increase in muscle strength after treatment with NMD670 when determined by measuring grip strength using a handheld dynamometer (Febrer et al, 2010; Merlini et al, 2002). In exemplary embodiments, an increase in muscle strength can be determined by comparing the change from baseline in muscle strength after a defined period of time (e.g., 21 days) of NMD670 treatment with the change from baseline in muscle strength after a defined period of time (e.g., 21 days) of placebo treatment. In exemplary embodiments the subject or group of subjects experience an increase in muscle strength by at least 5%, such as at least 10%, such as at least 15%, such as at least 20%, such as at least 25%, such as at least 30%, such as at least 50%, such as at least 75%, such as at least 100%, such as at least 150%, such as at least 200% such as between 10% and 400%, such as between 15% and 200%, such as between 20% and 100%. In exemplary embodiments the subject or group of subjects experience an increase in muscle strength after treatment with NMD670 when determined by measuring muscle strength using a handheld dynamometer, wherein muscle strength has increased by at least 5%, such as at least 10%, such as at least 15%, such as at least 20%, such as at least 25%, such as at least 30%, such as at least 50%, such as at least 75%, such as at least 100%, such as at least 150%, such as at least 200% such as between 10% and 400%, such as between 15% and 200%, such as between 20% and 100%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in muscle strength.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in muscle strength.

In exemplary embodiments the subject or group of subjects experience an increase in muscle strength after treatment with NMD670 when determined by measuring hand grip strength using a handheld dynamometer, wherein hand grip strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments the subject or group of subjects experience an increase in muscle strength after treatment with NMD670 when determined by measuring knee flexor strength using a handheld dynamometer, wherein knee flexor strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as at least 5.0 kg, such as at least 7.5 kg, such as between 0.25 and 15.0 kg, such as between 0.25 and 10.0 kg, such as between 0.5 and 5.0 kg.

In exemplary embodiments the subject or group of subjects experience an increase in muscle strength after treatment with NMD670 when determined by measuring elbow flexor strength using a handheld dynamometer, wherein elbow flexor strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in muscle strength when determined by measuring muscle strength using a handheld dynamometer.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in muscle strength when determined by measuring muscle strength using a handheld dynamometer.

In exemplary embodiments, the subject or group of subjects experience an increase in muscle strength after treatment with NMD670. In exemplary embodiments, the subject or group of subjects experience an increase in muscle strength after treatment with NMD670 when determined by measuring muscle strength using a fixed dynamometer (e.g. an isokinetic dynamometer) (Anders et al, 2012; Harbo et al, 2012). In exemplary embodiments, an increase in muscle strength can be determined by comparing the change from baseline in muscle strength after a defined period of time (e.g., 21 days) of NMD670 treatment with the change from baseline in muscle strength after a defined period of time (e.g., 21 days) of placebo treatment. In exemplary embodiments the subject or group of subjects experience an increase in muscle strength after treatment with NMD670 when determined by measuring muscle strength using a fixed dynamometer, wherein muscle strength has increased by at least 5%, such as at least 10%, such as at least 15%, such as at least 20%, such as at least 25%, such as at least 30%, such as at least 50%, such as at least 75%, such as at least 100%, such as at least 150%, such as at least 200% such as between 10% and 400%, such as between 15% and 200%, such as between 20% and 100%.

In exemplary embodiments the subject or group of subjects experience an increase in muscle strength after treatment with NMD670 when determined by measuring muscle strength of the ankle dorsiflexion using a fixed dynamometer, wherein ankle dorsiflexion strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in isometric strength when determined by measuring muscle strength using a fixed dynamometer.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in isometric strength when determined by measuring muscle strength using a fixed dynamometer.

In exemplary embodiments, the subject or group of subjects experience an increase in the revised Hammersmith scale score after treatment with NMD670 (Ramsey et al 2017). In exemplary embodiments, an increase in the revised Hammersmith scale score can be determined by comparing the change from baseline in the revised Hammersmith scale score after a defined period of time (e.g., 21 days) of NMD670 treatment with the change from baseline in the revised Hammersmith scale score after a defined period of time (e.g., 21 days) of placebo treatment. In exemplary embodiments the subject or group of subjects experience an increase in the revised Hammersmith scale score after treatment with NMD670, wherein the score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the revised Hammersmith scale score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the revised Hammersmith scale score.

In exemplary embodiments, the subject or group of subjects experience an improvement in endurance after treatment with NMD670. In exemplary embodiments, the subject experiences an improvement in endurance after treatment with NMD670 when determined using an endurance shuttle nine hole peg test (Bartels et al, 2019; Bartels et al, 2020). In exemplary embodiments, an improvement in endurance can be determined by comparing the change from baseline in the endurance shuttle nine hole peg test after a defined period of time (e.g., 21 days) of NMD670 treatment with the change from baseline in the endurance shuttle nine hole peg test after a defined period of time (e.g., 21 days) of placebo treatment. In exemplary embodiments, the subject or group of subjects experience an improvement in endurance after treatment with NMD670 when determined using an endurance shuttle nine hole peg test, wherein endurance has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 400%, such as between 10% and 300%, such as between 10% and 200%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an improvement in endurance when determined using an endurance shuttle nine hole peg test.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an improvement in endurance when determined using an endurance shuttle nine hole peg test.

In exemplary embodiments, the subject or group of subjects experience a reduction in drop-out rate in the endurance shuttle nine hole peg test after treatment with NMD670 (Bartels et al, 2020). In exemplary embodiments, a reduction in drop-out rate can be determined by comparing the change from baseline in the drop-out rate in the endurance shuttle nine hole peg test after a defined period of time (e.g., 21 days) of NMD670 treatment with the change from baseline in the drop-out rate in the endurance shuttle nine hole peg test after a defined period of time (e.g., 21 days) of placebo treatment. In exemplary embodiments, the subject or group of subjects experience a reduction in drop-out rate in the endurance shuttle nine hole peg test after treatment with NMD670, wherein drop-out has decreased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 100%, such as between 10% and 80%, such as between 5% and 60%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a reduction in drop-out rate in the endurance shuttle nine hole peg test.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a reduction in drop-out rate in the endurance shuttle nine hole peg test.

In exemplary embodiments, the subject or group of subjects experience a reduction in fatigue after treatment with NMD670 when determined using a fatigue index. The fatigue index is calculated from the distance walked in the 1st minute to the distance walked in the 6th minute of a 6-minute walk test. In exemplary embodiments, a reduction in fatigue can be determined by comparing the change from baseline in the fatigue index after a defined period of time (e.g., 21 days) of NMD670 treatment with the change from baseline in the fatigue index after a defined period of time (e.g., 21 days) of placebo treatment. In exemplary embodiments the subject or group of subjects experience a reduction in fatigue after treatment with NMD670 when determined using a fatigue index, wherein fatigue has decreased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a reduction in fatigue when determined using a fatigue index.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a reduction in fatigue when determined using a fatigue index.

In exemplary embodiments, the subject or group of subjects experience an increase in the motor function measure 32-item score after treatment with NMD670 (Vuillerot et al 2013). In exemplary embodiments, an increase in the motor function measure 32-item score can be determined by comparing the change from baseline in the motor function measure 32-item score after a defined period of time (e.g., 21 days) of NMD670 treatment with the change from baseline in the motor function measure 32-item score after a defined period of time (e.g., 21 days) of placebo treatment. In exemplary embodiments, the subject or group of subjects experience an increase in the motor function measure 32-item score after treatment with NMD670, wherein the score has increased by at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the motor function measure 32-item score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the motor function measure 32-item score.

In exemplary embodiments, the subject or group of subjects experience a decrease in the Fatigue Severity Scale score after treatment with NMD670 (Kizina et al 2020). In exemplary embodiments, a decrease in the Fatigue Severity Scale score can be determined by comparing the change from baseline in the Fatigue Severity Scale score after a defined period of time (e.g., 21 days) of NMD670 treatment with the change from baseline the Fatigue Severity Scale score after a defined period of time (e.g., 21 days) of placebo treatment. In exemplary embodiments, the subject or group of subjects experience a decrease in the Fatigue Severity Scale score after treatment with NMD670, wherein the score has decreased by 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a decrease in the Fatigue Severity Scale score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a decrease in the Fatigue Severity Scale score.

In exemplary embodiments, the subject or group of subjects experience a decrease in the Individualised Neuromuscular Quality of Life score after treatment with NMD670 (Vincent et al 2007). In exemplary embodiments, a decrease in the Individualised Neuromuscular Quality of Life score can be determined by comparing the change from baseline in the Individualised Neuromuscular Quality of Life score after a defined period of time (e.g., 21 days) of NMD670 treatment with the change from baseline the Individualised Neuromuscular Quality of Life score after a defined period of time (e.g., 21 days) of placebo treatment. In exemplary embodiments, the subject or group of subjects experience a decrease in the Individualised Neuromuscular Quality of Life score after treatment with NMD670, wherein the score has decreased by 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a decrease in the Individualised Neuromuscular Quality of Life score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a decrease in the Individualised Neuromuscular Quality of Life score.

In exemplary embodiments, the subject or group of subjects experience a reduction in jitter after treatment with NMD670. In exemplary embodiments, the subject or group of subjects experience a reduction in jitter after treatment with NMD670 when determined using single fibre electromyography (Sanders et al, 2019). In exemplary embodiments, a reduction in jitter can be determined by comparing the change from baseline in jitter after a defined period of time (e.g., 21 days) of NMD670 treatment with the change from baseline in jitter after a defined period of time (e.g., 21 days) of placebo treatment. In exemplary embodiments, the subject or group of subjects experience a reduction in jitter after treatment with NMD670 when determined using single fibre electromyography, wherein jitter has been reduced by at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments, the subject or group of subjects experience a reduction in jitter after treatment with NMD670 when determined using single fibre electromyography, wherein jitter has been reduced by at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs, such as between 5 μs and 200 μs, such as between 5 μs and 100 μs, such as between 10 μs and 50 μs.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a reduction in jitter when determined using single fibre electromyography.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a reduction in jitter when determined using single fibre electromyography.

In exemplary embodiments, the subject or group of subjects experience a reduction in blocking after treatment with NMD670. In exemplary embodiments, the subject or group of subjects experience a reduction in blocking after treatment with NMD670 when determined using single fibre electromyography (Sanders et al, 2019). In exemplary embodiments, a reduction in blocking can be determined by comparing the change from baseline in blocking after a defined time (e.g., 21 days) of NMD670 treatment with the change from baseline in blocking after the same defined time (e.g., 21 days) of placebo treatment. In exemplary embodiments, the subject or group of subjects experience a reduction in blocking after treatment with NMD670 when determined using single fibre electromyography, wherein blocking has been reduced by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a reduction in blocking when determined using single fibre electromyography.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a reduction in blocking when determined using single fibre electromyography.

In exemplary embodiments, the subject or group of subjects experience an increase in the Children's Hospital of Philadelphia Infant Test of Neuromuscular Disorders (CHOP INTEND) score after treatment with NMD670 (Glanzman et al, 2010). In exemplary embodiments, an increase in the CHOP INTEND score can be determined by comparing the change from baseline in the CHOP INTEND score after a defined period of time (e.g., 21 days) of NMD670 treatment with the change from baseline in the CHOP INTEND score after a defined period of time (e.g., 21 days) of placebo treatment. In exemplary embodiments the subject or group of subjects experience an increase in the CHOP INTEND score after treatment with NMD670, wherein the score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the Children's Hospital of Philadelphia Infant Test of Neuromuscular Disorders score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the Children's Hospital of Philadelphia Infant Test of Neuromuscular Disorders score.

In exemplary embodiments, the subject or group of subjects experience an increase in the Hammersmith Functional Motor Scale (HFMS) score after treatment with NMD670 (Main et al, 2003). In exemplary embodiments, an increase in the HFMS score can be determined by comparing the change from baseline in the HFMS score after a defined period of time (e.g., 21 days) of NMD670 treatment with the change from baseline in the HFMS score after a defined period of time (e.g., 21 days) of placebo treatment. In exemplary embodiments the subject or group of subjects experience an increase in the HFMS score after treatment with NMD670, wherein the score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the Hammersmith Functional Motor Scale score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the Hammersmith Functional Motor Scale score.

In exemplary embodiments, the subject or group of subjects experience an increase in the expanded Hammersmith Functional Motor Scale (HFMSE) score after treatment with NMD670 (O'Hagen et al, 2007). In exemplary embodiments, an increase in the HFMSE score can be determined by comparing the change from baseline in the HFMSE score after a defined period of time (e.g., 21 days) of NMD670 treatment with the change from baseline in the HFMSE score after a defined period of time (e.g., 21 days) of placebo treatment. In exemplary embodiments the subject or group of subjects experience an increase in the HFMSE score after treatment with NMD670, wherein the score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the expanded Hammersmith Functional Motor Scale score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the expanded Hammersmith Functional Motor Scale score.

In exemplary embodiments, the subject or group of subjects experience an increase in the Hammersmith Infant Neurological Examination (HINE) score after treatment with NMD670 (De Sanctis et al, 2016). In exemplary embodiments, an increase in the HINE score can be determined by comparing the change from baseline in the HINE score after a defined period of time (e.g., 21 days) of NMD670 treatment with the change from baseline in the HINE score after a defined period of time (e.g., 21 days) of placebo treatment. In exemplary embodiments the subject or group of subjects experience an increase in the HINE score after treatment with NMD670, wherein the score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the Hammersmith Infant Neurological Examination score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the Hammersmith Infant Neurological Examination score.

In exemplary embodiments, the subject or group of subjects experience an increase in the PedsQL Neuromuscular Model score after treatment with NMD670 (Mapi Research Trust, Lyon, France). In exemplary embodiments, an increase in the PedsQL Neuromuscular Model score can be determined by comparing the change from baseline in the PedsQL Neuromuscular Model score after a defined period of time (e.g., 21 days) of NMD670 treatment with the change from baseline in the PedsQL Neuromuscular Model score after a defined period of time (e.g., 21 days) of placebo treatment. In exemplary embodiments the subject or group of subjects experience an increase in the PedsQL Neuromuscular Model score after treatment with NMD670, wherein the score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the PedsQL Neuromuscular Model score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the PedsQL Neuromuscular Model score.

In exemplary embodiments, the subject or group of subjects experience an increase in the Patient Reported Outcomes Measurement Information System (PROMIS) score after treatment with NMD670 (Rodday et al, 2017). In exemplary embodiments, an increase in the PROMIS score can be determined by comparing the change from baseline in the PROMIS score after a defined period of time (e.g., 21 days) of NMD670 treatment with the change from baseline in the PROMIS score after a defined period of time (e.g., 21 days) of placebo treatment. In exemplary embodiments the subject or group of subjects experience an increase in the PROMIS score after treatment with NMD670, wherein the score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the Patient Reported Outcomes Measurement Information System score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the Patient Reported Outcomes Measurement Information System score.

In exemplary embodiments, the subject or group of subjects experience an increase in the Revised Upper Limb Module (RULM) score after treatment with NMD670 (Mazzone et al, 2016). In exemplary embodiments, an increase in the RULM score can be determined by comparing the change from baseline in the RULM score after a defined period of time (e.g., 21 days) of NMD670 treatment with the change from baseline in the RULM score after a defined period of time (e.g., 21 days) of placebo treatment. In exemplary embodiments the subject or group of subjects experience an increase in the RULM score after treatment with NMD670, wherein the score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the Revised Upper Limb Module score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the Revised Upper Limb Module score.

In exemplary embodiments, the subject or group of subjects experience an increase in the WHO Multicentre Growth Reference Study (MGRS) score after treatment with NMD670 (WHO, 2006). In exemplary embodiments, an increase in the MGRS score can be determined by comparing the change from baseline in the MGRS score after a defined period of time (e.g., 21 days) of NMD670 treatment with the change from baseline in the MGRS score after a defined period of time (e.g., 21 days) of placebo treatment. In exemplary embodiments the subject or group of subjects experience an increase in the MGRS score after treatment with NMD670, wherein the score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the WHO Multicentre Growth Reference Study score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an increase in the WHO Multicentre Growth Reference Study score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein the subject has a level of serum uric acid below 6.5 mg/dL. In exemplary embodiments, the composition for use is for administration at a therapeutic dose as defined herein. In exemplary embodiments, the composition for use is for administration at a therapeutic dose of 100 to 1500 mg of (2S)-2-[4-bromo-2-(1,2-oxazol-3-yl)phenoxy]propanoic acid.

In one aspect, the present disclosure relates to use of a composition comprising (2S)-2-[4-bromo-2-(1,2-oxazol-3-yl)phenoxy]propanoic acid, or a pharmaceutically acceptable salt, hydrate, polymorph, tautomer, or solvate thereof, in the manufacture of a medicament for treatment of spinal muscular atrophy in a subject, wherein the composition is for administration at a therapeutic dose of 100 to 1500 mg of (2S)-2-[4-bromo-2-(1,2-oxazol-3-yl)phenoxy]propanoic acid.

In one aspect, the present disclosure relates to (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein (2S)-2-[4-bromo-2-(1,2-oxazol-3-yl)phenoxy]propanoic acid is for administration at a therapeutic dose of 100 to 1500 mg.

### Pharmaceutical Compositions

Another aspect of the present disclosure relates to compositions comprising a therapeutically effective dose of NMD670 for use in treating or ameliorating symptoms of spinal muscular atrophy in a patient suffering from spinal muscular atrophy. A further aspect of the present disclosure relates to compositions comprising a therapeutically effective dose of NMD670 in treating or ameliorating symptoms of spinal muscular atrophy in a patient suffering from spinal muscular atrophy. All of the following exemplified embodiments of the composition can be used in the treatment methods described herein.

In one aspect, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof. In one embodiment, the composition comprises 50 to 400 mg (2S)-2-[4-bromo-2-(1,2-oxazol-3-yl)phenoxy]propanoic acid, or a pharmaceutically acceptable salt, hydrate, polymorph, tautomer, or solvate thereof. In one aspect, the present disclosure relates to a composition, formulated as a solid dosage form, comprising (2S)-2-[4-bromo-2-(1,2-oxazol-3-yl)phenoxy]propanoic acid, or a pharmaceutically acceptable salt, hydrate, polymorph, tautomer, or solvate thereof, wherein the composition comprises 50 to 400 mg (2S)-2-[4-bromo-2-(1,2-oxazol-3-yl)phenoxy]propanoic acid. In exemplary embodiments, the composition further comprises at least one pharmaceutically acceptable adjuvant and/or excipient. In exemplary embodiments, the composition is for oral administration. In exemplary embodiments, the composition further comprises a pharmaceutically acceptable adjuvant and/or excipient selected from the group consisting of filler, binder, lubricant and disintegrant. In exemplary embodiments, the composition comprises a pharmaceutically acceptable adjuvant and/or excipient selected from the group consisting of silicified microcrystalline cellulose, microcrystalline cellulose, maltodextrin, magnesium stearate and croscarmellose sodium. In one embodiment, (2S)-2-[4-bromo-2-(1,2-oxazol-3-yl)phenoxy]propanoic acid, or a pharmaceutically acceptable salt, hydrate, polymorph, tautomer, or solvate thereof, is the only active pharmaceutical ingredient of the composition.

In exemplary embodiments, the composition comprises 50 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 100 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 150 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 200 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 250 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 300 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 350 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 400 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition comprises 10% to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 10% to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant; and
e. 0.25 to 5 wt %, such as 0.3 to 2.5 wt % disintegrant;
with the proviso that the sum of the wt % of the components does not exceed 100 wt %.

In exemplary embodiments, the composition comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant;
e. 0.25 to 5 wt %, such as 0.3 to 2.5 wt % disintegrant; and
f. 1 to 10 wt % film coating;
with the proviso that the sum of the wt % of the components does not exceed 100 wt %.

In exemplary embodiments, the composition comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
with the proviso that the sum of the wt % of the components does not exceed 100 wt %.

In exemplary embodiments, the composition comprises or consists of:
a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin; and
e. 0.25 to 3 wt %, such as 0.4 to 2.0 wt % magnesium stearate;
f. 0.25 to 5 wt %, such as 0.3 to 2.5 wt % Croscarmellose sodium; and
g. 1 to 10 wt % film coating composition such as Opadry white,
with the proviso that the sum of the wt % of the components does not exceed 100 wt %.

In exemplary embodiments, the present disclosure relates to a composition comprising a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg.

In exemplary embodiments, the present disclosure relates to a composition, formulated as a solid dosage form, comprising a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 50 mg to 400 mg. In exemplary embodiments, the therapeutically effective dose is 100 mgs. In exemplary embodiments, the therapeutically effective dose is 150 mgs. In exemplary embodiments, the therapeutically effective dose is 200 mgs. In exemplary embodiments, the therapeutically effective dose is 250 mgs. In exemplary embodiments, the therapeutically effective dose is 300 mgs. In exemplary embodiments, the therapeutically effective dose is 350 mgs. In exemplary embodiments, the therapeutically effective dose is 400 mgs. In exemplary embodiments, the therapeutically effective dose is given once daily. In exemplary embodiments, the therapeutically effective dose is given twice daily. In exemplary embodiments, the therapeutically effective dose is given three times daily. In exemplary embodiments, the therapeutically effective dose is given four times daily.

In exemplary embodiments, the present disclosure relates to a composition, formulated as a solid dosage form, comprising 50 mg to 400 mg of (2S)-2-[4-bromo-2-(1,2-oxazol-3-yl)phenoxy]propanoic acid or a pharmaceutically acceptable salt, hydrate, polymorph, tautomer, or solvate thereof. In exemplary embodiments, the solid dosage form comprises 100 mg of NMD670. In exemplary embodiments, the solid dosage form comprises 150 mg of NMD670. In exemplary embodiments, the solid dosage form comprises 200 mg of NMD670. In exemplary embodiments, the solid dosage form comprises 250 mg of NMD670. In exemplary embodiments, the solid dosage form comprises 300 mg of NMD670. In exemplary embodiments, the solid dosage form comprises 350 mg of NMD670. In exemplary embodiments, the solid dosage form comprises 400 mg of NMD670.

In exemplary embodiment, the composition is a solid dosage form. In exemplary embodiments, the solid dosage form is selected from the group consisting of capsule (such as sprinkle capsule and gelatine capsule), tablet (such as uncoated tablet, coated tablet, slow-release tablet) and sprinkle. In exemplary embodiments, the composition is in the form of a liquid, liquid suspension, oil, emulsion, or syrup. In exemplary embodiments, the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.

### Methods

In one aspect, the present disclosure relates to a method of treatment of spinal muscular atrophy in a subject, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.

In exemplary embodiments, the methods for treating a patient suffering from symptoms of spinal muscular atrophy may result in an increase in the total distance walked in a 6-minute walk test, an increase in muscle strength (wherein an increase can be determined by measuring grip strength, elbow flexor/extensor strength, knee flexor strength, and/or shoulder abduction strength using a handheld dynamometer), an increase in the revised Hammersmith scale score, an improvement in endurance (wherein an improvement is determined as an increase in time before dropping-out in the endurance shuttle nine hole peg test or a reduction in drop-out rate in the endurance shuttle nine hole peg test), a reduction in fatigue (wherein a reduction is a lowering of the fatigue index), an improvement in neuromuscular junction transmission (wherein an improvement is a reduction of jitter and/or blocking when measured using sfEMG), an increase in the motor function measure 32-item score, a decrease in the Fatigue Severity Scale score, a decrease in the Individualised Neuromuscular Quality of Life score, an increase in the Children's Hospital of Philadelphia Infant Test of Neuromuscular Disorders score, an increase in the Hammersmith Functional Motor Scale score, an increase in the expanded Hammersmith Functional Motor Scale score, an increase in the Hammersmith Infant Neurological Examination score, an increase in the PedsQL Neuromuscular Model score, an increase in the Patient Reported Outcomes Measurement Information System score, an increase in the Revised Upper Limb Module score, and/or an increase in the WHO Multicentre Growth Reference Study score.

Accordingly, one aspect of the present disclosure relates to methods for treating spinal muscular atrophy that result in an increase in the total distance walked in a patient, these methods comprising administering a therapeutically effective dose of NMD670 to the patient, wherein the therapeutic dose is within the range of 100 mg to 1500 mg. An increase in the total distance walked can be determined using a 6-minute walk test (ATS, 2002).

One aspect of the present disclosure relates to methods for treating spinal muscular atrophy that result in an increase in muscle strength, these methods comprising administering a therapeutically effective dose of NMD670 to the patient, wherein the therapeutic dose is within the range of 100 mg to 1500 mg. An increase in muscle strength can be determined by measuring the strength of the thigh (knee flexors), the upper arm (elbow flexor and extension) and/or the shoulder (shoulder abduction) or by measuring muscle strength using a handheld dynamometer (Febrer et al, 2010; Merlini et al, 2002).

One aspect of the present disclosure relates to methods for treating spinal muscular atrophy that result in an increase in the revised Hammersmith scale score, these methods comprising administering a therapeutically effective dose of NMD670 to the patient, wherein the therapeutic dose is within the range of 100 mg to 1500 mg. An improvement in the revised Hammersmith scale score is an increase in score (Ramsey et al 2017).

One aspect of the present disclosure relates to methods for treating spinal muscular atrophy that result in an improvement in endurance, these methods comprising administering a therapeutically effective dose of NMD670 to the patient, wherein the therapeutic dose is within the range of 100 mg to 1500 mg. An improvement in endurance can be determined using an endurance shuttle nine hole peg test (Bartels et al, 2019; Bartels et al, 2020).

One aspect of the present disclosure relates to methods for treating spinal muscular atrophy that result in a reduction in drop-out rate in the endurance shuttle nine hole peg test, these methods comprising administering a therapeutically effective dose of NMD670 to the patient, wherein the therapeutic dose is within the range of 100 mg to 1500 mg. A reduction in drop-out rate can be determined using an endurance shuttle nine hole peg test (Bartels et al, 2020).

One aspect of the present disclosure relates to methods for treating spinal muscular atrophy that result in a reduction in fatigue, these methods comprising administering a therapeutically effective dose of NMD670 to the patient, wherein the therapeutic dose is within the range of 100 mg to 1500 mg. A reduction in fatigue can be calculated from the distance walked in the 1st minute to the distance walked in the 6th minute of a 6-minute walk test (ATS, 2002)

One aspect of the present disclosure relates to methods for treating spinal muscular atrophy that result in an increase in the motor function measure 32-item score, these methods comprising administering a therapeutically effective dose of NMD670 to the patient, wherein the therapeutic dose is within the range of 100 mg to 1500 mg. An improvement in the motor function measure 32-item score is an increase in score (Vuillerot et al 2013).

One aspect of the present disclosure relates to methods for treating spinal muscular atrophy that result in a decrease in the Fatigue Severity Scale score, these methods comprising administering a therapeutically effective dose of NMD670 to the patient, wherein the therapeutic dose is within the range of 100 mg to 1500 mg. An improvement in the Fatigue Severity Scale score is a decrease in score (Kizina et al 2020).

One aspect of the present disclosure relates to methods for treating spinal muscular atrophy that result in a decrease in the Individualised Neuromuscular Quality of Life score, these methods comprising administering a therapeutically effective dose of NMD670 to the patient, wherein the therapeutic dose is within the range of 100 mg to 1500 mg. An improvement in the Individualised Neuromuscular Quality of Life score is a decrease in score (Vincent et al 2007).

One aspect of the present disclosure relates to methods for treating spinal muscular atrophy that result in an improvement in neuromuscular junction transmission, these methods comprising administering a therapeutically effective dose of NMD670 to the patient, wherein the therapeutic dose is within the range of 100 mg to 1500 mg. An improvement in neuromuscular junction transmission can be a reduction in jitter and/or blocking and can be determined using single fibre electromyography (Sanders et al, 2019).

One aspect of the present disclosure relates to methods for treating spinal muscular atrophy that result in an increase in the Children's Hospital of Philadelphia Infant Test of Neuromuscular Disorders score, these methods comprising administering a therapeutically effective dose of NMD670 to the patient, wherein the therapeutic dose is within the range of 100 mg to 1500 mg. An improvement in the Children's Hospital of Philadelphia Infant Test of Neuromuscular Disorders score is an increase in score (Glanzman et al, 2010).

One aspect of the present disclosure relates to methods for treating spinal muscular atrophy that result in an increase in the Hammersmith Functional Motor Scale score, these methods comprising administering a therapeutically effective dose of NMD670 to the patient, wherein the therapeutic dose is within the range of 100 mg to 1500 mg. An improvement in the Hammersmith Functional Motor Scale score is an increase in score (Main et al, 2003).

One aspect of the present disclosure relates to methods for treating spinal muscular atrophy that result in an increase in the expanded Hammersmith Functional Motor Scale score, these methods comprising administering a therapeutically effective dose of NMD670 to the patient, wherein the therapeutic dose is within the range of 100 mg to 1500 mg. An improvement in the expanded Hammersmith Functional Motor Scale score is an increase in score (O'Hagen et al, 2007).

One aspect of the present disclosure relates to methods for treating spinal muscular atrophy that result in an increase in the Hammersmith Infant Neurological Examination score, these methods comprising administering a therapeutically effective dose of NMD670 to the patient, wherein the therapeutic dose is within the range of 100 mg to 1500 mg. An improvement in the Hammersmith Infant Neurological Examination score is an increase in score (De Sanctis et al, 2016).

One aspect of the present disclosure relates to methods for treating spinal muscular atrophy that result in an increase in the PedsQL Neuromuscular Model score, these methods comprising administering a therapeutically effective dose of NMD670 to the patient, wherein the therapeutic dose is within the range of 100 mg to 1500 mg. An improvement in the PedsQL Neuromuscular Model score is an increase in score (Mapi Research Trust, Lyon, France).

One aspect of the present disclosure relates to methods for treating spinal muscular atrophy that result in an increase in the Patient Reported Outcomes Measurement Information System score, these methods comprising administering a therapeutically effective dose of NMD670 to the patient, wherein the therapeutic dose is within the range of 100 mg to 1500 mg. An improvement in the Patient Reported Outcomes Measurement Information System score is an increase in score (Rodday et al, 2017).

One aspect of the present disclosure relates to methods for treating spinal muscular atrophy that result in an increase in the Revised Upper Limb Module score, these methods comprising administering a therapeutically effective dose of NMD670 to the patient, wherein the therapeutic dose is within the range of 100 mg to 1500 mg. An improvement in the Revised Upper Limb Module score is an increase in score (Mazzone et al, 2016).

One aspect of the present disclosure relates to methods for treating spinal muscular atrophy that result in an increase in the WHO Multicentre Growth Reference Study score, these methods comprising administering a therapeutically effective dose of NMD670 to the patient, wherein the therapeutic dose is within the range of 100 mg to 1500 mg. An improvement in the WHO Multicentre Growth Reference Study score is an increase in score (WHO, 2006).

In one aspect, the present disclosure relates to a method for treatment of spinal muscular atrophy in a subject in need thereof, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, at a therapeutic dose of 100 to 1500 mg of (2S)-2-[4-bromo-2-(1,2-oxazol-3-yl)phenoxy]propanoic acid to said subject.

In one aspect, the present disclosure relates to a method for enhancing neuromuscular transmission and/or restoration of skeletal muscle function, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, at a therapeutic dose of 100 to 1500 mg of (2S)-2-[4-bromo-2-(1,2-oxazol-3-yl)phenoxy]propanoic acid to said subject.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered one time daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered one time daily.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered two times daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered two times daily.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered three times daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered three times daily.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered four times daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 100 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 100 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 100 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 150 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 150 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 150 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 200 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 200 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 200 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 200 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 250 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 250 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 250 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 250 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 300 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 300 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 300 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 300 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 350 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 350 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 350 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 350 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 400 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 400 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 400 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 400 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 500 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 500 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 500 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 600 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 600 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 600 mg of (2S)-2-[4-bromo-2-(1,2-oxazol-3-yl)phenoxy]propanoic acid and the composition is to be administered three times daily.

The methods of treatment disclosed herein may further comprise the administration of additional active agents that are known to treat, prevent and/or ameliorate neuromuscular disorders. These additional active agents may be agents to enhance correct splicing of SMN2 and/or to enhance SMN2 splicing.

Recently approved therapies include an antisense oligonucleotide (ASO) to enhance correct splicing of SMN2 (such as nusinersen), gene therapy (such as onasemnogene abeparvovec-xioi) to provide an alternative source the SMN1 gene leading to increased SMN protein production in patients typically up to the age of 2 years old, and a small molecule to enhance SMN2 splicing (such as risdiplam).

In one aspect, the present disclosure relates to a method for treatment of spinal muscular atrophy comprising administering (2S)-2-[4-bromo-2-(1,2-oxazol-3-yl)phenoxy]propanoic acid, or a pharmaceutically acceptable salt, hydrate, polymorph, tautomer, or solvate thereof, and an antisense oligonucleotide (ASO) to enhance correct splicing of SMN2 to a subject in need thereof. In one exemplary embodiment, antisense oligonucleotide (ASO) to enhance correct splicing of SMN2 is nusinersen or BIIB115.

In one aspect, the present disclosure relates to a method for treatment of spinal muscular atrophy comprising administering (2S)-2-[4-bromo-2-(1,2-oxazol-3-yl)phenoxy]propanoic acid, or a pharmaceutically acceptable salt, hydrate, polymorph, tautomer, or solvate thereof, and a compound to enhance SMN2 splicing to a subject in need thereof. In one exemplary embodiment, the compound to enhance SMN2 splicing is risdiplam.

In one aspect, the present disclosure relates to a method for treatment of spinal muscular atrophy comprising administering (2S)-2-[4-bromo-2-(1,2-oxazol-3-yl)phenoxy]propanoic acid, or a pharmaceutically acceptable salt, hydrate, polymorph, tautomer, or solvate thereof, and a myostatin inhibitor. In one exemplary embodiment, the myostatin inhibitor is Apitegromab. In one exemplary embodiment, the myostatin inhibitor is GYM329. In one exemplary embodiment, the myostatin inhibitor is taldefgrobep.

In some exemplary embodiments, the methods of treatment comprise administering the therapeutically effective dose of NMD670 with one or more of the following compounds: risdiplam, nusinersen, BIIB115, Salbutamol, GYM329, SRK-015, branaplam, tirasemtiv, reldesemtiv, talditercept alfa, taldefgrobep alfa, pyridostigmine, RG-6237, Apitegromab, amifampridine, nipocalimab, EXG-001307, and/or ACTX-401.

In exemplary embodiments, the methods of treatment comprise administering the therapeutically effective dose of NMD670 and the additional active agent at the same time to the patient. In other exemplary embodiments, the therapeutically effective dose of NMD670 and the additional active agent are administered at different times to the patient. In some embodiments, the therapeutically effective dose of NMD670 and the additional active agent are administered sequentially.

In one aspect, the present disclosure relates to a method of treating spinal muscular atrophy that results in an increase in total distance walked in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the total distance walked when determined using a 6-minute walk test.

In exemplary embodiments, the patient experiences an increase in total distance walked of at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 400%, such as between 5% and 200%, such as between 10% and 200%. In exemplary embodiments, the patient experiences an increase in total distance walked of at least 20 metres, such as at least 30 metres, such as at least 40 metres, such as at least 50 metres, such as at least 60 metres, such as at least 80 metres, such as at least 100 metres, such as at least 150 metres, such as at least 200 metres, such as at least 250 metres, such as at least 300 metres, such as between 20 and 400 metres, such as between 30 and 300 metres, such as between 40 and 200 metres.

In one aspect, the present disclosure relates to a method of treating spinal muscular atrophy that results in an increase in muscle strength in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in muscle strength when determined using a handheld dynamometer.

In exemplary embodiments, the patient experiences an increase in muscle strength of at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 10% and 400%, such as between 15% and 200%, such as between 20% and 100%.

In one aspect, the present disclosure relates to a method of treating spinal muscular atrophy that results in an increase in muscle strength in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in muscle strength when determined by measuring hand grip strength using a handheld dynamometer, wherein hand grip strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In one aspect, the present disclosure relates to a method of treating spinal muscular atrophy that results in an increase in muscle strength in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in muscle strength when determined by measuring knee flexor strength using a handheld dynamometer, wherein knee flexor strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as at least 5.0 kg, such as at least 7.5 kg, such as between 0.25 and 15.0 kg, such as between 0.25 and 10.0 kg, such as between 0.5 and 5.0 kg.

In one aspect, the present disclosure relates to a method of treating spinal muscular atrophy that results in an increase in muscle strength in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in muscle strength when determined by measuring elbow flexor strength using a handheld dynamometer, wherein elbow flexor strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In one aspect, the present disclosure relates to a method of treating spinal muscular atrophy that results in an increase in the revised Hammersmith scale score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the revised Hammersmith scale score.

In exemplary embodiments, the patient experiences an increase in the revised Hammersmith scale score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In one aspect, the present disclosure relates to a method of treating spinal muscular atrophy that results in an improvement in endurance in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an improvement in endurance when determined using an endurance shuttle nine hole peg test.

In exemplary embodiments, the patient experiences an improvement in endurance of at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 400%, such as between 10% and 300%, such as between 10% and 200%.

In one aspect, the present disclosure relates to a method of treating spinal muscular atrophy that results in a reduction in drop-out rate in the endurance shuttle nine hole peg test in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a reduction in drop-out rate in the endurance shuttle nine hole peg test.

In exemplary embodiments, the patient experiences a reduction in drop-out rate of at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 100%, such as between 10% and 80%, such as between 5% and 60%.

In one aspect, the present disclosure relates to a method of treating spinal muscular atrophy that results in a reduction in fatigue in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a reduction in fatigue when determined using a fatigue index.

In exemplary embodiments, the patient experiences a reduction in fatigue of at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.

In one aspect, the present disclosure relates to a method of treating spinal muscular atrophy that results in an increase in the motor function measure 32-item score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the motor function measure 32-item score.

In exemplary embodiments, the patient experiences an increase in the motor function measure 32-item score of at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.

In one aspect, the present disclosure relates to a method of treating spinal muscular atrophy that results in a decrease in the Fatigue Severity Scale score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the Fatigue Severity Scale score.

In exemplary embodiments, the patient experiences a decrease in the Fatigue Severity Scale score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In one aspect, the present disclosure relates to a method of treating spinal muscular atrophy that results in a decrease in the Individualised Neuromuscular Quality of Life score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the Individualised Neuromuscular Quality of Life score.

In exemplary embodiments, the patient experiences a decrease in the Individualised Neuromuscular Quality of Life score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In one aspect, the present disclosure relates to a method of treating spinal muscular atrophy that results in reduction in jitter in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences reduction in jitter when determined using single fibre electromyography.

In exemplary embodiments, the patient experiences a reduction in jitter of at least 15%, such as at least 10%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the patient experiences a reduction in jitter of at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs, such as between 5 μs and 200 μs, such as between 5 μs and 100 μs, such as between 10 μs and 50 μs.

In one aspect, the present disclosure relates to a method of treating spinal muscular atrophy that results in reduction in blocking in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences reduction in blocking when determined using single fibre electromyography.

In exemplary embodiments, the patient experiences a reduction in block of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In one aspect, the present disclosure relates to a method of treating spinal muscular atrophy that results in an increase in the Children's Hospital of Philadelphia Infant Test of Neuromuscular Disorders score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the Children's Hospital of Philadelphia Infant Test of Neuromuscular Disorders score.

In exemplary embodiments, the patient experiences an increase in the Children's Hospital of Philadelphia Infant Test of Neuromuscular Disorders score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points

In one aspect, the present disclosure relates to a method of treating spinal muscular atrophy that results in an increase in the Hammersmith Functional Motor Scale score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the Hammersmith Functional Motor Scale score.

In exemplary embodiments, the patient experiences an increase in the Hammersmith Functional Motor Scale score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points

In one aspect, the present disclosure relates to a method of treating spinal muscular atrophy that results in an increase in the expanded Hammersmith Functional Motor Scale score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the expanded Hammersmith Functional Motor Scale score.

In exemplary embodiments, the patient experiences an increase in the expanded Hammersmith Functional Motor Scale score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points

In one aspect, the present disclosure relates to a method of treating spinal muscular atrophy that results in an increase in the Hammersmith Infant Neurological Examination score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the Hammersmith Infant Neurological Examination score.

In exemplary embodiments, the patient experiences an increase in the Hammersmith Infant Neurological Examination score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points

In one aspect, the present disclosure relates to a method of treating spinal muscular atrophy that results in an increase in the PedsQL Neuromuscular Model score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the PedsQL Neuromuscular Model score.

In exemplary embodiments, the patient experiences an increase in the PedsQL Neuromuscular Model score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points

In one aspect, the present disclosure relates to a method of treating spinal muscular atrophy that results in an increase in the Patient Reported Outcomes Measurement Information System score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the Patient Reported Outcomes Measurement Information System score.

In exemplary embodiments, the patient experiences an increase in Patient Reported Outcomes Measurement Information System score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points

In one aspect, the present disclosure relates to a method of treating spinal muscular atrophy that results in an increase in the Revised Upper Limb Module score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the Revised Upper Limb Module score.

In exemplary embodiments, the patient experiences an increase in Revised Upper Limb Module score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points

In one aspect, the present disclosure relates to a method of treating spinal muscular atrophy that results in an increase in the WHO Multicentre Growth Reference Study score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in the WHO Multicentre Growth Reference Study score.

In exemplary embodiments, the patient experiences an increase in WHO Multicentre Growth Reference Study score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points

In exemplary embodiments, the therapeutically effective dose of the compound further provides an AUC_{f} in the range of 16,700 ng/mL to 534,000 ng/mL in the patient. In exemplary embodiments, the therapeutically effective dose of the compound has a Tₘₐₓ in the patient ranging from 1 to 6 hours. In exemplary embodiments, the therapeutically effective dose of the compound has a half-life in the patient ranging from 3 hours to 7 hours. In exemplary embodiments, the therapeutically effective dose of the compound is administered orally to the patient.

In exemplary embodiments, the subject has been diagnosed with SMA Type 0. In exemplary embodiments, the subject has been diagnosed with SMA Type 1. In exemplary embodiments, the subject has been diagnosed with SMA Type 2. In exemplary embodiments, the subject has been diagnosed with SMA Type 3. In exemplary embodiments, the subject has been diagnosed with SMA Type 4.

In exemplary embodiments, the therapeutically effective dose of the compound is administered orally to the patient. In exemplary embodiments, the therapeutically effective dose is within the range of 100 mg to 600 mg. In exemplary embodiments, the therapeutically effective dose is within the range of 200 mgs to 600 mgs. In exemplary embodiments, the therapeutically effective dose is 100 mg. In exemplary embodiments, the therapeutically effective dose is 150 mg. In exemplary embodiments, the therapeutically effective dose is 200 mg. In exemplary embodiments, the therapeutically effective dose is 250 mg. In exemplary embodiments, the therapeutically effective dose is 300 mg. In exemplary embodiments, the therapeutically effective dose is 350 mg. In exemplary embodiments, the therapeutically effective dose is 400 mg. In exemplary embodiments, the therapeutically effective dose is 500 mg. In exemplary embodiments, the therapeutically effective dose is 600 mg. In exemplary embodiments, the therapeutically effective dose is administered once, twice, three times or four times daily.

In one aspect, the present disclosure relates to a method for treating a patient suffering from symptoms of spinal muscular atrophy, the method comprising administering a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg.

In one aspect, the present disclosure relates to the use of a composition comprising (2S)-2-[4-bromo-2-(1,2-oxazol-3-yl)phenoxy]propanoic acid, or a pharmaceutically acceptable salt, hydrate, polymorph, tautomer, or solvate thereof, in the manufacture of a medicament for treatment of spinal muscular atrophy in a subject, wherein the composition is for administration at a therapeutic dose of 100 to 1500 mg of (2S)-2-[4-bromo-2-(1,2-oxazol-3-yl)phenoxy]propanoic acid.

The methods of the present disclosure may further comprise administering a second therapeutically effective dose of NMD670 to the patient either one day, two days, three days, four days, five days, six days or at least seven days after a first therapeutically effective dose is administered. The second therapeutically effective dose of NMD670 can range from 100 mg to about 1500 mg. In exemplary embodiments, the second therapeutically effective dose of NMD670 is any dosage disclosed herein. In other embodiments, the second therapeutically effective dose of NMD670 is the same as the first therapeutically effective dose administered to the patient.

In other exemplary embodiments, the methods of the present disclosure further comprise administering a third therapeutically effective dose of NMD670 to the patient either one day, two days, three days, four days, five days, six days or at least seven days after the second therapeutically effective dose has been administered. The third therapeutically effective dose of NMD670 can range from 100 mg to about 1500 mg. In exemplary embodiments, the third therapeutically effective dose of NMD670 is any dosage disclosed herein. In other embodiments, the third therapeutically effective dose of NMD670 is the same as the first therapeutically effective dose and/or the second therapeutically effective dose administered to the patient.

In some exemplary embodiments, the administration of the therapeutically effective doses of NMD670 are repeated at least 1, 2, 3, 4, 5 or 6 times weekly. In other exemplary embodiments, the administration is repeated at least 1-3 times weekly, 2-5 times weekly or 3-6 times weekly.

In some exemplary embodiments, the administration of the therapeutically effective doses of NMD670 are repeated daily. The administration of the therapeutically effective doses of NMD670 may for example be repeated 1, 2, 3, 4, 5, 6, 7 or 8 times daily. In other embodiments, the administration is repeated 1 to 8 times daily or 2 to 5 times daily.

In some embodiments, the therapeutically effective dose of NMD670 is administered at least one time daily. In exemplary embodiments, the therapeutically effective dose of NMD670 is administered one time daily.

In other embodiments, the therapeutically effective dose of NMD670 is administered either two times daily, three times daily, or four times daily.

In exemplary embodiments, the therapeutically effective dose of NMD670 is 100 to 600 mg, 300 to 500 mg, or about 400 mg and is administered one time daily. In exemplary embodiments, the therapeutically effective dose of NMD670 is 200 to 600 mg, 300 to 500 mg, or about 400 mg and is administered one time daily.

In other exemplary embodiments, the therapeutically effective dose of NMD670 is 100 to 600 mg, 300 to 500 mg, or about 400 mg and is administered two times daily. In other exemplary embodiments, the therapeutically effective dose of NMD670 is 200 to 600 mg, 300 to 500 mg, or about 400 mg and is administered two times daily. In other exemplary embodiments, the therapeutically effective dose of NMD670 is 100 to 600 mg, 300 to 500 mg, or about 400 mg and is administered three times daily. In other exemplary embodiments, the therapeutically effective dose of NMD670 is 200 to 600 mg, 300 to 500 mg, or about 400 mg and is administered three times daily.

In other exemplary embodiments, the therapeutically effective dose of NMD670 is 100 to 600 mg, 300 to 500 mg, or about 400 mg and is administered four times daily. In other exemplary embodiments, the therapeutically effective dose of NMD670 is 200 to 600 mg, 300 to 500 mg, or about 400 mg and is administered four times daily.

In some exemplary embodiments, the therapeutically effective dose of NMD670 is the daily dosage amount of NMD670. In these embodiments, the daily dosage amount of NMD670 can either be administered as a single dosage or can be administered in smaller dosages throughout the day. That is, in some embodiments the daily dosage of NMD670 is administered either once a day or at least one time daily, administered twice a day or at least at two different time points throughout the day, or administered three times a day or at least at three different time points throughout the day.

In other exemplary embodiments, the patient being administered the therapeutically effective dose of NMD670 does not have hyperuricemia. For example, the patient being administered the therapeutically effective dose of NMD670 has a level of serum uric acid below 6.5 mg/dL. Patients with serum uric acid levels above 6.5 mg/dL may not be suitable to receive the therapeutically effective dose. In exemplary embodiments wherein the patient possesses a serum uric acid level above 6.5 mg/dL, the treatment method may further comprise a step of administering a low dose of NMD670 until the patient's serum uric acid level falls below 6.5 mg/dL. A low dose of NMD670 can be from 20 mg to 150 mg, such as 25 mg to 100 mg, such as 25 mg to 50 mg. Once the patient's serum uric acid level falls below 6.5 mg/dL, they can begin receiving the therapeutically effective dose of NMD670.

### Kit-of-Parts

In one aspect, the present disclosure relates to a kit-of-parts comprising:
(2S)-2-[4-bromo-2-(1,2-oxazol-3-yl)phenoxy]propanoic acid, or a pharmaceutically acceptable salt, hydrate, polymorph, tautomer, or solvate thereof, and
a compound that increases the amount of functional SMN protein produced by SMN2.

In exemplary embodiments, the kit-of-parts comprises 100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof. In one embodiment, the compound that increases the amount of functional SMN protein produced by SMN2 is risdiplam. In one embodiment, the compound that increases the amount of functional SMN protein produced by SMN2 is nusinersen. In one embodiment, the compound that increases the amount of functional SMN protein produced by SMN2 is branaplam. In one embodiment, the compound that increases the amount of functional SMN protein produced by SMN2 is BIIB115.

In exemplary embodiments, the kit-of-parts comprises:
100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof, and
0.2 to 5 mg risdiplam, or a pharmaceutically acceptable salt, hydrate, polymorph, tautomer, or solvate thereof.

In exemplary embodiments, the kit-of-parts comprises:
100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof, and
12 to 60 mg nusinersen, or a pharmaceutically acceptable salt, hydrate, polymorph, tautomer, or solvate thereof.

In one aspect, the kit-of-parts is for use in a method of treatment of spinal muscular atrophy in a subject.

In one aspect, the present disclosure relates to (2S)-2-[4-bromo-2-(1,2-oxazol-3-yl)phenoxy]propanoic acid, or a pharmaceutically acceptable salt, hydrate, polymorph, tautomer, or solvate thereof, and a compound that increases the amount of functional SMN protein produced by SMN2 for use in the treatment of spinal muscular atrophy.

In one aspect, the kit-of-parts is for use in a method of treatment of spinal muscular atrophy in a subject. In exemplary embodiments, the kit-of-parts is for use in a method of treatment of spinal muscular atrophy in a subject, wherein the composition is for administration at a therapeutic dose of 100 to 1500 mg of (2S)-2-[4-bromo-2-(1,2-oxazol-3-yl)phenoxy]propanoic acid. In one aspect, the kit-of-parts is for use in the treatment of spinal muscular atrophy in a subject.

In one aspect, the present disclosure relates to a method for treatment of spinal muscular atrophy comprising administering (2S)-2-[4-bromo-2-(1,2-oxazol-3-yl)phenoxy]propanoic acid, or a pharmaceutically acceptable salt, hydrate, polymorph, tautomer, or solvate thereof, and a compound that increases the amount of functional SMN protein produced by SMN2 to a subject in need thereof.

In one aspect, the present disclosure relates to use of a kit-of-parts or a composition comprising (2S)-2-[4-bromo-2-(1,2-oxazol-3-yl)phenoxy]propanoic acid, or a pharmaceutically acceptable salt, hydrate, polymorph, tautomer, or solvate thereof, and a compound that increases the amount of functional SMN protein produced by SMN2 for the manufacture of a medicament for the treatment of spinal muscular atrophy.

In one aspect, the present disclosure relates to a kit-of-parts comprising:
(2S)-2-[4-bromo-2-(1,2-oxazol-3-yl)phenoxy]propanoic acid, or a pharmaceutically acceptable salt, hydrate, polymorph, tautomer, or solvate thereof, and
a myostatin inhibitor.

In one exemplary embodiment, the myostatin inhibitor is Apitegromab. In one exemplary embodiment, the myostatin inhibitor is GYM329. In one exemplary embodiment, the myostatin inhibitor is taldefgrobep.

In other exemplary embodiments, kit-of-parts further comprises an agent for increasing the Ca²⁺ sensitivity of the contractile filaments in muscle. Such an agent can, for example, be tirasemtiv or reldesemtiv.

In other exemplary embodiments, kit-of-parts further comprises one or more additional compounds, such as salbutamol, GYM329, SRK-015, talditercept alfa, taldefgrobep alfa, pyridostigmine, RG-6237, Apitegromab, amifampridine, nipocalimab, EXG-001307, and/or ACTX-401.

### Items

1. A composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein the composition is for administration at a therapeutic dose of 100 to 1500 mg of (2S)-2-[4-bromo-2-(1,2-oxazol-3-yl)phenoxy]propanoic acid.
2. The composition for use according to item 1, wherein the therapeutic dose is less than 1500 mg, such as less than 1450 mg, such as less than 1300 mg, such as less than 1250 mg, such as less than 1200 mg, such as less than 1150 mg, such as less than 1100 mg, such as less than 1050 mg, such as less than 1000 mg, such as less than 950 mg, such as less than 900 mg, such as less than 850 mg, such as less than 800 mg, such as less than 750 mg, such as less than 700 mg, such as less than 650 mg, such as less than 600 mg, such as less than 550 mg, such as less than 500 mg, such as less than 450 mg, such as less than 400 mg, such as less than 350 mg, such as less than 300 mg, such as less than 250 mg.
3. The composition for use according to any one of the preceding items, wherein the therapeutic dose is at least 100 mg, such as at least 150 mg, such as at least 200 mg, such as at least 250 mg, such as at least 300 mg, such as at least 350 mg, such as at least 400 mg, such as at least 450 mg, such as at least 500 mg, such as at least 550 mg, such as at least 600 mg, such as at least 650 mg, such as at least 700 mg, such as at least 750 mg, such as at least 800 mg, such as at least 850 mg, such as at least 900 mg, such as at least 950 mg, such as at least 1000 mg, such as at least 1050 mg, such as at least 1100 mg, such as at least 1150 mg, such as at least 1200 mg, such as at least 1250 mg, such as at least 1300 mg, such as at least 1350 mg, such as at least 1400 mg, such as at least 1450 mg.
4. The composition for use according to item 1, wherein the therapeutic dose is 200 to 600 mg, such as 250 to 550 mg, such as 300 to 500 mg, such as 350 to 450 mg, such as 375 to 425 mg, such as 400 mg.
5. The composition for use according to item 1, wherein the therapeutic dose is 700 to 1400 mg, such as 800 to 1350 mg, such as 900 to 1300 mg, such as 1000 to 1250 mg, such as 1100 to 1250 mg, such as about 1200 mg.
6. The composition for use according to item 1, wherein the therapeutic dose is about 100 mg.
7. The composition for use according to item 1, wherein the therapeutic dose is about 150 mg.
8. The composition for use according to item 1, wherein the therapeutic dose is about 200 mg.
9. The composition for use according to item 1, wherein the therapeutic dose is about 250 mg.
10. The composition for use according to item 1, wherein the therapeutic dose is about 300 mg.
11. The composition for use according to item 1, wherein the therapeutic dose is about 350 mg.
12. The composition for use according to item 1, wherein the therapeutic dose is about 400 mg.
13. The composition for use according to item 1, wherein the therapeutic dose is about 500 mg.
14. The composition for use according to item 1, wherein the therapeutic dose is about 600 mg.
15. The composition for use according to any one of the preceding items, wherein the therapeutic dose is to be administered at least one time daily.
16. The composition for use according to any one of the preceding items, wherein the therapeutic dose is to be administered one time daily.
17. The composition for use according to any one of items 1 to 15, wherein the therapeutic dose is to be administered two times daily.
18. The composition for use according to any one of items 1 to 15, wherein the therapeutic dose is to be administered three times daily.
19. The composition for use according to any one of items 1 to 15, wherein the therapeutic dose is to be administered four times daily.
20. The composition for use according to item 1, wherein the therapeutic dose is 100 to 600 mg and the composition is to be administered once time daily 21. The composition for use according to item 1, wherein the therapeutic dose is 200 to 600 mg and the composition is to be administered one time daily.
22. The composition for use according to item 1, wherein the therapeutic dose is 300 to 500 mg and the composition is to be administered one time daily.
23. The composition for use according to item 1, wherein the therapeutic dose is about 100 mg and the composition is to be administered one time daily.
24. The composition for use according to item 1, wherein the therapeutic dose is about 150 mg and the composition is to be administered one time daily.
25. The composition for use according to item 1, wherein the therapeutic dose is about 200 mg and the composition is to be administered one time daily.
26. The composition for use according to item 1, wherein the therapeutic dose is about 250 mg and the composition is to be administered one time daily.
27. The composition for use according to item 1, wherein the therapeutic dose is about 300 mg and the composition is to be administered one time daily.
28. The composition for use according to item 1, wherein the therapeutic dose is about 350 mg and the composition is to be administered one time daily.
29. The composition for use according to item 1, wherein the therapeutic dose is about 400 mg and the composition is to be administered one time daily.
30. The composition for use according to item 1, wherein the therapeutic dose is about 500 mg and the composition is to be administered one time daily.
31. The composition for use according to item 1, wherein the therapeutic dose is about 600 mg and the composition is to be administered one time daily.
32. The composition for use according to item 1, wherein the therapeutic dose is 100 to 600 mg and the composition is to be administered two times daily.
33. The composition for use according to item 1, wherein the therapeutic dose is 200 to 600 mg and the composition is to be administered two times daily.
34. The composition for use according to any one of the preceding items, wherein the therapeutic dose is 300 to 500 mg and the composition is to be administered two times daily.
35. The composition for use according to item 1, wherein the therapeutic dose is about 100 mg and the composition is to be administered two times daily.
36. The composition for use according to item 1, wherein the therapeutic dose is about 150 mg and the composition is to be administered two times daily.
37. The composition for use according to item 1, wherein the therapeutic dose is about 200 mg and the composition is to be administered two times daily.
38. The composition for use according to item 1, wherein the therapeutic dose is about 250 mg and the composition is to be administered two times daily.
39. The composition for use according to item 1, wherein the therapeutic dose is about 300 mg and the composition is to be administered two times daily.
40. The composition for use according to item 1, wherein the therapeutic dose is about 350 mg and the composition is to be administered two times daily.
41. The composition for use according to item 1, wherein the therapeutic dose is about 400 mg and the composition is to be administered two times daily.
42. The composition for use according to item 1, wherein the therapeutic dose is about 500 mg and the composition is to be administered two times daily.
43. The composition for use according to item 1, wherein the therapeutic dose is about 600 mg and the composition is to be administered two times daily.
44. The composition for use according to item 1, wherein the therapeutic dose is 100 to 600 mg and the composition is to be administered three times daily.
45. The composition for use according to item 1, wherein the therapeutic dose is 200 to 600 mg and the composition is to be administered three times daily.
46. The composition for use according to item 1, wherein the therapeutic dose is 300 to 500 mg and the composition is to be administered three times daily.
47. The composition for use according to item 1, wherein the therapeutic dose is about 100 mg and the composition is to be administered three times daily.
48. The composition for use according to item 1, wherein the therapeutic dose is about 150 mg and the composition is to be administered three times daily.
49. The composition for use according to item 1, wherein the therapeutic dose is about 200 mg and the composition is to be administered three times daily.
50. The composition for use according to item 1, wherein the therapeutic dose is about 250 mg and the composition is to be administered three times daily.
51. The composition for use according to item 1, wherein the therapeutic dose is about 300 mg and the composition is to be administered three times daily.
52. The composition for use according to item 1, wherein the therapeutic dose is about 350 mg and the composition is to be administered three times daily.
53. The composition for use according to item 1, wherein the therapeutic dose is about 400 mg and the composition is to be administered three times daily.
54. The composition for use according to item 1, wherein the therapeutic dose is about 500 mg and the composition is to be administered three times daily.
55. The composition for use according to item 1, wherein the therapeutic dose is about 600 mg and the composition is to be administered three times daily.
56. The composition for use according to item 1, wherein the therapeutic dose is 100 to 600 mg and the composition is to be administered four times daily.
57. The composition for use according to item 1, wherein the therapeutic dose is 200 to 600 mg and the composition is to be administered four times daily.
58. The composition for use according to any one of items 1 to 15, wherein the therapeutic dose is the total daily dosage.
59. The composition for use according to any one of the preceding items, wherein the composition is administered orally, parenterally, intravenously, inhaled, topically, enterally, rectally, buccally or as an aerosol.
60. The composition for use according to any one of items 1 to 59, wherein the composition is in the form of a liquid, liquid suspension, oil, emulsion, or syrup.
61. The composition for use according to any one of items 1 to 59, wherein the composition is a solid dosage form.
62. The composition for use according to item 61, wherein the solid dosage form is administered orally.
63. The composition for use according to any one of items 1 to 62, wherein the composition is in the form of a solid dosage form and is to be dosed orally, the therapeutic dose is 100 to 600 mg and the composition is to be administered one time daily.
64. The composition for use according to any one of items 1 to 62, wherein the composition is in the form of a solid dosage form and is to be dosed orally, the therapeutic dose is 200 to 600 mg and the composition is to be administered one time daily.
65. The composition for use according to any one of items 1 to 62, wherein the composition is in the form of a solid dosage form and is to be dosed orally, the therapeutic dose is 100 to 600 mg and the composition is to be administered two times daily.
66. The composition for use according to any one of items 1 to 62, wherein the composition is in the form of a solid dosage form and is to be dosed orally, the therapeutic dose is 200 to 600 mg and the composition is to be administered two times daily.
67. The composition for use according to any one of items 1 to 62, wherein the composition is in the form of a solid dosage form and is to be dosed orally, the therapeutic dose is 100 to 600 mg and the composition is to be administered three times daily.
68. The composition for use according to any one of items 1 to 62, wherein the composition is in the form of a solid dosage form and is to be dosed orally, the therapeutic dose is 200 to 600 mg and the composition is to be administered three times daily.
69. The composition for use according to any one of items 1 to 62, wherein the composition is in the form of a solid dosage form and is to be dosed orally, the therapeutic dose is 100 to 600 mg and the composition is to be administered four times daily.
70. The composition for use according to any one of items 1 to 62, wherein the composition is in the form of a solid dosage form and is to be dosed orally, the therapeutic dose is 200 to 600 mg and the composition is to be administered four times daily.
71. The composition for use according to any of items 61 to 7062, wherein the solid dosage form is selected from the group consisting of capsule (such as sprinkle capsule and gelatine capsule), tablet (such as uncoated tablet, coated tablet, slow-release tablet) and sprinkle.
72. The composition for use according to any of items 61 to 71, wherein the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.
73. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 1 to 6 hours after administration.
74. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 3 to 7 hours after administration.
75. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 12,760 to 27,440 ng/mL, such as 15,000 to 25,000 ng/mL, such as 16,000 to 24,000 ng/mL, such as 16,080 to 25,125 ng/mL, such as 17,000 to 23,000 ng/mL, such as 18,000 to 22,000 ng/mL, such as 19,000 to 21,000 ng/mL, such as about 20,100 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
76. The composition for use according to item 75, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 20,100 ng/mL and the standard deviation is 7,340 ng/mL.
77. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is about 80% to about 125%, such as 80.00% to 125.00%, of 20,100 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
78. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is 66,300 to 109,100 h·ng/mL, such as 70,000 to 105,000 h·ng/mL, such as 70,160 to 109,625 h·ng/mL, such as 75,000 to 100,000 h·ng/mL, such as 80,000 to 95,000 h·ng/mL, such as 85,000 to 90,000 h·ng/mL, such as about 87,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
79. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is 87,700 ng/mL and the standard deviation is 21,400 ng/mL.
80. The composition for use according to any one of the preceding items, wherein the composition is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is about 80% to about 125%, such as 80.00% to 125.00%, of 87,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
81. The composition for use according to any of the preceding items, wherein said AUC₀₋₂₄, AUC_{infinity}, Cₘₐₓ or Tₘₐₓ is measured after administration of a single dose to a human subject suffering from spinal muscular atrophy.
82. The composition for use according to any one of the preceding items, wherein the composition further comprises at least one pharmaceutically acceptable adjuvant and/or excipient.
83. The composition for use according to item 82, wherein the pharmaceutically acceptable adjuvant and/or excipient is selected from the group consisting of filler, binder, lubricant and disintegrant.
84. The composition for use according to item 82, wherein the pharmaceutically acceptable adjuvant and/or excipient is selected from the group consisting of silicified microcrystalline cellulose, microcrystalline cellulose, maltodextrin, magnesium stearate and croscarmellose sodium.
85. The composition for use according to any of items 61 to 72, wherein the composition comprises 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof.
86. The composition for use according to any of items 61 to 84, wherein the composition comprises:
   10 to 80 wt %, such as 40 to 65 wt %, such as 50 to 60 wt %, such as 50 to 55 wt %, such as 55 to 60 wt %, such as about 53 wt %, such as 56 wt % (2S)-2-[4-bromo-2-(1,2-oxazol-3-yl)phenoxy]propanoic acid, or a pharmaceutically acceptable salt, hydrate, polymorph, tautomer, or solvate thereof.
87. The composition for use according to any of items 61 to 84, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant; and
   e. 0.25 to 5 wt %, such as 0.3 to 2.5 wt % disintegrant;
   with the proviso that the sum of the wt % of the components does not exceed 100 wt %.
88. The composition for use according to any of items 61 to 84, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate; and
   f. 0.25 to 5 wt %, such as 0.3 to 2.5 wt % Croscarmellose sodium;
   with the proviso that the sum of the wt % of the components does not exceed 100 wt %.
89. The composition for use according to any one of items 61 to 84, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant;
   e. 0.25 to 5 wt %, such as 0.3 to 2.5 wt % disintegrant; and
   f. 1 to 10 wt % film coating:
   with the proviso that the sum of the wt % of the components does not exceed 100 wt %.
90. The composition for use according to any of items 61 to 84, wherein the composition comprises or consists of:
   a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate;
   f. 0.25 to 5 wt %, such as 0.3 to 2.5 wt % Croscarmellose sodium; and
   g. 1 to 10 wt % film coating composition such as Opadry white,
   with the proviso that the sum of the wt % of the components does not exceed 100 wt %.
91. The composition for use according to any one of the preceding items, wherein the therapeutic dose is 100 to 600 mg, the composition is in the form of a solid dosage form and is to be administered orally one time daily.
92. The composition for use according to any one of the preceding items, wherein the therapeutic dose is 200 to 600 mg, the composition is in the form of a solid dosage form and is to be administered orally one time daily.
93. The composition for use according to any one of the preceding items, wherein the therapeutic dose is 100 to 600 mg, the composition is in the form of a solid dosage form and is to be administered orally two times daily.
94. The composition for use according to any one of the preceding items, wherein the therapeutic dose is 200 to 600 mg, the composition is in the form of a solid dosage form and is to be administered orally two times daily.
95. The composition for use according to any one of the preceding items, wherein the therapeutic dose is 100 to 600 mg, the composition is in the form of a solid dosage form and is to be administered orally three times daily.
96. The composition for use according to any one of the preceding items, wherein the therapeutic dose is 200 to 600 mg, the composition is in the form of a solid dosage form and is to be administered orally three times daily.
97. The composition for use according to any one of the preceding items, wherein the therapeutic dose is 100 to 600 mg, the composition is in the form of a solid dosage form and is to be administered orally four times daily.
98. The composition for use according to any one of the preceding items, wherein the therapeutic dose is 200 to 600 mg, the composition is in the form of a solid dosage form and is to be administered orally four times daily.
99. The composition for use according to any one of the preceding items, wherein the composition is administered as one or more unit dosage forms.
100. The composition for use according to any one of the preceding items, wherein the subject has a level of serum uric acid below 6.5 mg/dL.
101. The composition for use according to any one of the preceding items, wherein the subject experiences a lessening of spinal muscular atrophy symptoms.
102. The composition for use according to any one of the preceding items, wherein the subject or a group of subjects experience an increase in the total distance walked determined using a 6-minute walk test after treatment with the composition.
103. The composition for use according to item 102, wherein the increase in the total distance walked is determined by comparing the change from baseline in the total distance walked after a defined period of time of treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the total distance walked after a defined period of time of placebo treatment.
104. The composition for use according to item 103, wherein the period of time is 21 days.
105. The composition for use according to any one of the preceding items, wherein the total distance walked has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
106. The composition for use according to any one of the preceding items, wherein the total distance walked has increased by between 5% and 400%, such as between 5% and 200%, such as between 10% and 200%,
107. The composition for use according to any one of the preceding items, wherein the total distance walked has increased by at least 20 metres, such as at least 30 metres, such as at least 40 metres, such as at least 50 metres, such as at least 60 metres, such as at least 80 metres, such as at least 100 metres, such as at least 150 metres, such as at least 200 metres, such as at least 250 metres, such as at least 300 metres.
108. The composition for use according to any one of the preceding items, wherein the total distance walked has increased by between 20 and 400 metres, such as between 30 and 300 metres, such as between 40 and 200 metres.
109. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the total distance walked when determined using the 6-minute walk test.
110. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the total distance walked when determined using the 6-minute walk test.
111. The composition for use according to any one of the preceding items, wherein the subject or a group of subjects experience an increase in muscle strength after treatment with the composition.
112. The composition for use according to item 111, wherein muscle strength is measured as grip strength.
113. The composition for use according to item 112, wherein grip strength is measured using a handheld dynamometer.
114. The composition for use according to item 111, wherein muscle strength is measured as the strength of the thigh (knee flexors), the upper arm (elbow flexor and extension) and/or the shoulder (shoulder abduction).
115. The composition for use according to any one of items 111 to 114, wherein the increase muscle strength is determined by comparing the change from baseline in muscle strength after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in muscle strength after a defined period of time of placebo treatment.
116. The composition for use according to item 115, wherein the period of time is 21 days.
117. The composition for use according to any one of the preceding items, wherein muscle strength has increased by at least 5%, such as at least 10%, such as at least 15%, such as at least 20%, such as at least 25%, such as at least 30%, such as at least 50%, such as at least 75%, such as at least 100%, such as at least 150%, such as at least 200%,
118. The composition for use according to any one of the preceding items, wherein muscle strength has increased by between 10% and 400%, such as between 15% and 200%, such as between 20% and 100%,
119. The composition for use according to any one of the preceding items, wherein muscle strength determined by measuring grip strength using a handheld dynamometer has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
120. The composition for use according to any one of the preceding items, wherein muscle strength determined by measuring grip strength using a handheld dynamometer has increased by between 10% and 400%, such as between 15% and 200%, such as between 20% and 100%.
121. The composition for use according to any one of the preceding items, wherein muscle strength determined by measuring grip strength using a handheld dynamometer has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg.
122. The composition for use according to any one of the preceding items, wherein muscle strength determined by measuring grip strength using a handheld dynamometer has increased by between 0.25 and 5.0 kg, such as between 0.25 and 4.0 kg, such as between 0.5 and 4.0 kg.
123. The composition for use according to any one of the preceding items, wherein muscle strength determined by measuring knee flexor strength using a handheld dynamometer has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as at least 5.0 kg, such as at least 7.5 kg.
124. The composition for use according to any one of the preceding items, wherein muscle strength determined by measuring knee flexor strength using a handheld dynamometer has increased by between 0.25 and 15.0 kg, such as between 0.25 and 10.0 kg, such as between 0.5 and 5.0 kg.
125. The composition for use according to any one of the preceding items, wherein muscle strength determined by measuring elbow flexor strength using a handheld dynamometer has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg.
126. The composition for use according to any one of the preceding items, wherein muscle strength determined by measuring elbow flexor strength using a handheld dynamometer has increased by between 0.25 and 5.0 kg, such as between 0.25 and 4.0 kg, such as between 0.5 and 4.0 kg.
127. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in in muscle strength when determined by measuring grip strength using a handheld dynamometer.
128. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in in muscle strength when determined by measuring grip strength using a handheld dynamometer.
129. The composition for use according to any one of the preceding items, wherein the subject or a group of subjects experience an increase in the revised Hammersmith scale score after treatment with the composition.
130. The composition for use according to item 129, wherein the increase the revised Hammersmith scale score is determined by comparing the change from baseline in the revised Hammersmith scale score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the revised Hammersmith scale score after a defined period of time of placebo treatment.
131. The composition for use according to item 130, wherein the period of time is 21 days.
132. The composition for use according to any one of the preceding items, wherein the revised Hammersmith scale score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
133. The composition for use according to any one of the preceding items, wherein the revised Hammersmith scale score has increased by between 0.5 and 30 points, such as between 1 and 20 points, such as between 0.5 and 10 points.
134. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the revised Hammersmith scale score.
135. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the revised Hammersmith scale score.
136. The composition for use according to any one of the preceding items, wherein the subject or a group of subjects experience an improvement in endurance after treatment with the composition.
137. The composition for use according to item 136, wherein improvement in endurance is determined using an endurance shuttle nine hole peg test.
138. The composition for use according to item 137, wherein the increase improvement in endurance is determined by comparing the change from baseline in the endurance shuttle nine hole peg test after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the endurance shuttle nine hole peg test after a defined period of time of placebo treatment.
139. The composition for use according to item 138, wherein the period of time is 21 days.
140. The composition for use according to any one of the preceding items, wherein the endurance determined using an endurance shuttle nine hole peg test has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
141. The composition for use according to any one of the preceding items, wherein the endurance determined using an endurance shuttle nine hole peg test has increased by between 5% and 400%, such as between 10% and 300%, such as between 10% and 200%.
142. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in endurance when determined using an endurance shuttle nine hole peg test.
143. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in endurance when determined using an endurance shuttle nine hole peg test.
144. The composition for use according to any one of the preceding items, wherein the subject or a group of subjects experience a reduction in drop-out rate in the endurance shuttle nine hole peg test after treatment with the composition.
145. The composition for use according to item 137, wherein the reduction in drop-out rate in the endurance shuttle nine hole peg test is determined by comparing the change from baseline in the drop-out rate in the endurance shuttle nine hole peg test after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the drop-out rate in the endurance shuttle nine hole peg test after a defined period of time of placebo treatment.
146. The composition for use according to item 145, wherein the period of time is 21 days.
147. The composition for use according to any one of the preceding items, wherein the reduction in drop-out rate in the endurance shuttle nine hole peg test has decreased by at least 5%, such as at least 10%, such as at least 15%, such as at least 20%, such as at least 25%, such as at least 30%, such as at least 40%, such as at least 50%, such as at least 60%, such as at least 70%, such as at least 80%.
148. The composition for use according to any one of the preceding items, wherein the reduction in drop-out rate in the endurance shuttle nine hole peg test has decreased by between 5% and 100%, such as between 10% and 80%, such as between 5% and 60%.
149. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in drop-out rate in the endurance shuttle nine hole peg test.
150. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in drop-out rate in the endurance shuttle nine hole peg test.
151. The composition for use according to any one of the preceding items, wherein the subject or a group of subjects experience a reduction in fatigue after treatment with the composition.
152. The composition for use according to item 151, wherein the reduction in fatigue is determined using a fatigue index which is calculated from the distance walked in the 1st minute to the distance walked in the 6th minute of a 6-minute walk test.
153. The composition for use according to item 151, wherein the reduction in fatigue is determined by comparing the change from baseline in the fatigue index after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the drop-out rate in the fatigue index after a defined period of time of placebo treatment.
154. The composition for use according to item 153, wherein the period of time is 21 days.
155. The composition for use according to any one of the preceding items, wherein the reduction in fatigue determined using a fatigue index has decreased by at least 5%, such as at least 10%, such as at least 15%, such as at least 20%, such as at least 25%, such as at least 30%, such as at least 40%, such as at least 50%, such as at least 60%, such as at least 70%, such as at least 80%.
156. The composition for use according to any one of the preceding items, wherein the reduction in fatigue determined using a fatigue index has decreased by between 5% and 95%, such as between 5% and 80%, such as between 10% and 50%.
157. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in fatigue when determined using a fatigue index.
158. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in fatigue when determined using a fatigue index.
159. The composition for use according to any one of the preceding items, wherein the subject or a group of subjects experience an increase in the motor function measure 32-item score after treatment with the composition.
160. The composition for use according to item 159, wherein the increase in the motor function measure 32-item score is determined by comparing the change from baseline in the motor function measure 32-item score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the motor function measure 32-item score after a defined period of time of placebo treatment.
161. The composition for use according to item 160, wherein the period of time is 21 days.
162. The composition for use according to any one of the preceding items, wherein the motor function measure 32-item score has increased by at least 5%, such as at least 10%, such as at least 15%, such as at least 20%, such as at least 25%, such as at least 30%, such as at least 40%, such as at least 50%, such as at least 60%, such as at least 70%, such as at least 80%.
163. The composition for use according to any one of the preceding items, wherein the motor function measure 32-item score has increased by between 5% and 95%, such as between 5% and 80%, such as between 10% and 50%.
164. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the motor function measure 32-item score.
165. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the motor function measure 32-item score.
166. The composition for use according to any one of the preceding items, wherein the subject or a group of subjects experience a decrease in the Fatigue Severity Scale score after treatment with the composition.
167. The composition for use according to item 166, wherein the decrease in the Fatigue Severity Scale score is determined by comparing the change from baseline in the Fatigue Severity Scale score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the Fatigue Severity Scale score after a defined period of time of placebo treatment.
168. The composition for use according to item 167, wherein the period of time is 21 days.
169. The composition for use according to any one of items 166 to 168, wherein the Fatigue Severity Scale score has decreased by 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
170. The composition for use according to any one of items 166 to 169, wherein the Fatigue Severity Scale score has decreased by between 1 and 20 points, such as between 0.5 and 10 points.
171. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Fatigue Severity Scale score.
172. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Fatigue Severity Scale score.
173. The composition for use according to any one of the preceding items, wherein the subject or a group of subjects experience a decrease in the Individualised Neuromuscular Quality of Life score after treatment with the composition.
174. The composition for use according to item 173, wherein the decrease in the Individualised Neuromuscular Quality of Life score is determined by comparing the change from baseline in the Individualised Neuromuscular Quality of Life score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the Individualised Neuromuscular Quality of Life score after a defined period of time of placebo treatment.
175. The composition for use according to item 174, wherein the period of time is 21 days.
176. The composition for use according to any one of the preceding items, wherein the Individualised Neuromuscular Quality of Life score has decreased by 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
177. The composition for use according to any one of the preceding items, wherein the Individualised Neuromuscular Quality of Life score has decreased by between 0.5 and 30 points, such as between 1 and 20 points, such as between 0.5 and 10 points.
178. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Individualised Neuromuscular Quality of Life score.
179. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Individualised Neuromuscular Quality of Life score.
180. The composition for use according to any one of the preceding items, wherein the subject or a group of subjects experience a reduction in jitter after treatment with the composition.
181. The composition for use according to item 180, wherein jitter is determined using single fibre electromyography.
182. The composition for use according to any one of items 180 or 181, wherein the reduction in jitter is determined by comparing the change from baseline in jitter after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in jitter after a defined period of time of placebo treatment.
183. The composition for use according to item 182, wherein the period of time is 21 days.
184. The composition for use according to any one items 180 to 183, wherein jitter has been reduced by at least 10%, such as at least 15%, such as at least 20%, such as at least 25%, such as at least 30%, such as at least 50%, such as at least 75%, such as at least 100%, such as at least 150%, such as at least 200%.
185. The composition for use according to any one of items 180 to 183, wherein jitter has been reduced by between 5% and 95%, such as between 5% and 80%, such as between 10% and 50%.
186. The composition for use according to any one items 180 to 183, wherein jitter determined using single fibre electromyography has been reduced by at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs.
187. The composition for use according to any one items 180 to 183, wherein jitter determined using single fibre electromyography has been reduced by between 5 μs and 200 μs, such as between 5 μs and 100 μs, such as between 10 μs and 50 μs.
188. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in jitter when determined using single fibre electromyography.
189. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in jitter when determined using single fibre electromyography.
190. The composition for use according to any one of the preceding items, wherein the subject or a group of subjects experience a reduction in blocking after treatment with the composition.
191. The composition for use according to item 190, wherein blocking is determined using single fibre electromyography.
192. The composition for use according to any one of items 190 or 191, wherein the reduction in blocking is determined by comparing the change from baseline in blocking after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in blocking after a defined period of time of placebo treatment.
193. The composition for use according to item 192, wherein the period of time is 21 days.
194. The composition for use according to any one of items 190 to 193, wherein blocking has been reduced by at least 5%, such as at least 10%, such as at least 15%, such as at least 20%, such as at least 25%, such as at least 30%, such as at least 50%, such as at least 75%, such as at least 100%, such as at least 150%, such as at least 200%.
195. The composition for use according to any one of items 190 to 194, wherein blocking has been reduced by between 5% and 95%, such as between 5% and 80%, such as between 10% and 50%.
196. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in blocking when determined using single fibre electromyography.
197. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in blocking when determined using single fibre electromyography.
198. The composition for use according to any one of the preceding items, wherein the subject or a group of subjects experience an increase in the Children's Hospital of Philadelphia Infant Test of Neuromuscular Disorders (CHOP INTEND) score after treatment with the composition.
199. The composition for use according to item 198, wherein the increase in the CHOP INTEND score is determined by comparing the change from baseline in the CHOP INTEND score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in CHOP INTEND score after a defined period of time of placebo treatment.
200. The composition for use according to item 199, wherein the period of time is 21 days.
201. The composition for use according to any one items 199 to 200, wherein the CHOP INTEND score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
202. The composition for use according to any one items 199 to 201, wherein the CHOP INTEND score has increased by between 0.5 and 30 points, such as between 1 and 20 points, such as between 0.5 and 10 points.
203. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the Children's Hospital of Philadelphia Infant Test of Neuromuscular Disorders score.
204. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the Children's Hospital of Philadelphia Infant Test of Neuromuscular Disorders score.
205. The composition for use according to any one of the preceding items, wherein the subject or a group of subjects experience an increase in the Hammersmith Functional Motor Scale (HFMS) score after treatment with the composition.
206. The composition for use according to item 205, wherein the increase in the HFMS score is determined by comparing the change from baseline in the HFMS score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in HFMS score after a defined period of time of placebo treatment.
207. The composition for use according to item 206, wherein the period of time is 21 days.
208. The composition for use according to any one of items 205 to 207, wherein the HFMS score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
209. The composition for use according to any one of items 205 to 207, wherein the HFMS score has increased by between 0.5 and 30 points, such as between 1 and 20 points, such as between 0.5 and 10 points.
210. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the Hammersmith Functional Motor Scale score.
211. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the Hammersmith Functional Motor Scale score.
212. The composition for use according to any one of the preceding items, wherein the subject or a group of subjects experience an increase in the expanded Hammersmith Functional Motor Scale (HFMSE) score after treatment with the composition.
213. The composition for use according to item 212, wherein the increase in the HFMSE score is determined by comparing the change from baseline in the HFMSE score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in HFMSE score after a defined period of time of placebo treatment.
214. The composition for use according to item 213, wherein the period of time is 21 days.
215. The composition for use according to any one of items 213 or 214, wherein the HFMSE score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
216. The composition for use according to any one of items 213 or 214, wherein the HFMSE score has increased by between 0.5 and 30 points, such as between 1 and 20 points, such as between 0.5 and 10 points.
217. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the expanded Hammersmith Functional Motor Scale score.
218. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the expanded Hammersmith Functional Motor Scale score.
219. The composition for use according to any one of the preceding items, wherein the subject or a group of subjects experience an increase in the Hammersmith Infant Neurological Examination (HINE) score after treatment with the composition.
220. The composition for use according to item 219, wherein the increase in the HINE score is determined by comparing the change from baseline in the HINE score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in HINE score after a defined period of time of placebo treatment.
221. The composition for use according to item 220, wherein the period of time is 21 days.
222. The composition for use according to any one of items 219 to 221, wherein the HINE score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
223. The composition for use according to any one of items 219 to 221, wherein the HINE score has increased by between 0.5 and 30 points, such as between 1 and 20 points, such as between 0.5 and 10 points.
224. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in Infant Neurological Examination score.
225. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in Infant Neurological Examination score.
226. The composition for use according to any one of the preceding items, wherein the subject or a group of subjects experience an increase in the PedsQL Neuromuscular Model score after treatment with the composition.
227. The composition for use according to item 226, wherein the increase in the PedsQL Neuromuscular Model score is determined by comparing the change from baseline in the PedsQL Neuromuscular Model score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the PedsQL Neuromuscular Model score after a defined period of time of placebo treatment.
228. The composition for use according to item 227, wherein the period of time is 21 days.
229. The composition for use according to any one of items 226 to 228, wherein the PedsQL Neuromuscular Model score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
230. The composition for use according to any one of items 226 to 228, wherein the PedsQL Neuromuscular Model score has increased by between 0.5 and 30 points, such as between 1 and 20 points, such as between 0.5 and 10 points.
231. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the PedsQL Neuromuscular Model score.
232. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the PedsQL Neuromuscular Model score.
233. The composition for use according to any one of the preceding items, wherein the subject or a group of subjects experience an increase in the Patient Reported Outcomes Measurement Information System (PROMIS) score after treatment with the composition.
234. The composition for use according to item 233, wherein the increase in the PROMIS score is determined by comparing the change from baseline in the PROMIS score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the PROMIS score after a defined period of time of placebo treatment.
235. The composition for use according to item 234, wherein the period of time is 21 days.
236. The composition for use according to any one of items 233 to 235, wherein the PROMIS score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
237. The composition for use according to any one of items 233 to 235, wherein the PROMIS score has increased by between 0.5 and 30 points, such as between 1 and 20 points, such as between 0.5 and 10 points.
238. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the Patient Reported Outcomes Measurement Information System score.
239. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the Patient Reported Outcomes Measurement Information System score.
240. The composition for use according to any one of the preceding items, wherein the subject or a group of subjects experience an increase in the Revised Upper Limb Module (RULM) score after treatment with the composition.
241. The composition for use according to item 240, wherein the increase in the RULM score is determined by comparing the change from baseline in the RULM score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the RULM score after a defined period of time of placebo treatment.
242. The composition for use according to item 241, wherein the period of time is 21 days.
243. The composition for use according to any one of items 240 to 242, wherein the RULM score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
244. The composition for use according to any one of items 240 to 242, wherein the RULM score has increased by between 0.5 and 30 points, such as between 1 and 20 points, such as between 0.5 and 10 points.
245. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the Revised Upper Limb Module score.
246. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the Revised Upper Limb Module score.
247. The composition for use according to any one of the preceding items, wherein the subject or a group of subjects experience an increase in the WHO Multicentre Growth Reference Study (MGRS) score after treatment with the composition.
248. The composition for use according to item 247, wherein the increase in the MGRS score is determined by comparing the change from baseline in the MGRS score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the MGRS score after a defined period of time of placebo treatment.
249. The composition for use according to item 248, wherein the period of time is 21 days.
250. The composition for use according to any one of items 247 to 249, wherein the MGRS score has increased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
251. The composition for use according to any one of items 247 to 249, wherein the MGRS score has increased by between 0.5 and 30 points, such as between 1 and 20 points, such as between 0.5 and 10 points.
252. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the WHO Multicentre Growth Reference Study score.
253. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in the WHO Multicentre Growth Reference Study score.
254. A composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of spinal muscular atrophy in a subject, wherein the subject has a level of serum uric acid below 6.5 mg/dL.
255. The composition for use according to item 254, wherein the composition is for administration at a therapeutic dose of 100 to 1500 mg of (2S)-2-[4-bromo-2-(1,2-oxazol-3-yl)phenoxy]propanoic acid.
256. A method for treatment of spinal muscular atrophy in a subject, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
257. The method according to item 256, wherein the method results in:
   a) an increase in the total distance walked in a 6-minute walk test;
   b) an increase in muscle strength, for example determined by measuring grip strength, elbow flexor/extensor strength, knee flexor strength, and/or shoulder abduction strength using a handheld dynamometer;
   c) an increase in the revised Hammersmith scale score;
   d) an improvement in endurance, for example determined as an increase in time before dropping-out in the endurance shuttle nine hole peg test or a reduction in drop-out rate in the endurance shuttle nine hole peg test;
   e) a reduction in fatigue, for example a reduction is a lowering of the fatigue index;
   f) an improvement in neuromuscular junction transmission, for example a reduction of jitter and/or blocking when measured using sfEMG;
   g) an increase in the motor function measure 32-item score;
   h) a decrease in the Fatigue Severity Scale score;
   i) a decrease in the Individualised Neuromuscular Quality of Life score;
   j) an increase in the Children's Hospital of Philadelphia Infant Test of Neuromuscular Disorders score;
   k) an increase in the Hammersmith Functional Motor Scale score;
   l) an increase in the expanded Hammersmith Functional Motor Scale score;
   m) an increase in the Hammersmith Infant Neurological Examination score;
   n) an increase in the PedsQL Neuromuscular Model score;
   o) an increase in the Patient Reported Outcomes Measurement Information System score;
   p) an increase in the Revised Upper Limb Module score; and/or
   q) an increase in the WHO Multicentre Growth Reference Study score.
258. A method for treating spinal muscular atrophy in a subject that result in an increase in the total distance walked, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
259. The method according to item 258, wherein the increase in the total distance walked is determined using a 6-minute walk test.
260. The method according to item 258, wherein the subject experiences an increase in total distance walked of at least 5%, such as at least 10%, such as at least 15%, such as at least 20%, such as at least 25%, such as at least 30%, such as at least 50%, such as at least 75%, such as at least 100%, such as at least 150%, such as at least 200%.
261. The method according to any one of items 259 or 260, wherein the subject experiences an increase in total distance walked of between 5% and 400%, such as between 5% and 200%, such as between 10% and 200%.
262. The method according to any one of items 259 or 260, wherein the subject experiences an increase in total distance walked of least 20 metres, such as at least 30 metres, such as at least 40 metres, such as at least 50 metres, such as at least 60 metres, such as at least 80 metres, such as at least 100 metres, such as at least 150 metres, such as at least 200 metres, such as at least 250 metres, such as at least 300 metres.
263. The method according to any one of items 259 or 260, wherein the subject experiences an increase in total distance walked of between 20 and 400 metres, such as between 30 and 300 metres, such as between 40 and 200 metres.
264. A method for treating spinal muscular atrophy in a subject that result in an increase in muscle strength, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
265. The method according to item 264, wherein the increase in muscle strength is determined by measuring the strength of the thigh (knee flexors), the upper arm (elbow flexor and extension) and/or the shoulder (shoulder abduction) or by measuring grip strength using a handheld dynamometer.
266. The method according to any one of items 264 or 265, wherein the subject experiences an increase in muscle strength of at least 5%, such as at least 10%, such as at least 15%, such as at least 20%, such as at least 25%, such as at least 30%, such as at least 50%, such as at least 75%, such as at least 100%, such as at least 150%, such as at least 200%.
267. The method according to one of items 264 to 266, wherein the subject experiences an increase in muscle strength of between 10% and 400%, such as between 15% and 200%, such as between 20% and 100%.
268. The method according to item 264, wherein the subject experiences an increase in muscle strength when determined by measuring hand grip strength using a handheld dynamometer, wherein hand grip strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.
269. The method according to item 264, wherein the subject experiences an increase in muscle strength when determined by measuring elbow flexor strength using a handheld dynamometer, wherein elbow flexor strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.
270. The method according to item 264, wherein the subject experiences an increase in muscle strength when determined by measuring knee flexor strength using a handheld dynamometer, wherein knee flexor strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as at least 5.0 kg, such as at least 7.5 kg, such as between 0.25 and 15.0 kg, such as between 0.25 and 10.0 kg, such as between 0.5 and 5.0 kg.
271. A method for treating spinal muscular atrophy in a subject that result in an increase in the revised Hammersmith scale score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
272. The method according to item 271, wherein the therapeutically effective dose provides a Cₘₐₓ in the range of 2,790 ng/mL to 76,700 ng/mL in the subject.
273. The method according to one of items 271 or 272, wherein the subject experiences an increase in the revised Hammersmith scale score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
274. A method for treating spinal muscular atrophy in a subject that result in an improvement in endurance, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
275. The method according to item 274, wherein the improvement in endurance is determined using an endurance shuttle nine hole peg test.
276. The method according to item 275, wherein the therapeutically effective dose provides a Cₘₐₓ in the range of 2,790 ng/mL to 76,700 ng/mL in the subject.
277. The method according to one of items 274 to 276, wherein the subject experiences an improvement in endurance of at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 400%, such as between 10% and 300%, such as between 10% and 200%.
278. A method for treating spinal muscular atrophy in a subject that result in a reduction in drop-out rate in the endurance shuttle nine hole peg test, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
279. The method according to item 278, wherein the therapeutically effective dose provides a Cₘₐₓ in the range of 2,790 ng/mL to 76,700 ng/mL in the subject.
280. The method according to any one of items 278 or 279, wherein the subject experiences a reduction in drop-out rate of at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 100%, such as between 10% and 80%, such as between 5% and 60%.
281. A method for treating spinal muscular atrophy in a subject that result in a reduction in fatigue, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
282. The method according to item 281, wherein the reduction in fatigue is calculated from the distance walked in the 1st minute to the distance walked in the 6th minute of a 6-minute walk test.
283. The method according to any one of items 281 or 282, wherein the subject experiences a reduction in fatigue of at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.
284. A method for treating spinal muscular atrophy in a subject that result in an increase in the motor function measure 32-item score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
285. The method according to item 284, wherein the subject experiences an increase in the motor function measure 32-item score of at least 5%, such as at least 10%, such as at least 15%, such as at least 20%, such as at least 25%, such as at least 30%, such as at least 40%, such as at least 50%, such as at least 60%, such as at least 70%, such as at least 80%, such as between 5% and 95%, such as between 5% and 80%, such as between 10% and 50%.
286. A method for treating spinal muscular atrophy in a subject that result in a decrease in the Fatigue Severity Scale score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
287. The method according to item 286, wherein the subject experiences a decrease in the Fatigue Severity Scale score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
288. A method for treating spinal muscular atrophy in a subject that result in a decrease in the Individualised Neuromuscular Quality of Life score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
289. The method according to item 288, wherein the subject experiences a decrease in the Individualised Neuromuscular Quality of Life score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
290. A method for treating spinal muscular atrophy in a subject that result in an improvement in neuromuscular junction transmission, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
291. The method according to item 290, wherein the improvement in neuromuscular junction is a reduction in jitter and/or blocking and is determined using single fibre electromyography.
292. The method according to any one of items 290 or 291, wherein the subject experiences a reduction in jitter of at least 15%, such as at least 10%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
293. The method according to any one of items 290 or 291, wherein the subject experiences a reduction in jitter of at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs, such as between 5 μs and 200 μs, such as between 5 μs and 100 μs, such as between 10 μs and 50 μs.
294. The method according to any one of items 290 or 291, wherein the subject experiences a reduction in block of at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
295. A method for treating spinal muscular atrophy in a subject that result in an increase in the Children's Hospital of Philadelphia Infant Test of Neuromuscular Disorders score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
296. The method according to item 295, wherein the subject experiences an increase in the Children's Hospital of Philadelphia Infant Test of Neuromuscular Disorders score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
297. A method for treating spinal muscular atrophy in a subject that result in an increase in the Hammersmith Functional Motor Scale score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
298. The method according to item 297, wherein the subject experiences an increase in the Hammersmith Functional Motor Scale score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
299. A method for treating spinal muscular atrophy in a subject that result in an increase in the expanded Hammersmith Functional Motor Scale score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
300. The method according to item 299, wherein the subject experiences an increase in the expanded Hammersmith Functional Motor Scale score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
301. A method for treating spinal muscular atrophy in a subject that result in an increase in the Hammersmith Infant Neurological Examination score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
302. The method according to item 301, wherein the subject experiences an increase in the Hammersmith Infant Neurological Examination score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
303. A method for treating spinal muscular atrophy in a subject that result in an increase in the PedsQL Neuromuscular Model score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
304. The method according to item 303, wherein the subject experiences an increase in the PedsQL Neuromuscular Model score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
305. A method for treating spinal muscular atrophy in a subject that result in an increase in the Patient Reported Outcomes Measurement Information System score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
306. The method according to item 305, wherein the subject experiences an increase in Patient Reported Outcomes Measurement Information System score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
307. A method for treating spinal muscular atrophy in a subject that result in an increase in the Revised Upper Limb Module score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
308. The method according to item 307, wherein the subject experiences an increase in Revised Upper Limb Module score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
309. A method for treating spinal muscular atrophy in a subject that result in an increase in the WHO Multicentre Growth Reference Study score, said method comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
310. The method according to item 309, wherein the subject experiences an increase in WHO Multicentre Growth Reference Study score of at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
311. A method for treating a subject suffering from symptoms of spinal muscular atrophy, the method comprising administering a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg.
312. A method for treatment of spinal muscular atrophy in a subject with serum uric acid levels above 6.5 mg/dL, said method comprising administering a low dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject until the patient's serum uric acid level falls below 6.5 mg/dL, and then administering a therapeutic dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the low dose is within the range of 20 mg to 150 mg, such as 25 mg to 100 mg, such as 25 mg to 50 mg, and the therapeutic dose is within the range of 200 mg to 1500 mg.
313. A method for enhancing neuromuscular transmission and/or restoration of skeletal muscle function, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, at a therapeutic dose of 100 to 1500 mg of (2S)-2-[4-bromo-2-(1,2-oxazol-3-yl)phenoxy]propanoic acid to said subject.
314. The method according to one of items 256 to 313, wherein the therapeutically effective dose provides a Cₘₐₓ in the range of 2,790 ng/mL to 76,700 ng/mL in the subject.
315. The method according to one of items 256 to 314, wherein the therapeutically effective dose provides an AUC_{inf} in the range of 16,700 ng/mL to 534,000 ng/mL in the subject.
316. The method according to one of items 256 to 315, wherein the therapeutically effective dose of the compound has a Tₘₐₓ in the subject ranging from 1 to 6 hours.
317. The method according to one of items 256 to 315, wherein the therapeutically effective dose of the compound has a Tₘₐₓ in the subject ranging from 3 hours to 7 hours.
318. The method according to one of items 256 to 317, wherein the therapeutically effective dose is within the range of 100 mg to 600 mg.
319. The method according to one of items 256 to 317, wherein the therapeutically effective dose is within the range of 200 mg to 600 mg.
320. The method according to one of items 256 to 317, wherein the therapeutically effective dose is 100 mg.
321. The method according to one of items 256 to 317, wherein the therapeutically effective dose is 150 mg.
322. The method according to one of items 256 to 317, wherein the therapeutically effective dose is 200 mg.
323. The method according to one of items 256 to 317, wherein the therapeutically effective dose is 250 mg.
324. The method according to one of items 256 to 317, wherein the therapeutically effective dose is 300 mg.
325. The method according to one of items 256 to 317, wherein the therapeutically effective dose is 350 mg.
326. The method according to one of items 256 to 317, wherein the therapeutically effective dose is 400 mg.
327. The method according to one of items 256 to 317, wherein the therapeutically effective dose is 500 mg.
328. The method according to one of items 256 to 317, wherein the therapeutically effective dose is 600 mg.
329. The method according to one of items 256 to 328, wherein the therapeutically effective dose is administered once, twice, three times or four times daily.
330. The method according to one of items 256 to 313, wherein the therapeutically effective dose is 100 to 600 mg of (2S)-2-[4-bromo-2-(1,2-oxazol-3-yl)phenoxy]propanoic acid and the composition is to be administered one time daily.
331. The method according to one of items 256 to 313, wherein the therapeutically effective dose is 100 to 600 mg of (2S)-2-[4-bromo-2-(1,2-oxazol-3-yl)phenoxy]propanoic acid and the composition is to be administered two times daily.
332. The method according to one of items 256 to 313, wherein the therapeutically effective dose is 100 to 600 mg of (2S)-2-[4-bromo-2-(1,2-oxazol-3-yl)phenoxy]propanoic acid and the composition is to be administered three times daily.
333. The method according to one of items 256 to 313, wherein the therapeutically effective dose is 100 to 600 mg of (2S)-2-[4-bromo-2-(1,2-oxazol-3-yl)phenoxy]propanoic acid and the composition is to be administered four times daily.
334. The method according to one of items 256 to 313, wherein the therapeutically effective dose is 200 to 600 mg of (2S)-2-[4-bromo-2-(1,2-oxazol-3-yl)phenoxy]propanoic acid and the composition is to be administered one time daily.
335. The method according to one of items 256 to 313, wherein the therapeutically effective dose is 200 to 600 mg of (2S)-2-[4-bromo-2-(1,2-oxazol-3-yl)phenoxy]propanoic acid and the composition is to be administered two times daily.
336. The method according to one of items 256 to 313, wherein the therapeutically effective dose is 200 to 600 mg of (2S)-2-[4-bromo-2-(1,2-oxazol-3-yl)phenoxy]propanoic acid and the composition is to be administered three times daily.
337. The method according to one of items 256 to 313, wherein the therapeutically effective dose is 200 to 600 mg of (2S)-2-[4-bromo-2-(1,2-oxazol-3-yl)phenoxy]propanoic acid and the composition is to be administered four times daily.
338. The method according to one of items 256 to 313, wherein the therapeutically effective dose is about 100 mg of (2S)-2-[4-bromo-2-(1,2-oxazol-3-yl)phenoxy]propanoic acid and the composition is to be administered one time daily.
339. The method according to one of items 256 to 313, wherein the therapeutically effective dose is about 100 mg of (2S)-2-[4-bromo-2-(1,2-oxazol-3-yl)phenoxy]propanoic acid and the composition is to be administered two times daily.
340. The method according to one of items 256 to 313, wherein the therapeutically effective dose is about 100 mg of (2S)-2-[4-bromo-2-(1,2-oxazol-3-yl)phenoxy]propanoic acid and the composition is to be administered three times daily.
341. The method according to one of items 256 to 313, wherein the therapeutically effective dose is about 100 mg of (2S)-2-[4-bromo-2-(1,2-oxazol-3-yl)phenoxy]propanoic acid and the composition is to be administered four times daily.
342. The method according to one of items 256 to 313, wherein the therapeutically effective dose is about 150 mg of (2S)-2-[4-bromo-2-(1,2-oxazol-3-yl)phenoxy]propanoic acid and the composition is to be administered one time daily.
343. The method according to one of items 256 to 313, wherein the therapeutically effective dose is about 150 mg of (2S)-2-[4-bromo-2-(1,2-oxazol-3-yl)phenoxy]propanoic acid and the composition is to be administered two times daily.
344. The method according to one of items 256 to 313, wherein the therapeutically effective dose is about 150 mg of (2S)-2-[4-bromo-2-(1,2-oxazol-3-yl)phenoxy]propanoic acid and the composition is to be administered three times daily.
345. The method according to one of items 256 to 313, wherein the therapeutically effective dose is about 150 mg of (2S)-2-[4-bromo-2-(1,2-oxazol-3-yl)phenoxy]propanoic acid and the composition is to be administered four times daily.
346. The method according to one of items 256 to 313, wherein the therapeutically effective dose is about 200 mg of (2S)-2-[4-bromo-2-(1,2-oxazol-3-yl)phenoxy]propanoic acid and the composition is to be administered one time daily.
347. The method according to one of items 256 to 313, wherein the therapeutically effective dose is about 200 mg of (2S)-2-[4-bromo-2-(1,2-oxazol-3-yl)phenoxy]propanoic acid and the composition is to be administered two times daily.
348. The method according to one of items 256 to 313, wherein the therapeutically effective dose is about 200 mg of (2S)-2-[4-bromo-2-(1,2-oxazol-3-yl)phenoxy]propanoic acid and the composition is to be administered three times daily.
349. The method according to one of items 256 to 313, wherein the therapeutically effective dose is about 200 mg of (2S)-2-[4-bromo-2-(1,2-oxazol-3-yl)phenoxy]propanoic acid and the composition is to be administered four times daily.
350. The method according to one of items 256 to 313, wherein the therapeutically effective dose is about 250 mg of (2S)-2-[4-bromo-2-(1,2-oxazol-3-yl)phenoxy]propanoic acid and the composition is to be administered one time daily.
351. The method according to one of items 256 to 313, wherein the therapeutically effective dose is about 250 mg of (2S)-2-[4-bromo-2-(1,2-oxazol-3-yl)phenoxy]propanoic acid and the composition is to be administered two times daily.
352. The method according to one of items 256 to 313, wherein the therapeutically effective dose is about 250 mg of (2S)-2-[4-bromo-2-(1,2-oxazol-3-yl)phenoxy]propanoic acid and the composition is to be administered three times daily.
353. The method according to one of items 256 to 313, wherein the therapeutically effective dose is about 250 mg of (2S)-2-[4-bromo-2-(1,2-oxazol-3-yl)phenoxy]propanoic acid and the composition is to be administered four times daily.
354. The method according to one of items 256 to 313, wherein the therapeutically effective dose is about 300 mg of (2S)-2-[4-bromo-2-(1,2-oxazol-3-yl)phenoxy]propanoic acid and the composition is to be administered one time daily.
355. The method according to one of items 256 to 313, wherein the therapeutically effective dose is about 300 mg of (2S)-2-[4-bromo-2-(1,2-oxazol-3-yl)phenoxy]propanoic acid and the composition is to be administered two times daily.
356. The method according to one of items 256 to 313, wherein the therapeutically effective dose is about 300 mg of (2S)-2-[4-bromo-2-(1,2-oxazol-3-yl)phenoxy]propanoic acid and the composition is to be administered three times daily.
357. The method according to one of items 256 to 313, wherein the therapeutically effective dose is about 300 mg of (2S)-2-[4-bromo-2-(1,2-oxazol-3-yl)phenoxy]propanoic acid and the composition is to be administered four times daily.
358. The method according to one of items 256 to 313, wherein the therapeutically effective dose is about 350 mg of (2S)-2-[4-bromo-2-(1,2-oxazol-3-yl)phenoxy]propanoic acid and the composition is to be administered one time daily.
359. The method according to one of items 256 to 313, wherein the therapeutically effective dose is about 350 mg of (2S)-2-[4-bromo-2-(1,2-oxazol-3-yl)phenoxy]propanoic acid and the composition is to be administered two times daily.
360. The method according to one of items 256 to 313, wherein the therapeutically effective dose is about 350 mg of (2S)-2-[4-bromo-2-(1,2-oxazol-3-yl)phenoxy]propanoic acid and the composition is to be administered three times daily.
361. The method according to one of items 256 to 313, wherein the therapeutically effective dose is about 350 mg of (2S)-2-[4-bromo-2-(1,2-oxazol-3-yl)phenoxy]propanoic acid and the composition is to be administered four times daily.
362. The method according to one of items 256 to 313, wherein the therapeutically effective dose is about 400 mg of (2S)-2-[4-bromo-2-(1,2-oxazol-3-yl)phenoxy]propanoic acid and the composition is to be administered one time daily.
363. The method according to one of items 256 to 313, wherein the therapeutically effective dose is about 400 mg of (2S)-2-[4-bromo-2-(1,2-oxazol-3-yl)phenoxy]propanoic acid and the composition is to be administered two times daily.
364. The method according to one of items 256 to 313, wherein the therapeutically effective dose is about 400 mg of (2S)-2-[4-bromo-2-(1,2-oxazol-3-yl)phenoxy]propanoic acid and the composition is to be administered three times daily.
365. The method according to one of items 256 to 313, wherein the therapeutically effective dose is about 400 mg of (2S)-2-[4-bromo-2-(1,2-oxazol-3-yl)phenoxy]propanoic acid and the composition is to be administered four times daily.
366. The method according to one of items 256 to 313, wherein the therapeutically effective dose is about 500 mg of (2S)-2-[4-bromo-2-(1,2-oxazol-3-yl)phenoxy]propanoic acid and the composition is to be administered two times daily.
367. The method according to one of items 256 to 313, wherein the therapeutically effective dose is about 500 mg of (2S)-2-[4-bromo-2-(1,2-oxazol-3-yl)phenoxy]propanoic acid and the composition is to be administered three times daily.
368. The method according to one of items 256 to 313, wherein the therapeutically effective dose is about 600 mg of (2S)-2-[4-bromo-2-(1,2-oxazol-3-yl)phenoxy]propanoic acid and the composition is to be administered two times daily.
369. The method according to one of items 256 to 313, wherein the therapeutically effective dose is about 600 mg of (2S)-2-[4-bromo-2-(1,2-oxazol-3-yl)phenoxy]propanoic acid and the composition is to be administered three times daily.
370. The method according to one of items 256 to 369, wherein the therapeutically effective dose is administered orally to the subject.
371. The method according to one of items 256 to 369, wherein the method further comprises administration of one or more additional active agents.
372. The method according to item 371, wherein the additional active agent is known to treat, prevent and/or ameliorate neuromuscular disorders.
373. The method according to any one of items 371 or 372, wherein the additional active agent enhances correct splicing of SMN2 and/or enhances SMN2 splicing.
374. The method according to any one of items 371 or 372, wherein the additional active agent is an antisense oligonucleotide (ASO) to enhance correct splicing of SMN2, such as nusinersen.
375. The method according to any one of items 371 or 372, wherein the additional active agent is gene therapy, such as onasemnogene abeparvovec-xioi, to provide an alternative source the SMN1 gene leading to increased SMN protein production.
376. The method according to any one of items 371 or 372, wherein the additional active agent is a small molecule to enhance SMN2 splicing, such as risdiplam.
377. The method according to item 371, wherein the additional active is selected from the group consisting of risdiplam, nusinersen, BIIB115, Salbutamol, GYM329, SRK-015, branaplam, tirasemtiv, reldesemtiv, talditercept alfa, taldefgrobep alfa, pyridostigmine, RG-6237, Apitegromab, amifampridine, nipocalimab, EXG-001307, and ACTX-401.
378. The method according to any one of items 371 to 377, wherein (2S)-2-[4-bromo-2-(1,2-oxazol-3-yl)phenoxy]propanoic acid, or a pharmaceutically acceptable salt, hydrate, polymorph, tautomer, or solvate thereof, and the additional active agent are administered simultaneously to the subject.
379. The method according to any one of items 371 to 377, wherein (2S)-2-[4-bromo-2-(1,2-oxazol-3-yl)phenoxy]propanoic acid, or a pharmaceutically acceptable salt, hydrate, polymorph, tautomer, or solvate thereof, and the additional active agent are administered at different times to the subject.
380. The method according to any one of items 371 to 377, wherein (2S)-2-[4-bromo-2-(1,2-oxazol-3-yl)phenoxy]propanoic acid, or a pharmaceutically acceptable salt, hydrate, polymorph, tautomer, or solvate thereof, and the additional active agent are administered sequentially.
381. A method for treatment of spinal muscular atrophy comprising administering (2S)-2-[4-bromo-2-(1,2-oxazol-3-yl)phenoxy]propanoic acid, or a pharmaceutically acceptable salt, hydrate, polymorph, tautomer, or solvate thereof, and an antisense oligonucleotide (ASO) to enhance correct splicing of SMN2 to a subject in need thereof.
382. The method according to item 381, wherein the antisense oligonucleotide (ASO) to enhance correct splicing of SMN2 is nusinersen or BIIB115.
383. A method for treatment of spinal muscular atrophy comprising administering (2S)-2-[4-bromo-2-(1,2-oxazol-3-yl)phenoxy]propanoic acid, or a pharmaceutically acceptable salt, hydrate, polymorph, tautomer, or solvate thereof, and a compound to enhance SMN2 splicing to a subject in need thereof.
384. The method according to item 383, wherein the compound to enhance SMN2 splicing is risdiplam.
385. The composition for use or the method according to any one of the preceding items, wherein the method further comprise administering a second therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject either one day, two days, three days, four days, five days, six days or at least seven days after a first therapeutically effective dose is administered.
386. The composition for use or the method according to item 385, wherein the second therapeutically effective dose is from 100 mg to about 1500 mg.
387. The composition for use or the method according to item 385, wherein the second therapeutically effective dose is the same as the first therapeutically effective dose administered to the subject.
388. The composition for use or the method according to any one of items 385 to 387, wherein the method further comprise administering a third therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject either one day, two days, three days, four days, five days, six days or at least seven days after the therapeutically effective dose is administered.
389. The composition for use or the method according to item 388, wherein the third therapeutically effective dose is from 100 mg to about 1500 mg.
390. The composition for use or the method according to item 388, wherein the third therapeutically effective dose is the same as the first therapeutically effective dose and/or the second therapeutically effective dose administered to the subject.
391. The composition for use or the method according to any one of items 1 to 384, wherein the administration of the therapeutically effective doses of (2S)-2-[4-bromo-2-(1,2-oxazol-3-yl)phenoxy]propanoic acid, or a pharmaceutically acceptable salt, hydrate, polymorph, tautomer, or solvate thereof, are repeated at least 1, 2, 3, 4, 5 or 6 times weekly.
392. The composition for use or the method according to any one of items 1 to 384, wherein the administration of the therapeutically effective doses of (2S)-2-[4-bromo-2-(1,2-oxazol-3-yl)phenoxy]propanoic acid, or a pharmaceutically acceptable salt, hydrate, polymorph, tautomer, or solvate thereof, are repeated at least 1-3 times weekly, 2-5 times weekly or 3-6 times weekly.
393. The composition for use or the method according to any one of items 1 to 384, wherein the administration of the therapeutically effective doses of (2S)-2-[4-bromo-2-(1,2-oxazol-3-yl)phenoxy]propanoic acid, or a pharmaceutically acceptable salt, hydrate, polymorph, tautomer, or solvate thereof, are repeated daily.
394. The composition for use or the method according to any one of items 1 to 384, wherein the administration of the therapeutically effective doses of (2S)-2-[4-bromo-2-(1,2-oxazol-3-yl)phenoxy]propanoic acid, or a pharmaceutically acceptable salt, hydrate, polymorph, tautomer, or solvate thereof, is repeated 1, 2, 3, 4, 5, 6, 7 or 8 times daily.
395. The composition for use or the method according to any one of items 1 to 384, wherein the administration of the therapeutically effective doses of (2S)-2-[4-bromo-2-(1,2-oxazol-3-yl)phenoxy]propanoic acid, or a pharmaceutically acceptable salt, hydrate, polymorph, tautomer, or solvate thereof, is repeated 1 to 8 times daily or 2 to 5 times daily.
396. The composition for use or the method according to any one of items 1 to 384, wherein the therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, is administered at least one time daily.
397. The composition for use or the method according to any one of items 1 to 384, wherein the therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, is administered one time daily.
398. The composition for use or the method according to any one of items 1 to 384, wherein the therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, is administered two times daily, three times daily, or four times daily.
399. The composition for use or the method according to any one of the preceding items, wherein the therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, is the daily dosage amount of (2S)-2-[4-bromo-2-(1,2-oxazol-3-yl)phenoxy]propanoic acid, or a pharmaceutically acceptable salt, hydrate, polymorph, tautomer, or solvate thereof.
400. The composition for use or the method according to item 399, wherein the daily dosage amount of (2S)-2-[4-bromo-2-(1,2-oxazol-3-yl)phenoxy]propanoic acid, or a pharmaceutically acceptable salt, hydrate, polymorph, tautomer, or solvate thereof is administered as a single dosage.
401. The composition for use or the method according to item 399, wherein the daily dosage amount of (2S)-2-[4-bromo-2-(1,2-oxazol-3-yl)phenoxy]propanoic acid, or a pharmaceutically acceptable salt, hydrate, polymorph, tautomer, or solvate thereof is administered in smaller dosages throughout the day.
402. The composition for use or the method according to item 399, wherein the daily dosage amount of (2S)-2-[4-bromo-2-(1,2-oxazol-3-yl)phenoxy]propanoic acid, or a pharmaceutically acceptable salt, hydrate, polymorph, tautomer, or solvate thereof is administered once a day or at least one time daily, such as twice a day, at least at two different time points throughout the day, three times a day or at least at three different time points throughout the day.
403. Use of a composition comprising (2S)-2-[4-bromo-2-(1,2-oxazol-3-yl)phenoxy]propanoic acid, or a pharmaceutically acceptable salt, hydrate, polymorph, tautomer, or solvate thereof, in the manufacture of a medicament for treatment of spinal muscular atrophy in a subject, wherein the composition is for administration at a therapeutic dose of 100 to 1500 mg of (2S)-2-[4-bromo-2-(1,2-oxazol-3-yl)phenoxy]propanoic acid.
404. A composition comprising (2S)-2-[4-bromo-2-(1,2-oxazol-3-yl)phenoxy]propanoic acid, or a pharmaceutically acceptable salt, hydrate, polymorph, tautomer, or solvate thereof, wherein the composition comprises 50 to 400 mg (2S)-2-[4-bromo-2-(1,2-oxazol-3-yl)phenoxy]propanoic acid.
405. The composition according to item 404, wherein the composition comprises 50 mg (2S)-2-[4-bromo-2-(1,2-oxazol-3-yl)phenoxy]propanoic acid, or a pharmaceutically acceptable salt, hydrate, polymorph, tautomer, or solvate thereof.
406. The composition according to item 404, wherein the composition comprises 100 mg (2S)-2-[4-bromo-2-(1,2-oxazol-3-yl)phenoxy]propanoic acid, or a pharmaceutically acceptable salt, hydrate, polymorph, tautomer, or solvate thereof.
407. The composition according to item 404, wherein the composition comprises 150 mg (2S)-2-[4-bromo-2-(1,2-oxazol-3-yl)phenoxy]propanoic acid, or a pharmaceutically acceptable salt, hydrate, polymorph, tautomer, or solvate thereof.
408. The composition according to item 404, wherein the composition comprises 200 mg (2S)-2-[4-bromo-2-(1,2-oxazol-3-yl)phenoxy]propanoic acid, or a pharmaceutically acceptable salt, hydrate, polymorph, tautomer, or solvate thereof.
409. The composition according to item 404, wherein the composition comprises 250 mg (2S)-2-[4-bromo-2-(1,2-oxazol-3-yl)phenoxy]propanoic acid, or a pharmaceutically acceptable salt, hydrate, polymorph, tautomer, or solvate thereof.
410. The composition according to item 404, wherein the composition comprises 300 mg (2S)-2-[4-bromo-2-(1,2-oxazol-3-yl)phenoxy]propanoic acid, or a pharmaceutically acceptable salt, hydrate, polymorph, tautomer, or solvate thereof.
411. The composition according to item 404, wherein the composition comprises 350 mg (2S)-2-[4-bromo-2-(1,2-oxazol-3-yl)phenoxy]propanoic acid, or a pharmaceutically acceptable salt, hydrate, polymorph, tautomer, or solvate thereof.
412. The composition according to item 404, wherein the composition comprises 400 mg (2S)-2-[4-bromo-2-(1,2-oxazol-3-yl)phenoxy]propanoic acid, or a pharmaceutically acceptable salt, hydrate, polymorph, tautomer, or solvate thereof.
413. The composition according to any one of items 404 to 412, wherein the composition further comprises at least one pharmaceutically acceptable adjuvant and/or excipient.
414. The composition according to any of items 404 to 413, wherein the composition is for oral administration.
415. The composition according to any one of items 404 to 414, wherein the composition is a solid dosage form.
416. The composition according to item 415, wherein the solid dosage form is selected from the group consisting of capsule, such as sprinkle capsule and gelatine capsule; tablet, such as uncoated tablet, coated tablet and slow-release tablet; and sprinkle.
417. The composition according to any one of items 415 to 416, wherein the solid dosage form comprises 50 mg to 400 mg of (2S)-2-[4-bromo-2-(1,2-oxazol-3-yl)phenoxy]propanoic acid or a pharmaceutically acceptable salt, hydrate, polymorph, tautomer, or solvate thereof.
418. The composition according to any one of items 415 to 416, wherein the solid dosage form comprises 100 mg of (2S)-2-[4-bromo-2-(1,2-oxazol-3-yl)phenoxy]propanoic acid or a pharmaceutically acceptable salt, hydrate, polymorph, tautomer, or solvate thereof.
419. The composition according to any one of items 415 to 416, wherein the solid dosage form comprises 150 mg of (2S)-2-[4-bromo-2-(1,2-oxazol-3-yl)phenoxy]propanoic acid or a pharmaceutically acceptable salt, hydrate, polymorph, tautomer, or solvate thereof.
420. The composition according to any one of items 415 to 416, wherein the solid dosage form comprises 200 mg of (2S)-2-[4-bromo-2-(1,2-oxazol-3-yl)phenoxy]propanoic acid or a pharmaceutically acceptable salt, hydrate, polymorph, tautomer, or solvate thereof.
421. The composition according to any one of items 415 to 416, wherein the solid dosage form comprises 250 mg of (2S)-2-[4-bromo-2-(1,2-oxazol-3-yl)phenoxy]propanoic acid or a pharmaceutically acceptable salt, hydrate, polymorph, tautomer, or solvate thereof.
422. The composition according to any one of items 415 to 416, wherein the solid dosage form comprises 300 mg of (2S)-2-[4-bromo-2-(1,2-oxazol-3-yl)phenoxy]propanoic acid or a pharmaceutically acceptable salt, hydrate, polymorph, tautomer, or solvate thereof.
423. The composition according to any one of items 415 to 416, wherein the solid dosage form comprises 350 mg of (2S)-2-[4-bromo-2-(1,2-oxazol-3-yl)phenoxy]propanoic acid or a pharmaceutically acceptable salt, hydrate, polymorph, tautomer, or solvate thereof.
424. The composition according to any one of items 415 to 416, wherein the solid dosage form comprises 400 mg of (2S)-2-[4-bromo-2-(1,2-oxazol-3-yl)phenoxy]propanoic acid or a pharmaceutically acceptable salt, hydrate, polymorph, tautomer, or solvate thereof.
425. The composition according to any of items 413 to 423, wherein the pharmaceutically acceptable adjuvant and/or excipient is selected from the group consisting of filler, binder, lubricant and disintegrant.
426. The composition according to any of items 413 to 423, wherein the pharmaceutically acceptable adjuvant and/or excipient is selected from the group consisting of silicified microcrystalline cellulose, microcrystalline cellulose, maltodextrin, magnesium stearate and croscarmellose sodium.
427. The composition according to any one of items 415 to 426, wherein the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.
428. The composition according to any one of items 404 to 414, wherein the composition is in the form of a liquid, liquid suspension, oil, emulsion, or syrup.
429. The composition according to any of items 404 to 428, wherein the composition comprises 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof.
430. The composition according to any of items 404 to 428, wherein the composition comprises 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof.
431. The composition according to any of items 404 to 428, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant; and
   e. 0.25 to 5 wt %, such as 0.3 to 2.5 wt % disintegrant;
   with the proviso that the sum of the wt % of the components does not exceed 100 wt %.
432. The composition according to any of items 404 to 428, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate; and
   f. 0.25 to 5 wt %, such as 0.3 to 2.5 wt % Croscarmellose sodium;
   with the proviso that the sum of the wt % of the components does not exceed 100 wt %.
433. The composition according to any of items 404 to 428, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant;
   e. 0.25 to 5 wt %, such as 0.3 to 2.5 wt % disintegrant; and
   f. 1 to 10 wt % film coating;
   with the proviso that the sum of the wt % of the components does not exceed 100 wt %.
434. The composition according to any of items 404 to 428, wherein the composition comprises or consists of:
   a. 10 to 80 wt %, such as 40 to 65 wt %, such as 50 to 60 wt %, such as 50 to 55 wt %, such as 55 to 60 wt %, such as about 53 wt %, such as 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate;
   f. 0.25 to 5 wt %, such as 0.3 to 2.5 wt % Croscarmellose sodium; and
   g. 1 to 10 wt % film coating composition such as Opadry white;
   with the proviso that the sum of the wt % of the components does not exceed 100 wt %.
435. The composition according to any of items 404 to 428, wherein the composition comprises or consists of:
   a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate;
   f. 0.25 to 5 wt %, such as 0.3 to 2.5 wt % Croscarmellose sodium; and
   g. 1 to 10 wt % film coating composition such as Opadry white,
   with the proviso that the sum of the wt % of the components does not exceed 100 wt %.
436. The composition according to any one of items 404 to 435 for use in the treatment of spinal muscular atrophy.
437. A method for treatment of spinal muscular atrophy comprising administering kit-of-parts according to any one of items 404 to 435 to a subject in need thereof.
438. Use of a kit-of-parts according to any one of items 404 to 435 for the manufacture of a medicament for the treatment of spinal muscular atrophy.
439. A kit-of-parts comprising:
   a. (2S)-2-[4-bromo-2-(1,2-oxazol-3-yl)phenoxy]propanoic acid, or a pharmaceutically acceptable salt, hydrate, polymorph, tautomer, or solvate thereof; and
   b. a compound that increases the amount of functional SMN protein produced by SMN2.
440. The kit-of-parts according to item 439, wherein the kit-of-parts comprises 100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof.
441. The kit-of-parts according to any one of items 439 or 440, wherein the compound that increases the amount of functional SMN protein produced by SMN2 is risdiplam.
442. The kit-of-parts according to any one of items 439 or 440, wherein the compound that increases the amount of functional SMN protein produced by SMN2 is nusinersen.
443. The kit-of-parts according to any one of items 439 or 440, wherein the compound that increases the amount of functional SMN protein produced by SMN2 is branaplam.
444. The kit-of-parts according to any one of items 439 or 440, wherein the compound that increases the amount of functional SMN protein produced by SMN2 is BIIB115.
445. The kit-of-parts according to any one of items 439 or 440, wherein the kit-of-parts comprises:
   a. 100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof; and
   b. 0.2 to 5 mg risdiplam, or a pharmaceutically acceptable salt, hydrate, polymorph, tautomer, or solvate thereof.
446. The kit-of-parts according to any one of items 439 or 440, wherein the kit-of-parts comprises:
   a. 100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof; and
   b. 12 to 60 mg nusinersen, or a pharmaceutically acceptable salt, hydrate, polymorph, tautomer, or solvate thereof.
447. The kit-of-parts according to any one of items 439 to 446, wherein the kit-of-parts further comprises an agent for increasing the Ca²⁺ sensitivity of the contractile filaments in muscle, such as tirasemtiv or reldesemtiv.
448. The kit-of-parts according to any one of items 439 to 447, wherein the kit-of-parts further comprises one or more additional compounds, such as salbutamol, GYM329, SRK-015, talditercept alfa, taldefgrobep alfa, pyridostigmine, RG-6237, Apitegromab, amifampridine, nipocalimab, EXG-001307, and/or ACTX-401.
449. The kit-of-parts according to any one of items 439 to 448 for use in the treatment of spinal muscular atrophy.
450. A method for treatment of spinal muscular atrophy comprising administering kit-of-parts according to any one of items 439 to 448 to a subject in need thereof.
451. Use of a kit-of-parts according to any one of items 439 to 448 for the manufacture of a medicament for the treatment of spinal muscular atrophy.
452. The composition for use, the method or the kit-of-parts for use according to any one of the preceding items, wherein the subject is a human being.
453. The composition for use, the method or the kit-of-parts for use according to any one of the preceding items, wherein the subject is suffering from symptoms of SMA.
454. The composition for use, the method or the kit-of-parts for use according to any one of the preceding items, wherein the subject has been diagnosed with SMA.
455. The composition for use, the method or the kit-of-parts for use according to any one of the preceding items, wherein the subject has a deletion or mutation in each survival motor neuron 1 (SMN1) allele.
456. The composition for use, the method or the kit-of-parts for use according to any one of the preceding items, wherein the subject is homozygous for a SMN1 gene mutation.
457. The composition for use, the method or the kit-of-parts for use according to any one of the preceding items, wherein the subject has been diagnosed with SMA type 0.
458. The composition for use, the method or the kit-of-parts for use according to any one of the preceding items, wherein the subject has been diagnosed with SMA type 0 and has undergone a gene therapy to provide an alternative source the SMN1 gene leading to increased SMN protein production.
459. The composition for use, the method or the kit-of-parts for use according to any one of the preceding items, wherein the subject has been diagnosed with SMA type 0, has undergone a gene therapy to provide an alternative source the SMN1 gene leading to increased SMN protein production and is treated with a therapy that increases the amount of functional SMN protein produced by SMN2.
460. The composition for use, the method or the kit-of-parts for use according to any one of the preceding items, wherein the subject has been diagnosed with SMA type 0 and is treated with a therapy that increases the amount of functional SMN protein produced by SMN2.
461. The composition for use, the method or the kit-of-parts for use according to any one of the preceding items, wherein the subject has been diagnosed with SMA type 1.
462. The composition for use, the method or the kit-of-parts for use according to any one of the preceding items, wherein the subject has been diagnosed with SMA type 1 and has undergone a gene therapy to provide an alternative source the SMN1 gene leading to increased SMN protein.
463. The composition for use, the method or the kit-of-parts for use according to any one of the preceding items, wherein the subject has been diagnosed with SMA type 1, has undergone a gene therapy to provide an alternative source the SMN1 gene leading to increased SMN protein production and is treated with a therapy that increases the amount of functional SMN protein produced by SMN2.
464. The composition for use, the method or the kit-of-parts for use according to any one of the preceding items, wherein the subject has been diagnosed with SMA type 1 and is treated with a therapy that increases the amount of functional SMN protein produced by SMN2.
465. The composition for use, the method or the kit-of-parts for use according to any one of the preceding items, wherein the subject has been diagnosed with SMA type 2.
466. The composition for use, the method or the kit-of-parts for use according to any one of the preceding items, wherein the subject has been diagnosed with SMA type 2 and has undergone a gene therapy to provide an alternative source the SMN1 gene leading to increased SMN protein production.
467. The composition for use, the method or the kit-of-parts for use according to any one of the preceding items, wherein the subject has been diagnosed with SMA type 2, has undergone a gene therapy to provide an alternative source the SMN1 gene leading to increased SMN protein production and is treated with a therapy that increases the amount of functional SMN protein produced by SMN2.
468. The composition for use, the method or the kit-of-parts for use according to any one of the preceding items, wherein the subject has been diagnosed with SMA type 2 and is treated with a therapy that increases the amount of functional SMN protein produced by SMN2.
469. The composition for use, the method or the kit-of-parts for use according to any one of the preceding items, wherein the subject has been diagnosed with SMA type 3.
470. The composition for use, the method or the kit-of-parts for use according to any one of the preceding items, wherein the subject has been diagnosed with SMA type 3 and has undergone a gene therapy to provide an alternative source the SMN1 gene leading to increased SMN protein production.
471. The composition for use, the method or the kit-of-parts for use according to any one of the preceding items, wherein the subject has been diagnosed with SMA type 3, has undergone a gene therapy to provide an alternative source the SMN1 gene leading to increased SMN protein production and is treated with a therapy that increases the amount of functional SMN protein produced by SMN2.
472. The composition for use, the method or the kit-of-parts for use according to any one of the preceding items, wherein the subject has been diagnosed with SMA type 3 and is treated with a therapy that increases the amount of functional SMN protein produced by SMN2.
473. The composition for use, the method or the kit-of-parts for use according to any one of the preceding items, wherein the subject has been diagnosed with SMA Type 4.
474. The composition for use, the method or the kit-of-parts for use according to any one of the preceding items, wherein the subject has been diagnosed with SMA Type 4 and is treated with a therapy that increases the amount of functional SMN protein produced by SMN2.
475. The composition for use, the method or the kit-of-parts for use according to any one of the preceding items, the gene therapy is onasemnogene abeparvovec-xioi.
476. The composition for use, the method or the kit-of-parts for use according to any one of the preceding items, wherein the therapy that increases the amount of functional SMN protein produced by SMN2 is nusinersen and/or risdiplam.
477. The composition for use, the method or the kit-of-parts for use according to any one of the preceding items, wherein the subject does not have hyperuricemia.
478. The composition for use, the method or the kit-of-parts for use according to any one of the preceding items, wherein the subject has a level of serum uric acid below 6.5 mg/dL.

### EXAMPLES

### Example 1: SMA Model

### Brief Description of the Study

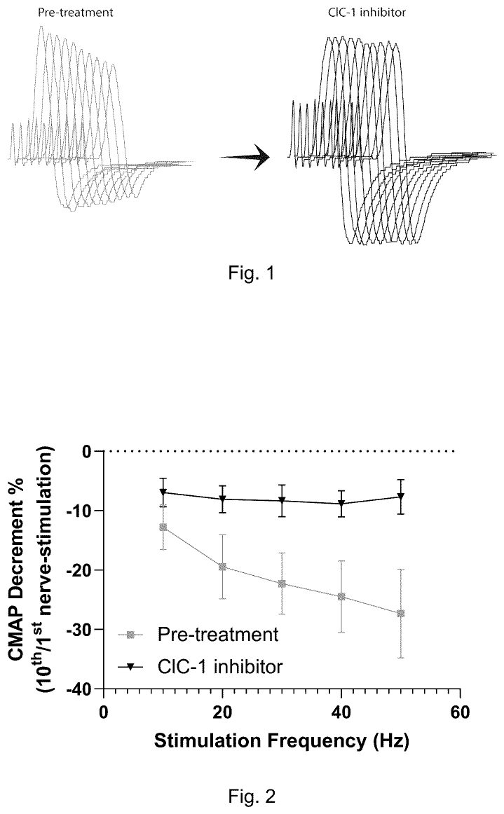

The employed model of SMA is a genetic knock-in model of severe SMA (“delta-7 mouse”). This model was developed in The Ohio State University laboratories (Foust et al, 2010). In this study the mice were administered at post-natal day 2 via intracerebroventricular injection 40 μg of morpholino ASOs directed against ISS-N1 to increase full-length SMN protein production from SMN2 to allow the animal to live to about 90 days. Five animals aged 67 days were investigated in a study where 20 mg/kg CIC-1 inhibitor ((S)-2-(4-bromo-2-ethynylphenoxy)propionic acid) in phosphate buffered saline was administered intraperitoneally and subjected to a rotarod test battery 90 minutes after injection (single intervention) and CMAP recording 120 min after injection. The animals did not exhibit any adverse reactions to the compound injection, nor any signs of muscle stiffness. All animals performed better in the running test and displayed reduced decrement in EMG (RNS) at all tested frequencies, see FIGS. 1, 2 and 3.

### Methods

All studies were approved by the animal institutional care and use committee of The Ohio State University (OH, USA).

### Rotarod

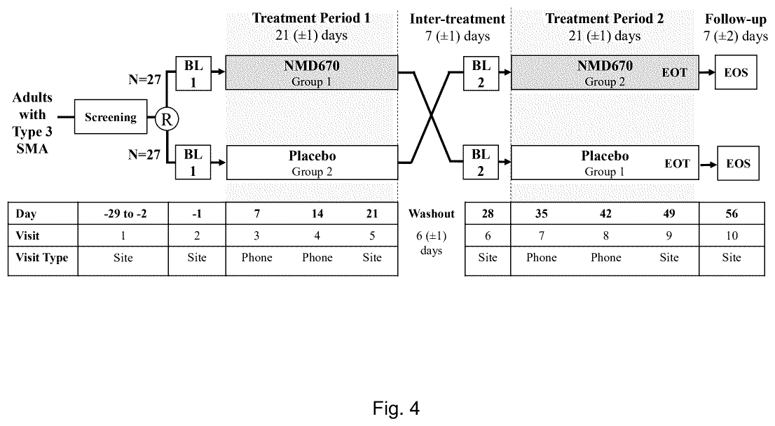

RotaRod (Harward apparatus, MA, USA) were used to explore the physical capabilities of the SMA mouse model in vivo and examined coordination ability of legs and endurance on a horizontal accelerating, rotating rod. The RotaRod test was designed to start at a low revolution rate of 5 rpm of the horizontal roll and accelerate gradually to 45 rpm over 300 seconds (maximal test time). Animals were acclimatized and trained for several weeks prior to intervention, to avoid training bias. Animals were noted for how long they were able to stay on the acceleration rotating rod, and the maximal time was noted as the “latency to fall”. This was performed prior to dosing, and then 90 min after IP dosing of 20 mg/kg of the CIC-1inhibitor. The results are shown in FIG. 3 and Table 1. All animals were able to stay on the rotating rod longer after treatment.

TABLE 1
Time for remaining on rotor rod (s)

| Pre-treatment | CIC-1 inhibitor |
|---|---|
| 62.2 ± 40.2 | 81 ± 63.2 |

### Electromyography (EMG)

Compound Muscle Action Potentials (CMAP) measurements were performed as previously described (Arnold et al, 2015). Briefly, the right sciatic nerve was stimulated with a pair of insulated 28-gauge monopolar needles (Teca, Oxford Instruments Medical, NY) placed in proximity to the sciatic nerve in the proximal hind limb. Recording electrodes consisted of a pair of fine ring wire electrodes (Alpine Biomed, Skovlunde, Denmark). The active recording electrode was placed distal to the knee joint over the proximal portion of the triceps surae muscle and the reference electrode over the metatarsal region of the foot. A disposable strip electrode (Carefusion, Middleton, WI) was placed on the contralateral hind limb to serve as the ground electrode. Data collection and analysis was performed using Cadwell Sierra Summit EMG unit (Cadwell laboratories, Kennewick, Washington, USA). Animals were stimulated in trains of 10 pulses of 50 μs at supramaximal voltage at 10, 20, 30, 40 and 50 Hz, with 10 seconds between stimulation trains.

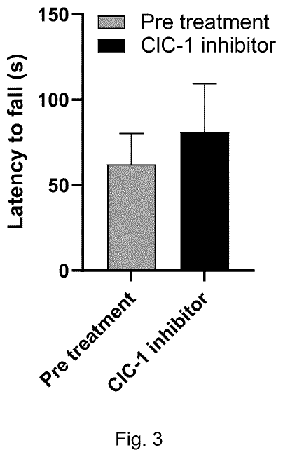

This was performed prior to dosing, and then 120 min after IP dosing of 20 mg/kg of the CIC-1inhibitor. The results are shown in FIG. 2 and Table 2. The relative amplitude from the 10th to the 1st stimulation is denoted as decrement, and a larger negative number designates a larger deficit/decrement, indicating a larger transmission failure from nerve to muscle. The decrement was less pronounced in all animals at all frequencies, but was only statistically significantly different between pre-treated and treated animals from 20 to 50 Hz.

TABLE 2
Decrement in CMAP measurements

| | |
|---|---|
| Stimulation | CMAP Decrement % |
| Frequency | (10th/1st Stimulation) |

| (Hz) | Pre-treatment | CIC-1 inhibitor |
|---|---|---|
| 10 | −12.8 ± 8.4 | −7.0 ± 5.4 |
| 20 | −19.4 ± 12.1 | −8.1 ± 5.1 |
| 30 | −22.3 ± 11.6 | −8.4 ± 6.0 |
| 40 | −24.5 ± 13.6 | −8.9 ± 4.9 |
| 50 | −27.3 ± 16.7 | −7.7 ± 6.5 |

### Example 2: Tablet Formulation

The immediate release tablets were formulated using standard excipients as described in table 3.

A standard high shear granulation process was developed. Microcrystalline cellulose was used as filler and binder excipient and maltodextrin was also applied as binder. The granulate contained 50-90% drug substance.

The granulate was dried and sieved and mixed with excipients to form a free-flowing blend. Silicified microcrystalline cellulose was added as a filler, Croscarmellose Sodium as a disintegrant and magnesium stearate as a lubricant. Silicified microcrystalline cellulose was found superior to microcrystalline cellulose as filler to achieve low weight variation. A single punch Diaf tablet press was used to manufacture the core tablets. The core tablets were film-coated with a standard white film-coating pre-mix (Opadry 03F180011 white) consisting of Hypromellose, polyethylene glycol 8000 and titanium dioxide. The term “Opadry White” refers to a composition coating prepared using Opadry® White obtained from Colorcon Pa, USA, in the form of the product sold as product code 03F180011 in 2022.

TABLE 3
Tablet Formulation

| Substance | | | |
|---|---|---|---|
| Quantity Per | Quantity Per | Quantity Per | |
| Tablet (mg) | Tablet (mg) | Tablet (mg) | Function |
| Product Strength | | | |
| 50 mg | 100 mg | 300 mg | |

Active ingredient

| | | | | |
|---|---|---|---|---|
| (2S)-2-[4-bromo-2-(1,2-oxazol- | 50.0 | 100.0 | 300.0 | Drug |
| 3-yl)phenoxy]propanoic acid | | | | substance |

Excipients of tablet core

| | | | | |
|---|---|---|---|---|
| Silicified microcrystalline | 2.5-30 | 5-60 | 15-180 | Filler |
| cellulose | | | | |
| Microcrystalline cellulose | 1-30 | 2-60 | 6-180 | Binder |
| Maltodextrin | 0.5-7 | 1-14 | 3-42 | Binder |
| Magnesium Stearate | 0.12-1.5 | 0.25-3 | 0.75-9 | Lubricant |
| Croscarmellose Sodium | 0.12-2.5 | 0.25-5 | 0.75-15 | Disintegrant |

Excipient of the coating

| | | | | |
|---|---|---|---|---|
| Opadry 03F180011 white | 0.5-5 | 1-10 | 3-30 | Film-coating |
| | | | | pre-mix |
| Total | 94.5 | 189 | 567 | |

15-30 mg/30-60 mg/90-180 mg Purified water (Ph.Eur), respectively was used and evaporated during processing of the NMD670 50 mg tablet, NMD670 100 mg tablet and NMD670 300 mg tablet, respectively.

### Example 3: Tablet Dissolution

Tablet dissolution was determined as follows.

TABLE 4
Chromatographic conditions

| Apparatus | HPLC system with UV/DAD detector | | |
|---|---|---|---|
| Column | XBridge BEH phenyl 2.5 μm 3.0 × 150 mm | | |
| Column temperature | 40° C. | | |
| Wavelength | 220 nm UV (DAD) detector | | |
| Flow | 0.6 mL/minute | | |
| Injection volume | 3 μL | | |
| Auto sampler | Ambient | | |
| temperature | | | |
| Mobile phase A (MFA): | Milli Q water/Acetonitrile/TFA, 80/20/0.05% | | |
| Mobile phase B (MFB): | Milli Q water/Acetonitrile/TFA, 5/95/0.05% | | |
| Mobile phase | Gradient | | |
| Gradient | Time | % MFA | % MFB |
| | 0.00 | 90 | 10 |
| | 1.00 | 90 | 10 |
| | 11.00 | 10 | 90 |
| | 12.00 | 10 | 90 |
| | 12.10 | 90 | 10 |
| | 16.00 | 90 | 10 |

| | |
|---|---|
| Runtime | 16.00 min |

TABLE 5
Dissolution System

| Apparatus | USP2 (Paddles) | |
|---|---|---|
| Medium | Dissolution Media pH 6.8 | |
| Media Volume | 900 | ml |
| Paddle Speed | 75 | RPM |
| Temperature | 37° | C. |
| Sample Extraction | 0, 15, 30, 45, 60, 90, 120 | |
| Timepoints | minutes | |
| Filter | 10 | μm |
| Dissolution profile | No replacement of dissolution | |
| | media after withdrawal of sample | |
| Sampling Volume | 1.5 | mL |

| | |
|---|---|
| Number of Vessels | 6 |

Dissolution Media pH 6.8 was prepared by dissolving 27.3 g Na₂HPO₄·2H₂O and 4.9 g Citric Acid in 1 L Milli-Q water. The pH was measured and adjusted if necessary to pH 6.8.

900 mL of dissolution media pH 6.8 was accurately transferred into each dissolution vessel. The apparatus was assembled, and the dissolution media was heated to 37° C.±0.5° C. The T=0 point was extracted from each vessel prior to dropping the tablets into the dissolution vessels. One tablet was gently dropped into each dissolution vessel, and rotation of the paddle was immediately started. Samples were taken that the sample extraction timepoints and analysed using the HPLC method. Not less than 80% of NMD670 is released from the tablets after a maximum of 60 minutes when performing release testing of the tablets.

### Example 4: Bioanalysis of Human Plasma Samples

### Sample Collection Method

Collect blood (3 mL recommended) onto wet ice (K₂EDTA tubes)
Immediately cool blood on crushed wet ice for maximum of 30 minutes
Harvest plasma as quickly as possible using refrigerated centrifuge by centrifuging at 2000 g for 10 minutes at 4° C.
Keep plasma on wet ice and immediately stabilise with 1:1 (v/v) addition of Water: Orthophosphoric Acid (100:2 v/v) prepared using ≥85% orthophosphoric acid and deionised water (e.g. 750 μL plasma+750 μL stabiliser).
Mix sample thoroughly.
Keep the stabilised plasma on wet ice during subsequent steps.
Divide the stabilised plasma into 5×250 μL aliquots in plastic cryotubes.
Store aliquots at nominal −70° C./−80° C. promptly.

### Sample Preparation Procedure

1. Thaw frozen calibration standards, QC samples, blank matrix and study samples on wet ice.
2. Vortex-mix all samples thoroughly.
3. On wet ice aliquot 150 μL of calibration standards, QC samples, study samples, and blanks into a 2 mL 96-well plate. For the reagent blank add the same volume of Water.
4. On wet ice add 25 μL of Methanol:Water (50:50) to wells containing blanks and the same volume of ISTD intermediate solution to all other wells.
5. On wet ice add 125 μL of Water to each well. Cap plate and vortex-mix (~5 minutes, 1000 rpm).
6. Centrifuge plate (~3000 g, 3 minutes, room temperature).
7. Condition SPE plate (Waters Oasis HLB μElution) with 250 μL Methanol.
8. Condition SPE plate with 250 μL of Water.
9. Load 250 μL of the sample onto the SPE plate (aspirate from the rim of the cone to avoid any debris collected at the bottom of the well). Pass through using minimum pressure.
10. Wash SPE plate with 250 μL of Water.
11. Wash SPE plate with 250 μL of Methanol:Water (5:95). Briefly increase pressure for one minute to ensure sorbent is partially dried.
12. Elute from SPE plate into a 1.2 mL 96-well plate using 30 μL of Methanol. Stand for one minute then pass through using absolute minimum pressure.
13. Elute again with another 30 μL of Methanol, collecting into the same plate. Briefly increase pressure for one minute to complete the elution.
14. Add 125 μL of Ammonium Formate 10 mM (aq):Formic Acid (100:0.3) to each well. Cap plate and vortex mix (~1 minute, ≥1200 rpm).
15. Store plate refrigerated prior to analysis.

TABLE 6
HPLC Conditions

| HPLC instrumentation | Waters, Acquity Classic, UPLC | |
|---|---|---|
| | Auxiliary pump, Switching valve | |
| Analytical column | Astec CHIROBIOTIC T 250 × 4.6 mm, 5 μm | |
| | (Supelco catalogue number 12024AST) | |
| In-line filter | KrudKatcher ULTRA HPLC In-Line Filter | |
| | 2 μm, Phenomenex (p/n AF0-8497) | |
| Column temperature | 25° | C. |
| setting | | |
| Autosampler temperature | 5° | C. |
| Mobile Phase A | Ammonium Formate 10 mM (aq):Formic | |
| | Acid (100:0.3) | |
| Mobile Phase B | Methanol | |
| Back pressure (typical) | 3000 | psi |
| Injection volume | 6-15 | μL |

TABLE 7
Gradient Conditions

| Time (minutes) | A (%) | B (%) | Flow rate (mL/min) |
|---|---|---|---|
| Initial | 67.5 | 32.5 | 1.05 |
| 8.00 | 27.5 | 72.5 | 1.05 |
| 8.05 | 1.0 | 99.0 | 1.05 |
| 8.30 | 1.0 | 99.0 | 1.05 |
| 9.50 | 1.0 | 99.0 | 2.00 |
| 10.50 | 1.0 | 99.0 | 2.00 |
| 10.55 | 67.5 | 32.5 | 2.00 |
| 12.50 | 67.5 | 32.5 | 1.05 |

TABLE 8
Mass Spectrometer Parameters

| Mass spectrometer | Sciex API 5000 | |
|---|---|---|
| Ionization | ESI- | |
| Resolution | Q1: Unit | |
| | Q3: Unit | |
| IonSpray voltage | −4500 V | |
| Temperature | 550° C. | |
| Collision gas (CAD) | Nitrogen | Setting: 9 |
| Curtain gas (CUR) | Nitrogen | Setting: 30 |
| Nebulizing gas (GS1) | Nitrogen | Setting: 55 |
| Auxiliary gas (GS2) | Nitrogen | Setting: 65 |

| | |
|---|---|
| Entrance potential (EP) | −10 V |
| Pause time | 5 ms |
| Acquisition time | Delay time: 210 seconds delay |
| | Acquisition time after delay: 4.80 |
| | minutes |
| | Total time: 8.30 minutes |
| Cycle time | 13.3 min (injection start to next |
| | injection start) |

TABLE 9
Compound Detection Parameters

| Compound | Transition | Dwell | DP | CE | CXP | Approximate RT |
|---|---|---|---|---|---|---|
| Name | Monitored | Time (ms) | (V) | (V) | (V) | and window (min) |
| NMD670 | 310.0 → 237.9 | 60 | −80 | −19 | −12 | 5.80 min (4.06-7.54) |
| ISTD1* | 315.0 → 242.9 | 60 | −80 | −19 | −12 | |

*ITSD1 is NMD670-d₃.

### Example 5: Phase I Clinical Trial

Part A1 tested single doses of NMD670 in a double-blind, randomised, placebo-controlled, partial crossover and dose-escalating design in healthy male subjects. A total of nine dose levels were investigated in three cohorts of subjects. Each cohort consisted of nine subjects, each subject had three study sessions. Each subject received escalating doses of NMD670 on two occasions and placebo on one occasion, the order will be randomized in a cross-over fashion. Each dose level was randomized in a 6:3 ratio (active vs. placebo). See Table 10 for an overview of the randomization scheme.

TABLE 10
Randomisation Scheme

| Sequence | Period 1 | Period 2 | Period 3 |
|---|---|---|---|
| 1 | Placebo | Dose 2 | Dose 3 |
| 2 | Dose 1 | Placebo | Dose 3 |
| 3 | Dose 1 | Dose 2 | Placebo |

Dose escalation was stopped after an adverse event of myotonia of moderate intensity observed in one subject administered 1600 mg of NMD670 at dose level 7 (spontaneously and fully resolved within hours). Due to this temporary halt and partial unblinding of the study during dose level 7, a new randomization was necessary. After unblinding three subjects in dose level 7, the original randomization for dose 8 and 9 was changed in order to keep the study blinded. As subjects of cohort 3 only had 2 occasions left, the randomisation of the original design (3-way cross-over) was not possible without compromising the ratio of active and placebo treated subjects, and therefore the within-subject comparison which is important for the evaluation of PD markers. Therefore, for the remaining 2 doses, a full cross-over design investigating one previously tested dose level was used. The 9 subjects in cohort 3 were randomized to receive the study drug in level 8 and placebo in level 9, or vice versa. To determine the effect of food on the exposure of single oral doses of NMD670, dose level 5 was administered in both the fasted and fed condition. Subjects who received dose level 5 returned for a fourth visit in which they received dose level 5 (or matching placebo) in the fed condition, in the same randomization as the chosen dose level in the fasted condition.

Part A2 of the study investigated the safety, tolerability and pharmacokinetics of NMD670 in 8 healthy female subjects of non-childbearing potential, in a randomized, double-blind, placebo-controlled single dose administration of NMD670. Subjects received 800 mg NMD670. Subjects were randomized in a 6:2 ratio (active vs. placebo).

### Main Inclusion Criteria Healthy Volunteers (Part A)

1. Signed informed consent prior to any study-mandated procedure
2. Part A1: Healthy male subjects, 18 to 45 years of age, inclusive at screening.
3. Part A2: Healthy female subjects of non-childbearing potential, 18-65 years of age, inclusive at screening.
5. Body mass index (BMI) between 18 and 30 kg/m2, inclusive at screening, and with a minimum weight of 50 kg.
6. All males must practice effective contraception during the study and be willing and able to continue contraception for at least 90 days after their last dose of study treatment.
7. Has the ability to communicate well with the Investigator in the Dutch language and willing to comply with the study restrictions.

### Main Exclusion Criteria Healthy Volunteers (Part A)

1. Evidence of any active or chronic disease or condition that could interfere with or for which the treatment of might interfere with, the conduct of the study, or that would pose an unacceptable risk to the subject in the opinion of the investigator (following a detailed medical history, physical examination, vital signs (systolic and diastolic blood pressure, pulse rate, body temperature) and 12-lead electrocardiogram (ECG). Minor deviations from the normal range may be accepted, if judged by the Investigator to have no clinical relevance.
2. Clinically significant abnormalities, as judged by the investigator, in laboratory test results (including hepatic and renal panels, complete blood count, chemistry panel and urinalysis). In the case of uncertain or questionable results, tests performed during screening may be repeated before randomization to confirm eligibility or judged to be clinically irrelevant for healthy subjects.
3. Positive Hepatitis B surface antigen (HBsAg), Hepatitis C antibody (HCV Ab), or human immunodeficiency virus antibody (HIV Ab) at screening.
4. Systolic blood pressure (SBP) greater than 140 or less than 90 mm Hg, and diastolic blood pressure (DBP) greater than 90 or less than 50 mm Hg at screening.
5. Abnormal findings in the resting ECG at screening defined as:
   a. QTcF>450 or <300 msec for men and QTcF>470 or <300 msec for women
   b. Notable resting bradycardia (HR<45 bpm) or tachycardia (HR>100 bpm)
   c. Personal or family history of congenital long QT syndrome or sudden death;
   d. ECG with QRS and/or T wave judged to be unfavourable for a consistently accurate QT measurement (e.g., neuromuscular artefact that cannot be readily eliminated, arrhythmias, indistinct QRS onset, low amplitude T wave, merged T- and U-waves, prominent U waves);
   e. Evidence of atrial fibrillation, atrial flutter, complete branch block, Wolf-Parkinson-White Syndrome, or cardiac pacemaker
6. Use of any medications (prescription or over-the-counter [OTC]), within 14 days of study drug administration, or less than 5 half-lives (whichever is longer). Exceptions are paracetamol (up to 4 g/day) and ibuprofen (up to 1 g/day). Other exceptions will only be made if the rationale is clearly documented by the investigator.
7. Use of any vitamin, mineral, herbal, and dietary supplements within 7 days of study drug administration, or less than 5 half-lives (whichever is longer). Exceptions will only be made if the rationale is clearly documented by the investigator.
8. Participation in an investigational drug or device study (last dosing of previous study was within 90 days prior to first dosing of this study).
9. History of abuse of addictive substances (alcohol, illegal substances) or current use of more than 21 units alcohol per week, drug abuse, or regular user of sedatives, hypnotics, tranquillisers, or any other addictive agent.
10. Positive test for drugs of abuse at screening or pre-dose. Retesting is allowed at the discretion of the Investigator.
11. Alcohol will not be allowed from at least 24 hours before screening or pre-dose.
12. Smoker of more than 10 cigarettes per day prior to screening or who use tobacco products equivalent to more than 10 cigarettes per day and unable to abstain from smoking whilst in the unit.
13. Subjects will not be allowed to have excessive caffeine consumption, defined as >800 mg per day.
14. Any confirmed significant allergic reactions (urticaria or anaphylaxis) against any drug, or multiple drug allergies (non-active hay fever is acceptable).
15. Loss or donation of blood over 500 mL within three months (males) or four months (females) prior to screening or intention to donate blood or blood products during the study.
16. Any known factor, condition, or disease that might interfere with treatment compliance, study conduct or interpretation of the results such as drug or alcohol dependence or psychiatric disease.
17. History of trauma to the lower extremities or other conditions (most importantly neurological or muscle diseases) that, in the opinion of the investigator, could affect the electrophysiological measurements.
18. Excessive exercise within 7 days before study drug administration.
19. Clinically significant abnormalities in coagulation.

### Concomitant Medications for Healthy Volunteers (Part A)

No prescription medications and OTC medications were permitted within 14 days prior to study drug administrations, or less than 5 half-lives (whichever is longer), and during the course of the study.

No vitamin, mineral, herbal, and dietary supplements will be permitted within 7 days prior to study drug administrations, or less than 5 half-lives (whichever is longer), and during the course of the study.

Exceptions were paracetamol (up to 4 g/day) and ibuprofen (up to 1 g/day). Other exceptions were only be made if the rationale was clearly documented by the investigator. No exceptions were made for drugs that could influence the MVRC outcomes, such as sodium channel blockers, dantrolene, or anti-epileptic drugs. Use of substrates of CYP2C9, and CYP2C19 were prohibited. For CYP2C19 substrates, a) substrates exhibiting ≥5-fold increase in substrate AUC with CYP2C19 inhibitors: s-mephenytoin, omeprazole; or b) substrates exhibiting ≥2-fold but <5-fold increase in substrate AUC: diazepam, lansoprazole, rabeprazole, voriconazole were prohibited. For CYP2C9 substrates, a) substrates exhibiting ≥5-fold increase in substrate AUC with CYP2C9 Inhibitors: celecoxib; or b) substrates exhibiting ≥2-fold but <5-fold increase in substrate AUC: glimepiride, phenytoin, tolbutamide, warfarin were prohibited.

Use of any vaccine (initial or follow-up): there was a minimum period of 1 week between vaccination and screening; and 1 week between vaccination and dosing; until end of study.

### Tolerability/Safety Endpoints

The following endpoints were determined at time points indicated in the Schedule of Assessments.
Serious adverse events (SAEs) and adverse events (AEs) were collected throughout the study at every study visit.
Concomitant medication
Clinical laboratory tests
   Haematology
   Chemistry
   Urinalysis
   Coagulation
Vital signs
   Pulse Rate (bpm)
   Systolic blood pressure (mmHg)
   Diastolic blood pressure (mmHg)
   Respiratory rate
ECG
   Heart Rate (HR) (bpm), PR, QRS, QT, QTcF
24-hour Holter recording
Hand grip dynamometry
   Grip release profile; time profile from 100% maximum voluntary contraction (MVC) to 5% of the 100% MVC

### Results of Phase 1 (Part A)

The demographic characteristics of the 35 subjects enrolled in Part A of the Phase 1 study are presented in Table 11.

TABLE 11
Demographic Characteristics of 35 Subjects

| | | | |
|---|---|---|---|
| Age | Mean (SD) | 34.7 | (14.0) |
| Weight (kg) | Mean (SD) | 76.2 | (10.2) |
| Height (cm) | Mean (SD) | 178.5 | (9.8) |
| BMI | Mean (SD) | 24.0 | (3.1) |

| | | |
|---|---|---|
| Sex | Female (n) | 8 |
| | Male (n) | 27 |
| Race | Asian (n) | 3 |
| | Black (n) | 1 |
| | Mixed (n) | 2 |
| | Other (n) | 1 |
| | White (n) | 28 |

(SD)—standard deviation;
n—number of subjects

### Adverse Events

No serious or severe adverse effects were reported. There were no meaningful relationships between increases in NMD670 dose and the incidence of participants with AEs. A total of 70 AEs were reported, of which 47 (67%) were at least possibly drug related. There were no relationships between increases in dose and the incidence of these individual AEs following administration of a single dose of NMD670, except for transient myotonia which was reported at the highest dose levels tested (1200 mg and 1600 mg). Most AEs were mild, except for one AE of myotonia (1600 mg), and tooth extraction (unrelated, 50 mg), which were moderate in intensity. There were no subject discontinuations.

### Pharmacokinetic Results

The pharmacokinetic results from the volunteers is given in table 12 below.

TABLE 12
Pharmacokinetic Results

| | | AUC_{0-infinity} | Cₘₐₓ | T_{1/2} |
|---|---|---|---|---|
| | | (Mean ± SD, | (Mean ± SD, | (mean ± SD, |
| Dose of NMD670 | | h · ng/mL) | ng/mL) | hr) |
| 50 | mg | — | 1900 ± 580 | — |
| 100 | mg | 18500 ± 1770 | 3980 ± 1190 | 5.07 ± 0.63 |
| 200 | mg | 36100 ± 5030 | 7780 ± 1130 | 4.6 ± 0.67 |
| 400 | mg | 87700 ± 21400 | 20100 ± 7340 | 4.68 ± 0.49 |
| 800 mg fasted | | 191000 ± 31500 | 36700 ± 15400 | 5.39 ± 0.82 |
| (male) | | | | |
| 800 mg fed | | 180000 ± 27900 | 37300 ± 7420 | 5.02 ± 1.04 |
| (male) | | | | |
| 800 mg | | 256000 ± 56200 | 44400 ± 7480 | 5.55 ± 0.55 |
| (female) | | | | |
| 1200 | mg | 358000 ± 176000 | 55000 ± 21700 | 6.11 ± 1.28 |
| 1600 | mg | 826000 | 123000 | 4.48 |

*AUC₀₋ₗₐₛₜ (Mean ± SD, h · ng/mL).
“—” indicates the information is not available.

### Example 6: Phase IIA Clinical Trial

A Phase 2, multicentre, randomised, double-blind, placebo-controlled, 2-way crossover proof-of-concept study aims to evaluate the clinical efficacy on muscle strength and function, safety, and tolerability of NMD670 administered daily for 3 weeks in ambulatory adults with Type 3 SMA.

### Overall Design Summary:

This is a Phase 2, multicentre, randomised, double-blind, placebo-controlled, 2-way crossover study.

The study will enrol 18 to 60-year-old male and female ambulatory participants diagnosed with Type 3 SMA with neuromuscular junction (NMJ) deficits. After screening, eligible participants will be randomised to either receive NMD670 (treatment period 1) followed by placebo (treatment period 2) or receive placebo (treatment period 1) followed by NMD670 (treatment period 2). Both the participant and Investigator will be blinded to treatment sequence assignment and will not know which treatment is given during each treatment period.

During the study, participants will continue receiving their usual SMA treatment per standard of care. Participants who receive SMA treatment that is not allowed during the study will not be eligible to participate in the study.

### Objectives and Endpoints

| Objectives | Endpoints |
|---|---|
| Primary Objective | |
| To assess changes in muscle strength | Change from baseline in 6MWT (total |
| and function after NMD670 400 mg bid | distance) after 21-day dosing |
| for 21 days, compared with placebo, in | |
| participants with SMA | |
| Secondary Objectives | |
| To assess changes in muscle strength | Change from baseline in individual |
| after NMD670 400 mg bid for 21 days, | muscle groups maximum strength |
| compared with placebo, in participants | (measured with a dynamometer) after |
| with SMA | 21-day dosing |
| To further assess changes in muscle | Change from baseline after 21-day |
| strength and function after NMD670 | dosing in: |
| 400 mg bid for 21 days, compared with | 6MWT (fatigue index) |
| placebo, in participants with SMA | revised Hammersmith scale score |
| | ESNHP |
| To assess changes in neuromuscular | Change from baseline in jitter and |
| junction transmission after NDM670 | blocking measured via sfEMG after 21- |
| 400 mg bid for 21 days, compared with | day dosing |
| placebo, in participants with SMA | |
| To assess the safety and tolerability of | AEs, physical examinations, clinical |
| NMD670, compared with placebo, over | laboratory parameters, vital signs, ECG, |
| 21-day dosing, in participants with SMA | C-SSRS |
| Exploratory Objectives | |
| To assess changes in patient-reported | Change from baseline after 21-day dosing |
| quality of life and fatigue severity after | in: |
| 21-day dosing of NMD670, compared | INQoL score |
| with placebo, in participants with SMA | FSS |

Abbreviations: 6MWT = 6-minute walk test; AE = adverse event; C SSRS = Columbia-Suicide Severity Rating Scale; ECG = electrocardiogram; ESNHPT = Endurance Shuttle Nine Hole Peg Test; FSS = fatigue severity scale; INQoL = Individualised Neuromuscular Quality of Life; sfEMG = single fibre electromyography; SMA = spinal muscular atrophy.

### Overall Design

The study duration will be approximately 8 to 13 weeks for each participant and include the following periods:
Screening period: up to 28 days
Baseline 1: 1 day
Treatment period 1: 21 (±1) days
Washout period: 6 (±1) days
Baseline 2: 1 day
Treatment period 2: 21 (±1) days
Follow-up period: 7 (±2) days

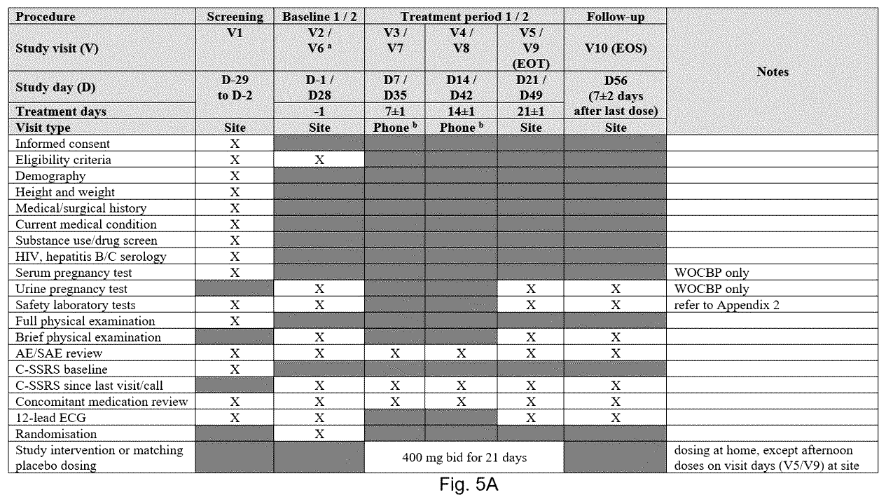

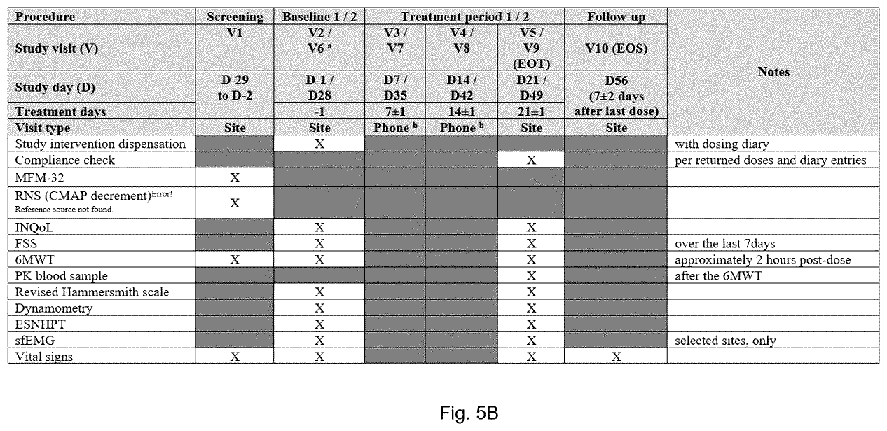

The study diagram is displayed FIG. 4 and the Schedule of Activities is provided in FIG. 5.

After screening, approximately 54 eligible participants will be randomised in a 1:1 ratio to either receive NMD670 (treatment period 1) followed by placebo (treatment period 2) or receive placebo (treatment period 1) followed by NMD670 (treatment period 2). Both the participant and Investigator will be blinded to treatment sequence assignment and will not know which treatment is given during each treatment period.

### Scientific Rationale for Study Design

The 6 minute walk test (6MWT) total distance was selected as the primary variable of the study to assess the effect of NMD670 after chronic dosing. The 6MWT can be safely performed in ambulatory patients with SMA. The 6MWT total distance is sensitive to fatigue related changes and the average distance walked each minute decreases between the first and the last minute of the test (Montes et al 2010). In addition to the total distance, the fatigue index will be included as secondary variable and calculated as percentage reduction in distance walked in 6th minute compared to 1st minute. Fatigability has been linked to NMJ transmission deficits in SMA and modulation of NMJ transmission has been proposed as a means to improve fatigability in SMA (Arnold et al 2021, Stam et al 2018b). The Endurance Shuttle Nine Hole Peg Test (ESNHPT) was selected as secondary assessment to determine the effect of NMD670 on upper extremity fatigability (Bartels et al 2019). Reliability and validity have been confirmed and the test is able to discriminate between SMA Types (Bartels et al 2020).

Muscle strength, as measured by dynamometry, was selected as the secondary variable to assess the effect of NMD670, as it has been observed that improvements in neuromuscular junction are associated with immediate increases in muscle strength (Pedersen et al 2016). Nonclinical studies have consistently shown that administration of CIC-1 inhibitors can improve muscle force production in different disease models characterised by NMJ dysfunction (Pedersen et al 2021).

The revised Hammersmith scale score was selected as secondary variable to assess the effect of NMD670 on functional abilities. The revised Hammersmith scale is a tool able to capture a broad range of abilities across the spectrum of SMA, from young children to adults, and at varying stages in the disease course. The revised Hammersmith scale has been validated in patients with SMA and has improved the ability to monitor changes at the 2 extremes of the scale compared to previous versions of the scale (Ramsey et al 2017).

At selected sites where single fibre electromyography (sfEMG) is conducted routinely, the PD effect on the neuromuscular junction will also be assessed in each treatment period by sfEMG, at baseline and after the last dose of study intervention. With the crossover design, participants will be their own control, reducing the impact of inter subject variability. The treatment sequence will be randomised to account to potential changes due to repeated assessments.

To avoid possible carry-over effect, a washout period of 6 (±1) days was included before the baseline of the second treatment period. With a half-life of approximately 4 hours, NMD670 should be eliminated 1 or 2 days after the last dose.

### Inclusion Criteria

Participants are eligible to be included in the study only if all of the following criteria apply:

### Age

1. Participant must be 18 to 60 years of age inclusive, at the time of signing the informed consent.

### Type of Participant and Disease Characteristics

2. Participants who are with a clinical diagnosis of Type 3 SMA.
3. Participants who are ambulatory, defined as being able to walk at least 50 metres without walking aids.
4. Participant with genetic confirmation of diagnosis (i.e., homozygous deletion of survival of motor neuron 1 gene [SMN1]).
5. Participant with 3 to 5 copies of survival of motor neuron 2 gene [SMN2].
6. Participants with a MFM-32 dimension 1 score <80% at screening.
7. Participants with ≥7% CMAP amplitude decrement at screening (recorded from the trapezius muscle during repeated nerve stimulation [RNS]).

### Weight

8. Participant has a body mass index (BMI) within the range 19-30 kg/m² (inclusive).

### Sex and Contraceptive/Barrier Requirements

9. Participant is male or female.
10. Contraceptive use by men and women must be consistent with local regulations regarding the methods of contraception for those participating in clinical studies.
   Male Participants:
   A male participant must agree to use a highly effective contraception during the intervention period and for at least 14 days after the last dose of study intervention (corresponding to time needed to eliminate study intervention) and refrain from donating sperm during this period.
   Female Participants:
   A female participant is eligible to participate if she is not pregnant, not breastfeeding, and at least one of the following conditions applies:
   Not a woman of childbearing potential (WOCBP).
   OR
   A WOCBP who agrees to follow the contraceptive guidance during the intervention period and for at least 14 days after the last dose of study intervention (corresponding to time needed to eliminate study intervention).

### Informed Consent

11. Participant is capable of giving signed informed consent which includes compliance with the requirements and restrictions listed in the informed consent form (ICF) and in this protocol.

### Exclusion Criteria

Participants are excluded from the study if any of the following criteria apply:

### Medical Conditions

1. Participants with prior surgery or fixed deformity (scoliosis, contractures) which would restrict ability to perform study-related tasks.
2. Participants with other significant disease that may interfere with the interpretation of study data (e.g., other neuromuscular or muscular diseases).
3. Participant with a clinical diagnosis of gout, or with serum uric acid >6.5 mg/dL at screening.
4. Participant with clinically significant ECG abnormalities at screening including PR interval ≥220 msec, irregular rhythms (other than sinus arrhythmia or occasional, rare supraventricular or rare ventricular ectopic beats) in the judgement of the Investigator, or T-wave configurations are not of sufficient quality for assessing QT interval duration.
5. Participant with any of the following abnormalities in QT interval corrected for heart rate using Fridericia's correction (QTcF) at screening:
   a. QTcF >450 msec for male participants without bundle branch block.
   b. QTcF >470 msec for female participants without bundle branch block.
   c. QTcF >480 msec in participants with bundle branch block.
6. Participant with any of the following:
   a. Abnormal liver function test defined as total bilirubin >1.5×ULN (participants with Gilbert's syndrome can be included with total bilirubin >1.5×ULN if direct bilirubin is ≤1.5×ULN and ≤35% of total bilirubin).
   b. Current or chronic history of liver disease including (but is not limited to) hepatitis virus infections, drug- or alcohol-related liver disease, non-alcoholic steatohepatitis, autoimmune hepatitis, hemochromatosis, Wilson's disease, α-1 antitrypsin deficiency, primary biliary cholangitis, primary sclerosing cholangitis, or any other liver disease considered clinically significant by the Investigator.
   c. Known hepatic or biliary abnormalities (with the exception of Gilbert's syndrome or asymptomatic gallstones).
7. Participant with clinically significant laboratory test abnormalities at screening.
8. Participant with breast cancer within the past 10 years or lymphoma, leukaemia, or any malignancy within the past 5 years. An exception of this 5-year requirement is basal cell or squamous epithelial carcinomas of the skin that have been resected with no evidence of metastatic disease for 5 years.
9. Participants with ongoing significant psychiatric disorder (e.g., uncontrolled depression, anxiety).
10. Participants with positive drug screen (cocaine, heroin, ketamine [unless medically prescribed], opiates) at screening.
11. Participants with positive human immunodeficiency virus (HIV) antibody test.
12. Participants with presence of hepatitis B surface antigen (HBsAg) [or hepatitis B core antibody (HBcAb)] at screening or within 3 months prior to first dose of investigational intervention.
13. Participants with positive hepatitis C antibody or ribonucleic acid (RNA) test result at screening or within 3 months prior to Day 1.
   NOTE: Participants with hepatitis C with positive hepatitis C antibody could be enrolled, if a negative hepatitis C RNA test is obtained.

### Prior/Concomitant Therapy

14. Participants who have received any prohibited medication within 30 days (or 5 half-lives of the medication, whichever is longer), or are likely to require treatment with prohibited medications during the study. Prohibited medications are listed in the Prior and Concomitant Therapy section and include drugs affecting neuromuscular transmission (such as anticholinergic drugs), drugs showing relevant effect on CIC-1 channel, drugs with potential drug-drug interactions with NMD670.

### Prior/Concurrent Clinical Study Experience

15. Participants received treatment with an investigational medical product (IMP) within 30 days (or 5 half-lives of the medication, whichever is longer) prior to Day 1.

### Diagnostic Assessments

16. Participants unable to perform or tolerate electromyography (EMG), according to medical history or at screening.

### Other Exclusion Criteria

17. Participants with history of poor compliance with relevant SMA therapy.

### Administration of Study Intervention

| Intervention | NMD670 | Placebo |
|---|---|---|
| Dosage Formulation | tablet | tablet |
| Unit Dose | 100-mg and 300-mg | matching tablets with |
| Strength(s) | tablets | no active treatment |
| Dosage Level(s) | 1 × 300-mg + 1 × 100-mg | 2 tablets bid |
| | tablets = 400 mg bid | |
| Route of | oral | oral |
| Administration | | |

Placebo or NMD670 tablets should be swallowed as whole tablets together with 240 mL of water.

One day before each treatment period, participants will attend a baseline visit at the study site and be dispensed with tablets to be taken at home. The next day, participants will start their treatment at home for 21 days at approximately the same time every day in the morning and 6 hours later in the afternoon (within 5 to 8 hours after the morning dose). Participants who miss a morning and/or an afternoon dose should not attempt to add or recover the missed dose at a later time or the next day. On the last day of each treatment period, participant will attend a visit to the study site. They will be asked to take the morning dose at home and the second dose will be administered at the site at approximately noon. If participants forget to take the morning dose at home before the visit, it will be administered at the site.

### Assignment to Study Intervention

All participants will be centrally randomised using an interactive response technology (IRT). Each participant will be assigned a unique number (randomisation number) that encodes the participant's assignment to 1 of the 2 treatment sequences of the study, according to the randomisation schedule generated using a validated computer program.

Study intervention will be administered/dispensed at the study visits as summarised in the Schedule of Activities (FIG. 5).

### Study Intervention Compliance

When participants self-administer study intervention(s) at home, they will be instructed to record the time of each morning and afternoon dosing in a diary every day and return unused tablets to the study site. Compliance with study intervention will be assessed by the Investigator when participants return to the site on the last day of the treatment period, by reviewing the diary entries and counting returned tablets. Compliance will be documented in the source documents and relevant form. Deviation(s) from the prescribed dosage regimen should be recorded.

When participants are dosed at the site, they will receive study intervention directly from the Investigator or designee, under medical supervision. The date and time of each dose administered in the study site will be recorded in the source documents. A record of the quantity of tablets dispensed to and administered by each participant must be maintained and reconciled with study intervention and compliance records. Intervention start and stop dates, including dates for intervention delays and/or dose reductions will also be recorded.

### Prior and Concomitant Therapy

Any medication or vaccine (including over-the-counter or prescription medicines, recreational drugs, vitamins, and/or herbal supplements) that the participant is receiving at the time of enrolment or receives during the study must be recorded along with:
reason for use
dates of administration including start and end dates
dosage information including dose and frequency

The medical monitor should be contacted if there are any questions regarding concomitant or prior therapy.

### Prior and Concomitant Medications Review

The Investigator or qualified designee will review prior medication use, and record prior medications taken by the participant within 30 days prior to screening as well as all relevant SMA therapies since diagnosis.

The Investigator or qualified designee will record medication, if any, taken by the participant during the study through the last visit. Concomitant medications will be recorded for 14 days after the last dose of study intervention (or longer if related to an SAE).

Participants using corticosteroids (for any reason) should maintain a stable dose for at least 3 months before the first dose of study intervention, and should be expected to require no dose changes until the end of the study.

SMA disease modifying drugs and/or symptomatic treatment modalities are allowed but doses should not be changed, adjusted or interrupted during the study:
Evrisdy (risdiplam) and Spinraza (nusinersen) should be maintained at a stable dose for at least 6 months prior to screening and until the end of the study.
If treated with Spinraza (nusinersen), the last dose prior to study entry should have been administered at least 1 month prior to screening and the participant should not receive any additional dose until the end of the study.
Zolgesma (onasemnogene abeparvovec) dose should have been administered at least 12 months prior to screening.

Other current and recent (within 1 month prior to the screening) treatments will be allowed, if judged by the Investigator to have no clinical relevance.

The following medications are prohibited during the study:
Drugs interfering with neuromuscular transmission such as anticholinergic drugs (e.g., atropine, benztropine, orphenadrine, scopolamine)
Muscle relaxants (e.g., carisoprodol, cyclobenzaprine, diazepam, metaxalone) NOTE: Short-acting benzodiazepines used for night-time treatment of insomnia are permitted (eg temazepam, triazolam, estazolam)
Drugs lowering serum uric acid:
Xanthine oxidase inhibitors (eg. allopurinol, febuxostat, topiroxostat)
Uricosuric agents (eg. benzbromarone, probenecid, arhalofenate)
Potent or moderately potent inhibitors of OATP1B1, BCRP, OAT1, OAT3, MATE2K, or OAT2 (e.g., probenecid, diclofenac, pyrimethamine)
Sensitive or moderately sensitive substrates of OAT3, BCRP, and URAT1 (e.g., topetecan, rosuvastatin, oseltamivir, olmesartan)

COVID-19 vaccines should be avoided from 1 week prior to screening to 1 week after the last dose.

### Discontinuation of Study Intervention

Participants may be discontinued from study intervention in the following situations:
Participant decision. The participant is at any time free to discontinue treatment, without prejudice to further treatment
An AE that, in the opinion of the Investigator and/or Sponsor, warrants discontinuation from further dosing
The participant experiences suicidal ideation and behaviour (SIB) that warrants discontinuation from further dosing after risk assessment
Safety reasons as judged by the Investigator and/or Sponsor where continued treatment may put the participant at undue risk

Participants will be discontinued from study intervention in the following situations:
The participant becomes pregnant during the course of the study
The participant has the following liver chemistry findings:
   AST or ALT >5×ULN or, if abnormal at baseline, >5×baseline value.
   AST or ALT >3×ULN and total bilirubin >2×ULN.
      For participants with known Gilbert's syndrome these criteria only apply if total bilirubin ≥2×ULN, and direct bilirubin >2×ULN and at least doubled from baseline value.
   AST or ALT >3×ULN and international normalised ratio (INR)>1.5.
   AST or ALT >3×ULN with the appearance of fatigue, nausea, vomiting, right upper quadrant pain or tenderness, fever, rash, and/or eosinophilia (>5%).

The participant has the following QTcF findings:
Participant without bundle branch block:
   QTcF >500 msec or change from baseline of QTcF >60 msec
Participant with bundle branch block:
   QTcF >500 msec if baseline value <450 msec
   QTcF ≥530 msec if baseline value ≥450 to 480 msec

If study intervention is permanently discontinued, the participant should, if at all possible, remain in the study to be evaluated for safety follow-up. See the Schedule of Activities (FIG. 5) for data to be collected at the time of follow-up and for any further evaluations that need to be completed.

### Administrative and Baseline Procedures

### Medical History

A medical history will be obtained by the Investigator or qualified designee. The medical history will collect all active conditions and any condition diagnosed that the Investigator considers to be clinically relevant. Details regarding the disease for which the participant has enrolled in this study will be recorded separately and not listed as medical history.

### Efficacy Assessments

The efficacy assessments planned at each visit are provided in the Schedule of Activities (FIG. 5).

Clinical assessments will be performed by a trained evaluator at the site (the Investigator or a physical therapist). For each test, each participant should be assessed by the same evaluator for all sessions. The use of assistive devices such as ankle foot orthoses may or may not be permitted during assessments, but should be the same for all sessions.

Before and after each clinical assessment, the assessors should ensure that participants are sufficiently rested, including at least 10 minutes of rest after each clinical assessment.

### Primary Efficacy Assessment: 6-Minute Walk Test (6MWT)

The 6MWT will be performed according to the American Thoracic Society (ATS) guidelines (ATS Committee on Proficiency Standards for Clinical Pulmonary Function Laboratories. ATS statement: guidelines for the six-minute walk test. Am J Respir Crit Care Med. 2002; 166(1):111-7. Erratum in: Am J Respir Crit Care Med. 2016; 193(10):1185, the contents of which are incorporated herein by reference). Participants will be instructed to walk as fast as possible along a 25-metre linear marked course for 6 minutes.

The distance walked during each minute will be recorded, so that the primary variable of total distance covered in 6 minutes can be determined, as well as other derived variables (e.g., fatigue index, change in speed index).

An increase in the total distance walked can be determined by comparing the change from baseline in the total distance walked after 21 days of NMD670 treatment with the change from baseline in the total distance walked after 21 days of placebo treatment.

### Other Clinician-rated Outcomes

### Dynamometry

Muscle strength will be assessed in muscle groups of upper and lower body extremities using dynamometry (Febrer A, Rodriguez N, Alias L, Tizzano E. Measurement of muscle strength with a handheld dynamometer in patients with chronic spinal muscular atrophy. J Rehabil Med. 2010 March; 42(3):228-31; Merlini L, Mazzone E S, Solari A, Morandi L. Reliability of hand-held dynamometry in spinal muscular atrophy. Muscle Nerve. 2002 July; 26(1):64-70; the contents of which are incorporated herein by reference). Strength of individual muscle groups will be included as secondary endpoint. Dynamometry will be performed as scheduled in the Schedule of Activities (FIG. 5).

Participants will be instructed to contract as forcefully as possible, using a CITEC Hand-Held Dynamometer (HDD) Model 3002/60. 3 attempts will be given for every muscle group, with a minimum of 30 seconds between each measurement, and the highest value recorded as peak force.

The assessment of individual muscle groups should, if possible, be assessed in the order outlined as follows: hand grip, shoulder abductor, elbow flexors, elbow extensors, knee flexors.

### Revised Hammersmith Scale

The revised Hammersmith scale is a functional clinician-rated outcome measure that was developed to assess physical abilities of patients with SMA, from those with weaker muscle strength Type 2 SMA to those with stronger muscle strength in ambulatory Type 3 SMA (Ramsey D, Scoto M, Mayhew A, Main M, Mazzone E S, Montes J, et al. Revised Hammersmith Scale for spinal muscular atrophy: A SMA specific clinical outcome assessment tool. PLoS One. 2017; 12(2):e0172346, the contents of which are incorporated herein by reference).

The scale consists of 36 items and 2 timed tests:
33 items are graded on an ordinal scale of 0, 1, 2 where 0 denotes the least level of ability/function progressing to the highest level of ability to achieve a score of 2.
3 items are scored 0, 1 where 0 is unable and 1 is able to achieve.
Timed tests are included for 2 items:
   Item 19: time to run 10 metres (as quickly and as safely as possible)
   Item 25: time to rise from floor (using as little support as possible and as fast as possible)

The maximum achievable total score is 69.

### Endurance Shuttle Nine Hole Peg Test (ESNHPT)

The ESNHPT will be performed according to standardised procedures (Bartels B, Habets L E, Stam M, Wadman R I, Wijngaarde C A, Schoenmakers MAGC et al. Assessment of fatigability in patients with spinal muscular atrophy: development and content validity of a set of endurance tests. BMC Neurol. 2019; 19(1):21, the contents of which are incorporated herein by reference).

Participants will be instructed to repeatedly place and return 9 pegs in 9 holes at 75% of their maximum speed (predetermined before the test for each participant). Each round will be paced by an auditory signal and the test will end when the participant is unable to keep up with the pre-set pace on 2 consecutive rounds, with a maximum duration of 20 minutes.

Two outcomes will be recorded:
Dropout (yes/no): defined as the inability to endure the maximum test duration of 20 minutes.
Time to limitation: time when the participant is no longer able to keep up with pre-set pace and the test stops.
   Motor Function Measure 32-item (MFM-32)

The MFM-32, a test that is only performed during screening, consists of 32 task items in 3 dimensions that provide a detailed profile of the physical impairment (Vuillerot C, Payan C, Iwaz J, Ecochard R, Berard C; MFM Spinal Muscular Atrophy Study Group. Responsiveness of the motor function measure in patients with spinal muscular atrophy. Arch Phys Med Rehabil. 2013; 94(8):1555-61, the contents of which are incorporated herein by reference):
D1: standing and transfers
D2: axial and proximal motor function
D3: distal motor function

The scoring of each task uses a 4-point Likert scale based on the participant's maximal abilities without assistance:
0: cannot initiate the task or maintain the starting position
1: performs the task partially
2: performs the task incompletely or imperfectly (with compensatory/uncontrolled movements or slowness)
3: performs the task fully and “normally.”

The 32 scores are summed to yield a total score expressed as the percentage of the maximum possible score (the one obtained with no physical impairment); the lower the total score, the more severe the impairment.

### Patient-Reported Outcomes

Participants will be asked to complete 2 questionnaires, the fatigue severity scale (FSS) and the Individualised Neuromuscular Quality of Life (INQoL), to assess how their fatigue interferes with their activities and how their SMA impacts their quality of life.

Participants will be given the questionnaires as paper forms to complete. They will be allowed to use a proxy if they have physical limitations preventing them from filling out the questionnaires.

### Fatigue Severity Scale (FSS)

The FSS is a questionnaire initially validated in patients with multiple sclerosis and systemic lupus erythematosus to quantify the degree of subjective fatigue, but is also used in patients with SMA (Kizina K, Stolte B, Totzeck A, Bolz S, Schlag M, Ose C, et al. Fatigue in adults with spinal muscular atrophy under treatment with nusinersen. Sci Rep. 2020; 10(1):11069, the contents of which are incorporated herein by reference).

The FSS questionnaire consists of 9 questions related to how fatigue interferes with certain activities and rates its severity according to a self-report scale. Each item is scored on a 7-point scale from 1 (strongly disagree) to 7 (strongly agree). The total score ranges from a minimum of 9 to a maximum of 63.

### Individualised Neuromuscular Quality of Life (INQoL)

The INQoL questionnaire was developed in patients affected by different muscle diseases (muscular dystrophies, inflammatory and congenital myopathies) to specifically detect functional limitations relevant to patients with neuromuscular disorders (Vincent K A, Carr A J, Walburn J, Scott D L, Rose M R. Construction and validation of a quality of life questionnaire for neuromuscular disease (INQoL). Neurology. 2007; 68(13):1051-7; Sadjadi R, Vincent K A, Carr A J, Walburn J, Brooks V L, Pandya S, et al. Validation of the individualised neuromuscular quality of life for the USA with comparison of the impact of muscle disease on those living in USA versus UK. Health Qual Life Outcomes. 2011; 9:114, the contents of which are incorporated herein by reference).

The INQoL questionnaire consists of 45 self-administered questions within 10 sections related to the physical health domain, and the areas of life and psycho-social aspects:
The physical health domain pertains to the impact of common neuromuscular symptoms (i.e., weakness, locking, pain and fatigue domains) on quality of life, Participants respond to each section first with a “yes/no” answer, determining if that specific aspect of the disease impacts their daily lives. If yes, it is then asked to determine both the degree of the impact of that topic on their life, and the importance that they attribute to it, by 7-point Likert scales.
Domains of activities, dependence, body image, relationships and emotions evaluate the impact of the disease on psychological and social functioning. Participants are directly asked to rate the impact of their condition on specific aspects in each domain, by 7-point Likert scales.

### Safety Assessments

The safety assessments planned at each visit are provided in the Schedule of Activities (FIG. 5).

### Height and Weight

Height and weight will be measured and recorded.

### Physical Examinations

A complete physical examination will include, at a minimum, assessments of the neurological, cardiovascular, respiratory, gastrointestinal, musculoskeletal and skin systems.
A brief physical examination will include, at a minimum, assessments of the neurological and musculoskeletal systems.

Investigators should pay special attention to clinical signs related to previous serious illnesses.

### Vital Signs

Vital signs will be measured in a supine position after 5 minutes rest and will include tympanic temperature, systolic and diastolic blood pressure, and pulse and respiratory rate.

Three readings of blood pressure and pulse will be taken at least 1 minute apart. For orthostatic vital signs measurements, participants will be supine for at least 5 minutes before obtaining a single measurement of blood pressure and pulse. Participants will then stand for at least 2 minutes before obtaining a single measurement of blood pressure and pulse.

### Electrocardiograms

Triplicate 12-lead ECG(s) will be obtained as outlined in the Schedule of Activities (FIG. 5) using an ECG machine that automatically calculates the heart rate and measures PR, QRS, QT, RR, and QTcF intervals.

For triplicate ECG recordings, 3 individual ECG tracings should be obtained as closely as possible in succession, but no more than 2 minutes apart.

For participants showing an increase of QTcF by ≥60 msec (regardless baseline value) or abnormalities in QTcF (as defined in exclusion criterion 5), persisting for ≥5 minutes, further evaluation is recommended, including repeated ECGs. A cardiologist may be consulted. Refer to Discontinuation of Study Intervention section for QTcF withdrawal criteria.

The central ECG review vendor, as the responsible company for the centralised ECG assessments, will provide the study sites with standardised ECG equipment and supplies, specific training and written instructions. Following an acquisition of a quality ECG tracing, the Investigator or designee will electronically transfer the data to the central ECG review vendor.

Investigators will assess the eligibility of the participant according to the ECG report of Visit 1. Any abnormal finding in the ECG tracing will be evaluated by the Investigator or by a hospital cardiologist during the visit and will be specifically documented and registered in the CRF. Throughout the study, clinically relevant new findings or worsening of a pre-existing finding in the ECGs (parameters or abnormal findings in the tracing) must be considered an AE and must be recorded in the AE CRF form.

### Adverse Events (AEs), Serious Adverse Events (SAEs), and Other Safety Reporting

The Investigator and any qualified designees are responsible for detecting, documenting, and reporting events that meet the definition of an AE or SAE and remain responsible for following up all AEs (see Discontinuation of Study Intervention section). This includes events reported by the participant (or, when appropriate, by a caregiver, surrogate, or the participant's legally authorised representative).

### Time Period and Frequency for Collecting AE and SAE Information

All AEs and SAEs will be collected from the signing of ICF until the follow-up visit, as specified in the Schedule of Activities (FIG. 5).

All SAEs will be recorded and reported to the Sponsor or designee within 24 hours of awareness. The Investigator will submit any updated SAE data to the Sponsor within 24 hours of it being available.

Investigators are not obliged to actively seek information on AEs or SAEs after the conclusion of study participation. However, if the Investigator learns of any SAE, including a death, at any time after a participant has been discharged from the study, and the Investigator considers the event to be reasonably related to the study intervention or study participation, the Investigator must promptly notify the Sponsor.

### Pharmacokinetics

A single whole blood sample of approximately 3 mL will be collected for measurement of plasma concentrations of NMD670 and its metabolite as specified in the Schedule of Activities (FIG. 5). The timing of sampling may be altered during the course of the study based on newly available data (e.g., to obtain data closer to the time of peak plasma concentrations) to ensure appropriate monitoring. Instructions for the collection and handling of biological samples will be provided by the Sponsor. The actual date and time (24 hour clock time) of each sample collection will be recorded. Genetic analyses will not be performed on these blood samples.

In brief, blood will be processed and plasma analysed using validated assays.

Intervention concentration information that would unblind the study will not be reported to investigative sites or blinded personnel until the study has been unblinded.

### Pharmacodynamics

Single Fibre Electromyography (sfEMG)

Single fibre EMG will be performed in the tibialis anterior muscle and possibly in additional muscle groups (e.g., in the upper limb), as scheduled in the Schedule of Activities (FIG. 5).

Only a subset of selected sites where sfEMG is conducted routinely will perform this procedure.

The sfEMG electrode is a needle electrode with a small recording surface that allows for the recording of 1 or more single fibre action potentials from a given motor unit. Time-locked action potentials from at least 2 muscle fibres triggered by voluntary contraction of the tested muscle are recorded. Neuromuscular jitter represents the variation in time intervals between pairs of action potentials. Blocking represents the percentage of pairs in which the action potential is not observed for 1 of the fibres. Recordings will be performed using standardised equipment, settings, operating procedures and filters.

### Repeated Nerve Stimulation (RNS)

The RNS test, a test that is only performed during screening, will be performed at screening, by recording CMAP amplitude from the trapezius muscle during a train of supramaximal stimuli.

Recordings will be performed using standardised equipment, settings, and operating procedures.

The CMAP change could be negative (decrement) or positive (increment) and will be calculated as a percentage as follows:

$$CMAP change = \frac{{4}^{th} CMAP amplitude - {1}^{st} CMAP amplitude}{{1}^{st} CMAP amplitude} \times 100$$

### REFERENCES

Arnold et al 2015. Arnold W D, Porensky P N, McGovern V L, Iyer C C, Duque S, Li X, Meyer K, Schmelzer L, Kaspar B K, Kolb S J, Kissel J T, Burghes A H. Electrophysiological Biomarkers in Spinal Muscular Atrophy: Preclinical Proof of Concept. Ann Clin Transl Neurol. 2014 Jan. 1; 1(1):34-44. The entire content of the foregoing document is incorporated by reference herein.
Arnold et al 2021. Arnold W D, Severyn S, Zhao S, Kline D, Linsenmayer M, Kelly K, et al. Persistent neuromuscular junction transmission defects in adults with spinal muscular atrophy treated with nusinersen. BMJ Neurol Open. 2021; 3(2):e000164. The entire content of the foregoing document is incorporated by reference herein.
ATS 2002. ATS Committee on Proficiency Standards for Clinical Pulmonary Function Laboratories. ATS statement: guidelines for the six-minute walk test. Am J Respir Crit Care Med. 2002; 166(1):111-7. Erratum in: Am J Respir Crit Care Med. 2016; 193(10):1185. The entire content of the foregoing document is incorporated by reference herein.
Bartels et al 2019. Bartels B, Habets L E, Stam M, Wadman R I, Wijngaarde C A, Schoenmakers MAGC et al. Assessment of fatigability in patients with spinal muscular atrophy: development and content validity of a set of endurance tests. BMC Neurol. 2019; 19(1):21. The entire content of the foregoing document is incorporated by reference herein.
Bartels et al 2020. Bartels B, de Groot J F, Habets L E, Wijngaarde C A, Vink W, Stam M, et al. Fatigability in spinal muscular atrophy: validity and reliability of endurance shuttle tests. Orphanet J Rare Dis. 2020; 15(1):75. The entire content of the foregoing document is incorporated by reference herein.
De Sanctis et al 2016. De Sanctis R, Coratti G, Pasternak A, Montes J, Pane M, Mazzone E S, Young S D, Salazar R, Quigley J, Pera M C, Antonaci L, Lapenta L, Glanzman A M, Tiziano D, Muntoni F, Darras B T, De Vivo D C, Finkel R, Mercuri E. Developmental milestones in type I spinal muscular atrophy. Neuromuscul Disord. 2016 November; 26(11):754-759. The entire content of the foregoing document is incorporated by reference herein.
De Wel et al 2021. De Wel B, Goosens V, Sobota A, Van Camp E, Geukens E, Van Kerschaver G, Jagut M, Claes K, Claeys K G. Nusinersen treatment significantly improves hand grip strength, hand motor function and MRC sum scores in adult patients with spinal muscular atrophy types 3 and 4. J Neurol. 2021 March; 268(3):923-935. The entire content of the foregoing document is incorporated by reference herein.
Febrer et al 2010. Febrer A, Rodriguez N, Alias L, Tizzano E. Measurement of muscle strength with a handheld dynamometer in patients with chronic spinal muscular atrophy. J Rehabil Med. 2010 March; 42(3):228-31. The entire content of the foregoing document is incorporated by reference herein.
Foust et al 2010. Foust K D, Wang X, McGovern V L, et al. Rescue of the spinal muscular atrophy phenotype in a mouse model by early postnatal delivery of SMN. Nat Biotechnol 2010; 28:271-4. The entire content of the foregoing document is incorporated by reference herein.
Glanzman et al 2010. Glanzman A M, Mazzone E, Main M, Pelliccioni M, Wood J, Swoboda K J, Scott C, Pane M, Messina S, Bertini E, Mercuri E, Finkel R S. The Children's Hospital of Philadelphia Infant Test of Neuromuscular Disorders (CHOP INTEND): test development and reliability. Neuromuscul Disord. 2010 March; 20(3):155-61. The entire content of the foregoing document is incorporated by reference herein.
Kizina et al 2020. Kizina K, Stolte B, Totzeck A, Bolz S, Schlag M, Ose C, et al. Fatigue in adults with spinal muscular atrophy under treatment with nusinersen. Sci Rep. 2020; 10(1):11069. The entire content of the foregoing document is incorporated by reference herein.
Kong et al 2009. Kong L, Wang X, Choe D W, Polley M, Burnett B, Bosch-Marcé M, Griffin J W, Rich M M, Sumner C J. Impaired synaptic vesicle release and immaturity of neuromuscular junctions in spinal muscular atrophy mice. J Neurosci 2009; 29:842-51. The entire content of the foregoing document is incorporated by reference herein.
Maggi et al 2020. Maggi L, Bello L, Bonanno S, Govoni A, Caponnetto C, Passamano L, et al. Nusinersen safety and effects on motor function in adult spinal muscular atrophy type 2 and 3. J Neurol Neurosurg Psychiatry. 2020; 91(11):1166-74. The entire content of the foregoing document is incorporated by reference herein.
Main et al, 2003. Main M, Kairon H, Mercuri E, Muntoni F. The Hammersmith functional motor scale for children with spinal muscular atrophy: a scale to test ability and monitor progress in children with limited ambulation. Eur J Paediatr Neurol. 2003; 7(4):155-9. The entire content of the foregoing document is incorporated by reference herein.
Mazzone et al 20176. B Mazzone E S, Mayhew A, Montes J, Ramsey D, Fanelli L, Young S D, Salazar R, De Sanctis R, Pasternak A, Glanzman A, Coratti G, Civitello M, Forcina N, Gee R, Duong T, Pane M, Scoto M, Pera M C, Messina S, Tennekoon G, Day J W, Darras B T, De Vivo D C, Finkel R, Muntoni F, Mercuri E. Revised upper limb module for spinal muscular atrophy: Development of a new module. Muscle Nerve. 2017 June; 55(6):869-874. The entire content of the foregoing document is incorporated by reference herein.
Mercuri et al 2018. Mercuri E, Finkel R S, Muntoni F, Wirth B, Montes J, Main M, Mazzone E S, Vitale M, Snyder B, Quijano-Roy S, Bertini E, Davis R H, Meyer O H, Simonds A K, Schroth M K, Graham R J, Kirschner J, Iannaccone S T, Crawford T O, Woods S, Qian Y, Sejersen T, et al. Diagnosis and management of spinal muscular atrophy: part 1: recommendations for diagnosis, rehabilitation, orthopedic and nutritional care. Neuromuscul Disord. 2018; 28:103-115. The entire content of the foregoing document is incorporated by reference herein.
Merlini et al 2002. Merlini L, Mazzone E S, Solari A, Morandi L. Reliability of hand-held dynamometry in spinal muscular atrophy. Muscle Nerve. 2002 July; 26(1):64-70. The entire content of the foregoing document is incorporated by reference herein.
Montes et al 2010. Montes J, McDermott M P, Martens W B, Dunaway S, Glanzman A M, Riley S, et al. Six-Minute Walk Test demonstrates motor fatigue in spinal muscular atrophy. Neurology. 2010; 74(10):833-8. The entire content of the foregoing document is incorporated by reference herein.
O'Hagen et al 2007. O'Hagen J M, Glanzman A M, McDermott M P, Ryan P A, Flickinger J, Quigley J, Riley S, Sanborn E, Irvine C, Martens W B, Annis C, Tawil R, Oskoui M, Darras B T, Finkel R S, De Vivo D C. An expanded version of the Hammersmith Functional Motor Scale for SMA II and III patients. Neuromuscul Disord. 2007 October; 17(9-10):693-7. The entire content of the foregoing document is incorporated by reference herein.
Pedersen et al 2016. Pedersen T H, Riisager A, de Paoli F V, Chen T Y, Nielsen O B. Role of physiological CIC-1 Cl− ion channel regulation for the excitability and function of working skeletal muscle. J Gen Physiol. 2016; 147(4):291-308. The entire content of the foregoing document is incorporated by reference herein.
Pedersen et al 2021. Pedersen T H, Macdonald W A, Broch-Lips M, Halldorsdottir O, Baekgaard Nielsen O. Chloride channel inhibition improves neuromuscular function under conditions mimicking neuromuscular disorders. Acta Physiol (Oxf). 2021; 233(2):e13690. The entire content of the foregoing document is incorporated by reference herein.
Pera et al 2017. Pera M C, Luigetti M, Pane M, et al. 6MWT can identify type 3 SMA patients with neuromuscular junction dysfunction. Neuromuscul Disord 2017; 27:879-82. The entire content of the foregoing document is incorporated by reference herein.
Ramsey et al 2017. Ramsey D, Scoto M, Mayhew A, Main M, Mazzone E S, Montes J, et al. Revised Hammersmith Scale for spinal muscular atrophy: A SMA specific clinical outcome assessment tool. PLoS One. 2017; 12(2):e0172346. The entire content of the foregoing document is incorporated by reference herein.
Rodday et al 2017. Rodday A M, Graham R J, Weidner R A, Rothrock N E, Dewalt D A, Parsons S K. Leveraging pediatric PROMIS item banks to assess physical functioning in children at risk for severe functional loss. J Patient Rep Outcomes. 2017; 1(1):10. The entire content of the foregoing document is incorporated by reference herein.
Sadjadi et al 2011. Sadjadi R, Vincent K A, Carr A J, Walburn J, Brooks V L, Pandya S, et al. Validation of the individualised neuromuscular quality of life for the USA with comparison of the impact of muscle disease on those living in USA versus UK. Health Qual Life Outcomes. 2011; 9:114. The entire content of the foregoing document is incorporated by reference herein.
Sanders et at 2019. Sanders D B, Arimura K, Cui L, Ertaş M, Farrugia M E, Gilchrist J, Kouyoumdjian J A, Padua L, Pitt M, Stålberg E. Guidelines for single fiber EMG. Clin Neurophysiol. 2019 August; 130(8):1417-1439. The entire content of the foregoing document is incorporated by reference herein.
Stam et al 2018a. Stam M, Wadman R I, Bartels B, Leeuw M, Westeneng H J, Wijngaarde C A, et al. A continuous repetitive task to detect fatigability in spinal muscular atrophy. Orphanet J Rare Dis. 2018; 13(1):160. The entire content of the foregoing document is incorporated by reference herein.
Stam et al 2018b. Stam M, Wadman R I, Wijngaarde C A, Bartels B, Asselman F L, Otto L A M, et al. Protocol for a phase II, monocentre, double-blind, placebo-controlled, cross-over trial to assess efficacy of pyridostigmine in patients with spinal muscular atrophy types 2-4 (SPACE trial). BMJ Open. 2018; 8(7):e019932. The entire content of the foregoing document is incorporated by reference herein.
Vincent et al 2007. Vincent K A, Carr A J, Walburn J, Scott D L, Rose M R. Construction and validation of a quality of life questionnaire for neuromuscular disease (INQoL). Neurology. 2007; 68(13):1051-7. The entire content of the foregoing document is incorporated by reference herein.
Vuillerot et al 2013. Vuillerot C, Payan C, Iwaz J, Ecochard R, Berard C; MFM Spinal Muscular Atrophy Study Group. Responsiveness of the motor function measure in patients with spinal muscular atrophy. Arch Phys Med Rehabil. 2013; 94(8):1555-61. The entire content of the foregoing document is incorporated by reference herein.
Wadman et al 2012. Wadman R I, Vrancken AFJE, van den Berg L H, et al. Dysfunction of the neuromuscular junction in spinal muscular atrophy types 2 and 3. Neurology 2012; 79:2050-5. The entire content of the foregoing document is incorporated by reference herein.
Wang et al 2007. Wang C H, Finkel R S, Bertini E S, Schroth M, Simonds A, Wong B, Aloysius A, Morrison L, Main M, Crawford T O, Trela A., Participants of the International Conference on SMA Standard of Care. Consensus statement for standard of care in spinal muscular atrophy. J Child Neurol. 2007; 22:1027-49. The entire content of the foregoing document is incorporated by reference herein.
WHO 2006. WHO Multicentre Growth Reference Study Group. WHO Motor Development Study: windows of achievement for six gross motor development milestones. Acta Paediatr Suppl. 2006 April; 450:86-95. The entire content of the foregoing document is incorporated by reference herein.

1. A method of treating spinal muscular atrophy in a subject comprising administering a therapeutic dose of a composition containing (2S)-2-[4-bromo-2-(1,2-oxazol-3-yl)phenoxy]propanoic acid, a pharmaceutically acceptable salt thereof, a hydrate thereof, a polymorph thereof, a tautomer thereof, or solvate thereof, wherein the therapeutic dose ranges from 100 to 1500 mg.

2. The method according to claim 1, wherein the therapeutic dose ranges from 200 to 600 mg.

3. The method according to claim 1, wherein the therapeutic dose is administered one, two or three times daily.

4. The method according to claim 1, wherein the therapeutic dose is 400 mg and the therapeutic dose is administered two times daily.

5. The method according to claim 1, wherein the composition is administered orally.

6. The method according to claim 1, wherein the composition is a solid dosage form.

7. The method according to claim 6, wherein the composition comprises silicified microcrystalline cellulose.

8. The method according to claim 1, wherein the subject has a deletion or mutation in each survival motor neuron 1 (SMN1) allele.

9. The method according to claim 1, wherein the subject is suffering from SMA Type 0, SMA Type 1, SMA Type 2, SMA Type 3 or SMA Type 4.

10. The method according to claim 1, wherein the composition is to administered orally using a solid dosage form and provides a plasma concentration-time profile of (2S)-2-[4-bromo-2-(1,2-oxazol-3-yl)phenoxy]propanoic acid, the pharmaceutically acceptable salt, the hydrate thereof, the polymorph thereof, the tautomer thereof, or the solvate thereof where the mean Cₘₐₓ is 12,760 to 27,440 ng/mL after administration with a single dose of 400 mg of (2S)-2-[4-bromo-2-(1,2-oxazol-3-yl)phenoxy]propanoic acid, the pharmaceutically acceptable salt, the hydrate thereof, the polymorph thereof, the tautomer thereof, or the solvate thereof.

11. The method according to claim 1, wherein the subject experiences an increase in a total distance walked when determined using a 6-minute walk test after treatment with the composition.

12. A kit-of-parts comprising:
(2S)-2-[4-bromo-2-(1,2-oxazol-3-yl)phenoxy]propanoic acid, a pharmaceutically acceptable salt thereof, a hydrate thereof, a polymorph thereof, a tautomer thereof, or a solvate thereof, and
a compound that increases an amount of functional SMN protein produced by SMN2.

13. The kit-of-parts according to claim 12, wherein the kit-of-parts comprises 100 to 1500 mg of (2S)-2-[4-bromo-2-(1,2-oxazol-3-yl)phenoxy]propanoic acid, the pharmaceutically acceptable salt thereof, the hydrate thereof, the polymorph thereof, the tautomer thereof, or the solvate thereof.

14. The kit-of-parts according to claim 12, wherein the compound is selected from the group consisting of risdiplam, nusinersen, branaplam and BIIB115.

15. (canceled)

16. A composition comprising:
a therapeutic dose of (2S)-2-[4-bromo-2-(1,2-oxazol-3-yl)phenoxy]propanoic acid, a pharmaceutically acceptable salt thereof, a hydrate thereof, a polymorph thereof, a tautomer thereof, or solvate thereof, wherein the therapeutic dose ranges from 100 to 1500 mg.

17. The composition of claim 16, wherein the therapeutic dose ranges from 200 to 600 mg.

18. The composition of claim 16, wherein the therapeutic dose is 400 mg.

19. The composition of claim 16, wherein the composition is a solid dosage form and comprises silicified microcrystalline cellulose.
